(19) United States
(12) Patent Application Publication
(10) Pub. No.: US 2024/0383961 A1
(43) Pub. Date: Nov. 21, 2024

(54) CONJUGATED ANTIBODIES FOR TREATING DISEASES

(71) Applicants: Biohaven Therapeutics Ltd., New Haven, CT (US); AMDA Biologics, Inc., Boca Raton, FL (US)

(72) Inventors: Brian DOWD, North Haven, CT (US); James MOND, Boca Raton, FL (US); Adam GROSSMAN, Boca Raton, FL (US)

(21) Appl. No.: 18/692,392

(22) PCT Filed: Sep. 15, 2022
(86) PCT No.: PCT/US2022/043566

§ 371 Date: Mar. 15, 2024

Related U.S. Application Data
(60) Provisional application No. 63/245,180, filed on Sep. 16, 2021.

(51) Int. Cl. C07K 14/735; A61K 38/00; C07K 14/705

(52) U.S. Cl. CPC C07K 14/70535; C07K 14/7056; A61K 38/00; C07K 2319/30

(57) ABSTRACT

An agent including a human plasma immunoglobulin moiety, an immune cell surface receptor binding moiety capable of modulating the immune cell surface receptor, and optionally, a linker moiety linking the human plasma immunoglobulin moiety and the immune cell surface receptor binding moiety.

### CROSS-REFERENCE TO RELATED APPLICATIONS

This application claims priority to U.S. Provisional Application No. 63/245,180 filed Sep. 16, 2021, and all the benefits accruing therefrom under 35 U.S.C. § 119, the contents of which is incorporated herein in its entirety by reference.

### FIELD

The present disclosure relates to novel conjugates comprising human plasma derived immune globulin (IG) and one or more modulators of cell surface Fc receptors, compositions comprising the conjugates, methods of manufacturing the compositions, and methods of utilizing the compositions (e.g., for prophylactic administration and/or therapeutic treatment).

### BACKGROUND

Conjugated antibodies are useful for various purposes, for example, diagnostic reagents and therapeutics. There is a need for the development of enhanced IG products for use in various inflammatory, autoimmune, oncology and viral disorders.

### SUMMARY

The present disclosure is directed to immunoglobulin modified by conjugation to one or more immune cell surface receptor binding moieties (e.g., modulators that enhance or dampen Fc receptor mediated immune responses), compositions comprising the conjugates, methods of manufacturing the compositions, and methods of utilizing the compositions (e.g., for prophylactic administration and/or therapeutic treatment) for inflammatory, autoimmune, oncologic, pathogenic (e.g., viral pathogenic), and/or other disorders or conditions.

In an embodiment, provided is an agent including:

A human plasma immunoglobulin moiety,
an immune cell surface receptor binding moiety capable of modulating the immune cell surface receptor, and
optionally a linker moiety linking the human plasma immunoglobulin moiety and the immune cell surface receptor binding moiety.

In another embodiment, provided is a method of treating an acute or chronic inflammatory, oncological, or acute or chronic autoimmune disorder in a patient in need thereof, including administering to the patient a pharmaceutically effective amount of the agent.

In still another embodiment, provided is an agent including:
an hyperimmune globulin moiety,
an immune cell surface receptor binding moiety capable of modulating the immune cell surface receptor, and
optionally a linker moiety linking the hyperimmune globulin moiety and the immune cell surface receptor binding moiety.

In yet another embodiment, one or more agents disclosed herein are utilized for administration to a subject in need thereof (e.g., for prophylactic administration and/or therapeutic treatment). For example, in an embodiment, one or more agents disclosed herein are utilized for passive immunization (e.g., immune-prophylaxis). In a further embodiment, one or more disclosed agents (e.g., a pharmaceutically effective amount of one or more disclosed agents) are administered to a subject to prophylactically treat infection associated with a microbial pathogen. In another embodiment, one or more disclosed agents (e.g., a pharmaceutically effective amount of one or more disclosed agents) are administered to a subject to therapeutically treat infection associated with a microbial pathogen.

### BRIEF DESCRIPTION OF THE DRAWINGS

These and/or other aspects will become apparent and more readily appreciated from the following description of the embodiments, taken in conjunction with the accompanying drawings in which:

FIG. 1 is a graph of intensity of various glycans present in glycoproteins;

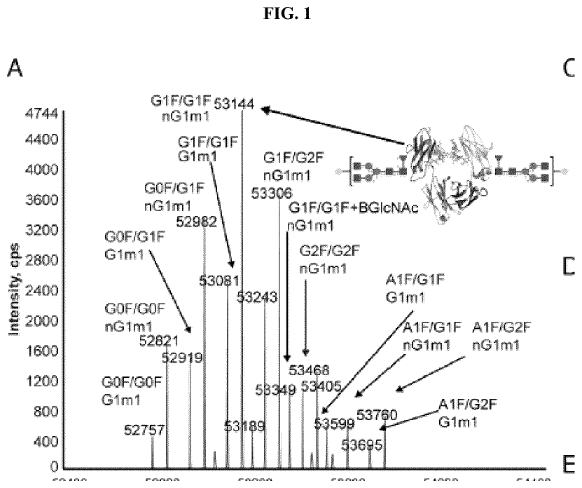

FIG. 2 is a diagram depicting a type II Fc cell surface receptor (C-type lectin dendritic cell-specific intercellular adhesion molecule 3-grabbing nonintegrin—DC-SIGN) that serves as a docking site for pathogens on the surface of dendritic cells and macrophages, and an example of how tetramerization of DC-SIGN and further clustering provides high binding avidity and can be used to influence/alter pathogen binding, as shown with HIV-1 binding to dendritic cells, with tetramerization increasing avidity to PAMPs like gp120 on the HIV-1 surface while DC-SIGN clusters act as functional docking sites for HIV-1;

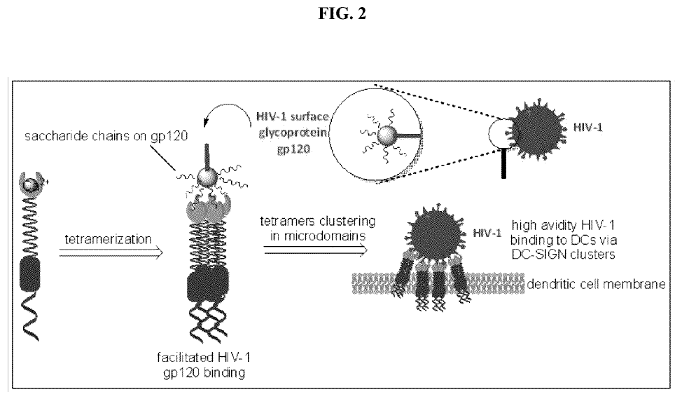

FIG. 3 is a diagram showing a human plasma immunoglobulin conjugate according to an embodiment of the present disclosure;

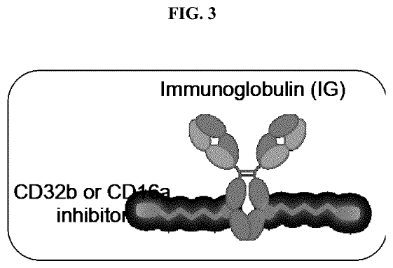

FIG. 4 is a diagram showing a hyperimmune globulin conjugate according to an embodiment of the present disclosure; and

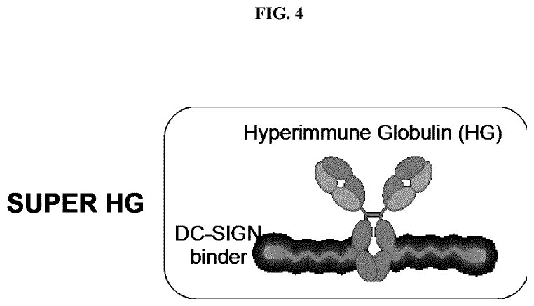

FIG. 5 illustrates IL-15 signaling pathways.

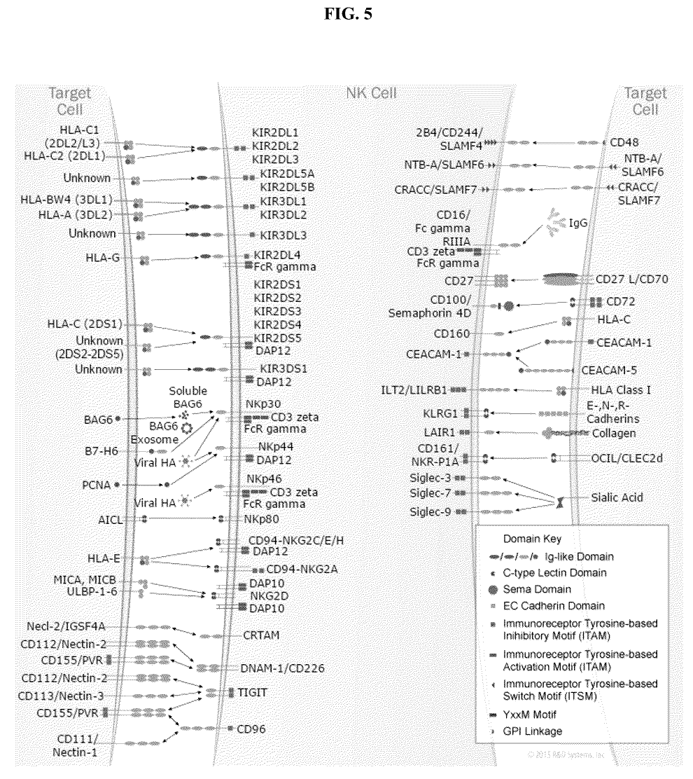

### DETAILED DESCRIPTION

The following detailed description is provided to aid those skilled in the art in practicing the present disclosure. Exemplary embodiments will hereinafter be described in detail. However, these embodiments are only exemplary, and the present disclosure is not limited thereto but rather is defined by the scope of the appended claims. Those of ordinary skill in the art may make modifications and variations in the embodiments described herein without departing from the spirit or scope of the present disclosure.

Accordingly, the embodiments are merely described below, by referring to structures and schemes, to explain aspects of the present description. As used herein, the term “and/or” includes any and all combinations of one or more of the associated listed items. The term “or” means “and/or.” Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

It is understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all technical and scientific terms used herein have the same meaning as commonly understood by one of ordinary skill in the art to which this disclosure belongs. The terminology used in the description is for describing particular embodiments only and is not intended to be limiting. It will be further understood that the terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its use in describing the present disclosure.

The articles “a” and “an” refer to one or to more than one (i.e., to at least one) of the grammatical object of the article unless the context clearly indicates otherwise. By way of example, “an element” means one element or more than one element.

### Immune Cell Receptor Modulators Conjugated to Immunoglobulin

Controlled tetra-Fc sialylation of immunoglobulin (IG) has been shown to have more potent anti-inflammatory action than unmodified IG in in vivo arthritis models. Sialyation of IG alters Fc glycosylation but requires complex enzymatic processes and results in heterogenous product.

As used herein, the term “antibody” refers to an immunoglobulin molecule that is typically composed of two identical pairs of polypeptide chains, each pair having one “light” (L) chain and one “heavy” (H) chain. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 3 or more amino acids. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The constant regions of the antibodies may mediate the binding of the immunoglobulin to host tissues or factors, including various cells of the immune system (e.g., effector cells) and the first component (C1q) of the classical complement system. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of each heavy/light chain pair (VH and VL), respectively, form the antibody binding site. The term “antibody” encompasses an antibody that is part of an antibody multimer (a multimeric form of antibodies), such as dimers, trimers, or higher-order multimers of monomeric antibodies. It also encompasses an antibody that is linked or attached to, or otherwise physically or functionally associated with, a non-antibody moiety. Further, the term “antibody” is not limited by any particular method of producing the antibody. For example, it includes, inter alia, recombinant antibodies, synthetic antibodies, monoclonal antibodies, polyclonal antibodies, bi-specific antibodies, and multi-specific antibodies.

As used herein, the term “antibody derivative” or “derivative” of an antibody refers to a molecule that is capable of binding to the same antigen that the antibody from which it is derived binds to and comprises an amino acid sequence that is the same or similar to the antibody linked to an additional molecular entity. The amino acid sequence of the antibody that is contained in the antibody derivative may be the full-length antibody, or may be any portion or portions of a full-length antibody. The additional molecular entity may be a chemical or biological molecule. Examples of additional molecular entities include chemical groups, amino acids, peptides, proteins (such as enzymes, antibodies), and chemical compounds. The additional molecular entity may have any utility, such as for use as a detection agent, label, marker, pharmaceutical or therapeutic agent. The amino acid sequence of an antibody may be attached or linked to the additional entity by chemical coupling, genetic fusion, noncovalent association or otherwise. The term “antibody derivative” also encompasses chimeric antibodies, humanized antibodies, and molecules that are derived from modifications of the amino acid sequences of an antibody, such as conservation amino acid substitutions, additions, and insertions.

As used herein, the term “antigen-binding fragment” of an antibody refers to one or more portions of a full-length antibody that retain the ability to bind to the same antigen that the antibody binds to.

As used herein, the terms “immunoglobulin,” “immune globulin,” “immunoglobulin molecule” and “IG” encompass (1) antibodies, (2) antigen-binding fragments of an antibody, and (3) derivatives of an antibody, each as defined herein. As described herein, immune globulin may be prepared from (e.g., fractionated from, isolated from, purified from, concentrated from, etc.) pooled plasma compositions (e.g., for administration to a subject). As used herein, the term “intravenous immune globulin (IVIG)” refers to conventional immunoglobulin prepared from the plasma of large numbers (e.g., 250, 500, 1000, or more) random human donors (e.g., HIZENTRA, Immune Globulin Subcutaneous, CSL Behring).

As used herein, the term “hyperimmune globulin” refers to immunoglobulin prepared from the plasma of donors with higher than normal titers of antibody (e.g., opsonic antibody) against a specific organism (e.g., S. pneumonia). For example, as used herein, an “anti-pneumococcal hyperimmune globulin” is an immunoglobulin containing a significantly elevated opsonic anti-pneumococcal antibody titer regardless of (e.g., that is independent and distinct from) the total titer or amount of antibody capable of binding S. pneumonia.

As used herein, the term “antibody sample” refers to an antibody-containing composition (e.g., fluid (e.g., plasma, blood, purified antibodies, blood or plasma fractions, blood or plasma components etc.)) taken from or provided by a donor (e.g., natural source) or obtained from a synthetic, recombinant, other in vitro source, or from a commercial source. The antibody sample may exhibit elevated titer of a particular antibody or set of antibodies based on the pathogenic/antigenic exposures (e.g., natural exposure or through vaccination) of the donor or the antibodies engineered to be produced in the synthetic, recombinant, or in vitro context. Herein, an antibody sample with elevated titer of antibody X is referred to as an “X-elevated antibody sample.” For example, an antibody sample with elevated titer of antibodies against S. pneumonia is referred to as a “S. pneumonia-elevated antibody sample.”

An aspect of the present disclosure relates to agents comprising conjugates of human plasma immunoglobulin and modulators of immune cell surface receptors (e.g., receptors implicated in pathophysiology of various disorders and diseases, for example, inflammatory, autoimmune, and oncological disorders). Compositions comprising conjugates of IG and one or more immune cell surface receptor binding moieties (e.g., modulators that enhance or dampen Fc receptor mediated immune responses) disclosed herein provide highly effective/potent anti-inflammatory properties (e.g., equal to or greater than tetra-Fc sialyated IG). In another aspect, a composition comprising conjugates of IG and one or more immune cell surface receptor binding moieties provides superior anti-inflammatory activity/properties compared to a non-conjugated IG (IG not conjugated to one or more immune cell surface receptor binding moieties disclosed herein). In still further aspects, a composition comprising conjugates of IG and one or more immune cell surface receptor binding moieties provides enhanced modulation of cytokine expression and/or activity compared to a non-conjugated IG (IG not conjugated to one or more immune cell surface receptor binding moieties disclosed herein). In addition, methods of manufacturing conjugates and/or agents disclosed herein are more homogeneous, scalable, and efficient than sialylation methodology.

The immune cell surface receptors, according to embodiments of the present disclosure, include Fc receptors that are expressed on the surface of of certain cells—including, among others, innate and adaptive immune cells, B lymphocytes, plasma cells, follicular dendritic cells, natural killer cells, macrophages, neutrophils, eosinophils, basophils, human platelets, and mast cells—that contribute to the protective functions of the immune system. In an embodiment, Fc receptors may be expressed on the surface of the natural killer cells. Such Fc receptors may include, but are not limited to, CD16A, CD32B, IL-15R, NKG2D, NKp46, 4-1BB, or any combination thereof. For review of the NK cells and associated receptors, please see, for example, Sivori S. et al. “Human NK cells: surface receptros, inhibitory checkpoints, and translational applications” Cellular & Molecular Immunology 2019 16:430-441, which is incorporated herein in its entirety by reference.

In some embodiments, the present disclosure provides technologies, e.g., mAb therapy enhancer (MATE™) technologies, that enable efficient site-directed chemical conjugation to an antibody agent (for example, immunoglobulin obtained from human plasma or immunoglobulin obtained from hyperimmune human plasma (e.g., plasma from vaccinated plasma donors or plasma from donors that have recovered from an infection)), and allow development of various multispecific therapeutic agents.

In some embodiments, advantages of provided technologies include site-directed conjugation specificity compared to certain existing methods that lack site-directed conjugation specificity by indiscriminately binding/conjugating to available amino acid residues. Aspects of the MATE technology have been described, for example, in WO 2021/102052, which is incorporated herein in its entirety by reference.

In an embodiment, an agent may include:
a human plasma immunoglobulin moiety,
an immune cell surface receptor binding moiety capable of modulating the immune cell surface receptor, and
optionally a linker moiety linking the human plasma immunoglobulin moiety and the immune cell surface receptor binding moiety.

The disclosure is not limited by the immune cell surface receptor binding moiety conjugated to the human plasma immunoglobulin moiety. Indeed, the immune cell surface receptor binding moiety may be any moiety that binds to and modulates (e.g., inhibits or augments) Fc receptor mediated immune responses including, but not limited to, moieties that modulate (e.g., inhibit or augment) Fc receptor mediated inflammatory responses and/or signaling and/or Fc receptor mediated cytokine responses and/or signaling. Examples of cell surface receptors to which a cell surface receptor binding moiety may bind include, but are not limited to, Fc receptors (e.g., that bind IgG antibodies) including, but not limited to, FcγRIIIa (CD16a), FcγRIIIb (CD16b), FcγRII (CD32), FcγRIIb (CD32B), and FcγRIIc (CD32C), as well as C-type lectin-like receptors such as, but not limited to, NKG2D.

In another embodiment, the agent may have the structure of formula M-1:

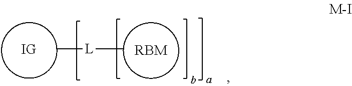

or a pharmaceutically acceptable salt thereof. In formula M-1:
a may be 1 or 2;
b may be 1, 2, or 3;
IG may be a human plasma immunoglobulin moiety;
each L may be a linker moiety; and
RBM may be an immune cell surface receptor binding moiety capable of modulating the immune cell surface receptor.

In some embodiments, RBM may be a peptide, a protein, an aptamer, a macromolecule (for example, an antibody) or a fragment thereof (for example, a single chain variable fragment scFV), or a small molecule, but are not limited thereto.

In an embodiment, the immunoglobulin moiety may include immunoglobulin, or fragments thereof, obtained from normal human plasma donors and/or immunoglobulin from an IVIG preparation (e.g., immunoglobulin derived from pooled human plasma from a multitude (e.g., 100, 200, 300, 400, 500, 600, 700, 800, 900, 1000 or more human plasma donors). The disclosure is not limited to any particular immune globulin (IG). Examples of IG include, but are not limited to, GAMMAGARD, CUVITRU, ASCENIV, PRIVIGEN, SYNAGIS, BIVIGAM, NABI-HB, or other IG available in the art. In some embodiments, the cell surface receptor may be CD16A (FcγRIIIa), CD32B (FcγRIIb), or NKG2D.

The immunoglobulin moiety may include antibodies of any isotype including IgG1 antibodies or fragments thereof, IgG2 antibodies or fragments thereof, or IgG4 antibodies or fragments thereof. For example, the immunoglobulin moiety may include IgG1 antibodies or fragments thereof linked to the linker L, at an amino acid residue selected from K246 and K248 of an IgG1 heavy chain and amino acid residues corresponding thereto. In another example, the immunoglobulin moiety may include IgG2 antibodies or fragments thereof linked to the linker, at an amino acid residue selected from K251 and K253 of an IgG2 heavy chain and amino acid residues corresponding thereto. In yet another example, the immunoglobulin moiety may include IgG4 antibodies or fragments thereof linked to the linker, at an amino acid residue selected from K239 and K241 of an IgG4 heavy chain and amino acid residues corresponding thereto.

In formula M-I,
L may be a covalent bond, or a bivalent or polyvalent optionally substituted, linear or branched C₁₋₁₀₀ group comprising one or more aliphatic, aryl, heteroaromatic having 1-20 heteroatoms, or any combinations thereof, wherein one or more methylene units of the group may be optionally and independently replaced with C₁₋₆ alkylene, C₁₋₆ alkenylene, —C≡C—, -Cy-, —C(R′)₂—, —O—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or -[(—O—C(R′)₂—C(R′)₂—)ₙ]-, wherein n is 1-20;
—Cy- may be independently an optionally substituted bivalent monocyclic, bicyclic or polycyclic group wherein each monocyclic ring may be independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
each R′ may be independently —R, —C(O)R, —CO₂R, or —SO₂R; and
each R may be independently —H, or an optionally substituted.

The linker may include one or more —[(CH₂)ₙ—O]ₘ—, wherein each n may be independently 1-20, and m may be 1-100.

Examples of CD16A modulators are known in the art and have been described, for example, in PCT Publication No. 2021/116277, which is incorporated herein in its entirety by reference.

Examples of CD32B modulators are known in the art and have been described, for example, in US 2011/0038869, US 2017/198040, and US 2021/0040205, each of which is incorporated herein in its entirety by reference.

Examples of NKG2D modulators are known in the art and have been described, for example, in US 2005/0158307, US 2006/280755, US 2012/0148581, US 2014/0248289, and US 2021/221894, each of which is incorporated herein in its entirety by reference.

In another embodiment, provided is a method of treating an acute or chronic inflammatory disorder in a patient in need thereof, the method including administering to the patient a pharmaceutically effective amount of any of the agents disclosed herein. For example, the disorder may be acute respiratory distress syndrome (ARDS).

In still another embodiment, provided is a method of treating an acute or chronic autoimmune disorder in a patient in need thereof, the method including administering to the patient a pharmaceutically effective amount of any of the agents disclosed herein.

In yet another embodiment, provided is a method of treating cancer in a patient in need thereof, the method including administering to the patient a pharmaceutically effective amount of any of the agents disclosed herein.

In yet another embodiment, provided is a method of prophylactically and/or therapeutically treating infection (e.g., caused by a microbial pathogen) in a patient in need thereof, the method including administering to the patient a pharmaceutically effective amount of any of the agents disclosed herein.

### Immune Cell Receptor Modulators Conjugated to Hyperimmune Globulin

C-type lectin dendritic cell-specific intercellular adhesion molecule 3-grabbing nonintegrin (DC-SIGN), also known as (CD209/CLEC4L) is a type II Fc cell surface receptor that can serve as a docking site for pathogens on the surface of dendritic cells and macrophages. See, for example, Anthony R. M. “Identification of a receptor required for the anti-inflammatory activity of IVIG” PNAS 2008, 105(50), 19571-19578. The receptor can recognize glycans expressed by a range of different viruses to promote attachment and infection, as well as the capture and sequestration of virus, which may then be passed on to other permissive cells. It is known as an authentic endocytic receptor for influenza A virus entry and infection.

DC-SIGN also functions as an adhesion receptor through binding of intercellular adhesion molecule 2 (ICAM2) and intercellular adhesion molecule 3 (ICAM3). DC-SIGN binds to ICAM3 bringing increasing interactions between resting T cells to dendritic cells, and leads to a Th2 response and inflammation which can be more damaging than infection.

An aspect of the present disclosure relates to agents comprising conjugates of hyperimmune globulin and immune cell surface receptors for enhanced antiviral and/or anti-inflammatory properties.

The disclosure is not limited to any particular hyperimmune globulin. For example, an immunoglobulin moiety may be any hyperimmune globulin or fragments thereof, obtained from hyperimmune human plasma donors (e.g., donors that have been vaccinated and/or recovered from an infectious disease).

In an embodiment, an agent may include:
a hyperimmune globulin moiety,
an immune cell surface receptor binding moiety capable of modulating the cell surface receptor, and
optionally a linker moiety linking the hyperimmune globulin moiety and the immune cell surface receptor binding moiety.

In another embodiment, the agent may have the structure of formula M-II:

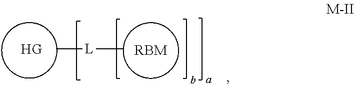

or a pharmaceutically acceptable salt thereof. In formula M-II:
a may be 1 or 2;
b may be 1, 2, or 3;
HG may be a hyperimmune globulin moiety;
each L may be a linker moiety; and
RBM may be an immune cell surface receptor binding moiety capable of modulating the immune cell surface receptor.

In some embodiments, RBM may be a peptide, a protein, an aptamer, a macromolecule (for example, an antibody) or a fragment thereof (for example, a single chain variable fragment scFV), or a small molecule, but are not limited thereto.

The cell surface receptor may be CD16A, CD32B, or NKG2D, or DC-SIGN.

The hyperimmune globulin moiety may include HIgG1 or a fragment thereof, HIgG2 or a fragment thereof, or HIgG4 or a fragment thereof. For example, the hyperimmune globulin moiety may include HIgG1 or a fragment thereof that is linked to the linker L, at an amino acid residue selected from K246 and K248 of an HIgG1 heavy chain and amino acid residues corresponding thereto. In another example, the hyperimmune globulin moiety may include HIgG2 or a fragment thereof that is linked to the linker, at an amino acid residue selected from K251 and K253 of an HIgG2 heavy chain and amino acid residues corresponding thereto. In yet another example, the hyperimmune globulin moiety may include IgG4 or a fragment thereof that is linked to the linker, at an amino acid residue selected from K239 and K241 of an IgG4 heavy chain and amino acid residues corresponding thereto.

The linker may be the same as described above for conjugates of a human plasma immunoglobulin and modulators of CD16, CD32, and NKG2.

### Reagents and Methods of Preparation

In another embodiment, provided is a method of treating an infectious disease in a patient in need thereof, the method including administering to the patient a pharmaceutically effective amount of any of the agents disclosed herein. In some aspects, one or more agents disclosed herein are utilized for administration to a subject in need thereof (e.g., for prophylactic administration and/or therapeutic treatment). For example, in an embodiment, one or more agents disclosed herein are utilized for passive immunization (e.g., immune-prophylaxis). In a further embodiment, one or more disclosed agents (e.g., a pharmaceutically effective amount of one or more disclosed agents) are administered to a subject to prophylactically treat infection associated with a microbial pathogen. In another embodiment, one or more disclosed agents (e.g., a pharmaceutically effective amount of one or more disclosed agents) are administered to a subject to therapeutically treat infection associated with a microbial pathogen. The disclosure is not limited to any particular infectious disease. In some embodiments, the infectious disease is a viral infection, for example, influenza or respiratory syncytial virus infection, or a bacterial infection, for example, S. pneumonia infection. Similarly, the disclosure is not limited to use of any particular agent described herein for treating and/or preventing infection (e.g., by a microbial pathogen). In some aspects, the agent comprises IG (e.g., immunoglobulin, or fragments thereof, obtained from normal human plasma donors, immunoglobulin, or fragments thereof, obtained from hyperimmune (e.g., vaccinated and/or recovered) human plasma donors, and/or immunoglobulin from an IVIG preparation) conjugated to one or more immune cell surface receptor binding moieties that enhance microbial pathogen clearance (e.g., moieties/modulators that inhibit microbial pathogen binding to host immune cells) and/or reduce host inflammatory responses (e.g., caused by T helper cell type 2 (Th2) cytokine expression). For example, in some embodiments, the disclosure provides an agent comprising IG (e.g., hyperimmune globulin) conjugated to an inhibitor moiety that blocks interactions between T cells and antigen presenting cells such as dendritic cells and macrophages. The disclosure is not limited to any particular inhibitor moiety. In some embodiments, the inhibitor moiety is a C-type lectin dendritic cell-specific intercellular adhesion molecule 3-grabbing nonintegrin (DC-SIGN) inhibitor.

As described herein, in some embodiments, the present disclosure provides technologies that can conjugate moieties of interest to targets (immune globulin) with high efficiency, high selectivity, and/or reduced side transformations (e.g., due to numbers of chemical reactions and/or conditions/types of chemical reactions). In some embodiments, the present disclosure provides useful reagents and methods for conjugation, and provide product compositions with enhanced homogeneity (e.g., 10%, 20%, 30%, 40%, 50%, 60%, 70%, 80%, 90%, 100%, or 2, 3, 4, 5, 6, 7, 8, 9, 10, 11, 12, 13, 14, 15, 16, 17, 18, 19, 20 or more fold, increase of modification/conjugation at one or more desired sites of target agents, and/or 10%, 20%, 30%, 40%, 50%, 60%, 70%, 80%, 90%, 100%, or 2, 3, 4, 5, 6, 7, 8, 9, 10, 11, 12, 13, 14, 15, 16, 17, 18, 19, 20 or more fold, decrease of modification/conjugation at one or more undesired sites of target agents), purity and/or reduced undesired modifications (e.g., to certain protein residues as results of side reactions). In some embodiments, the present disclosure provides a compound of formula R—I below or a salt thereof or a compound of formula R-II below or a salt thereof as described herein. In some embodiments, a compound of formula R—I or a salt thereof or a compound of formula R-II below or a salt thereof is useful for introducing a moiety of interest to a target in one step of reaction. In some embodiments, the present disclosure provides agents of formula P—I or P-II below, or a salt thereof. In some embodiments, a product composition comprise a plurality of agents having the structure of formula P—I or P-II, or a salt thereof, wherein the product composition has a higher level of homogeneity of said agents compared to a reference product composition (e.g., a product composition from a method in which a compound of formula R—I or a salt thereof is replaced with a compound which has the same structure as the compound of formula R—I or a salt thereof except that each target binding moiety is replaced with —H). In an embodiment, the target binding moiety may be a human plasma immunoglobulin or hyper immune globulin.

In some embodiments, the present disclosure provides a method, comprising steps of:
1) contacting a target agent, such as a human plasma immunoglobulin or hyperimmune globulin, with a reaction partner comprising:
   a first group comprising a target binding moiety that binds to a target agent,
   a reactive group;
   an immune cell surface receptor binding moiety capable of modulating the immune cell surface receptor; and
   optionally one or more linker moieties;
2) forming an agent comprising:
   a target agent moiety;
   an immune cell surface receptor binding moiety capable of modulating the immune cell surface receptor; and
   optionally one or more linker moieties.

In the method of this disclosure the target binding moiety binds specifically to the target agent and the reactive group reacts with specific sites of the target agent, such as specific lysine residues of a target agent antibody, such that the agent formed by the method comprises the immune cell surface receptor binding moiety attached, optionally via a linker, to the specific sites.

In some embodiments, a reaction group is located between a first group and a moiety of interest, and is connected to a first group and a moiety of interest independently and optionally through a linker moiety. In some embodiments, a reaction partner is a compound of formula R—I or a salt thereof or a compound of formula R-II or a salt thereof. In some embodiments, a first group is or comprises a LG^{IG} group as described herein. In some embodiments, a first group is or comprises a LG^{HG} group as described herein.

In some embodiments the disclosure provides a compound having the structure of formula R-L:

LG^{IG}-RG-L^{RM}-RBM, R—I

or a salt thereof, wherein:
LG^{IG} is a group comprising a target binding moiety that binds to a human plasma immunoglobulin with site-directed specificity,
RG is a reactive group;
L^{RM} is a linker; and
RBM is an immune cell surface receptor binding moiety capable of modulating the immune cell surface receptor. The cell surface receptor may be CD16, CD32, or NKG2.

In some embodiments the disclosure provides a compound, wherein the compound has the structure of formula R-L:

LG^{IG}-RG-L^{RM}-RBM, R—I

or a salt thereof, wherein:
LG^{IG} is R^{LG}-L^{LG};
R^{LG} is a moiety capable of binding to a human plasma immunoglobulin with site-directed specificity that is selected from

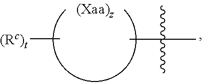

R^{c}-(Xaa)z-, IVIG, a nucleic acid moiety, and a small molecule moiety;
each Xaa is independently a residue of an amino acid or an amino acid analog;
t is 0-50;
z is 1-50;
each R^{c} is independently -L^{a}-R′;
each of a and b is independently 1-200;
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
L^{LG} is -L^{LG1}-, -L^{LG1}-L^{LG2}-, -L^{LG1}-L^{LG2}-L^{LG3}-, or -L^{LG1}-L^{LG2}-L^{LG3}-L^{LG4}-.
RG is -L^{RG1}-L^{RG2}-, -L^{LG4}-L^{RG1}-L^{RG2}-, -L^{LG3}-L^{LG4}-LRG-L^{RG2}-, -L^{LG2}-L^{LG3}-L^{LG4}-L^{RG1}-L^{RG2}-
each of L^{LG1}, L^{LG2}, L^{LG3}, L^{LG4}, L^{RG1}, L^{RG2}, and L^{RM} is independently L;
each L is independently a covalent bond, or a bivalent optionally substituted, linear or branched C₁₋₁₀₀ group comprising one or more aliphatic moieties, aryl moieties, heteroaliphatic moieties each independently having 1-20 heteroatoms, heteroaromatic moieties each independently having 1-20 heteroatoms, or any combinations of any one or more of such moieties, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or -[(—O—C(R′)₂—C(R′)₂—)ₙ]-, wherein n is 1-20;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms; and
RBM is an immune cell surface receptor binding moiety capable of modulating the immune cell surface receptor.

In some embodiments the disclosure provides a compound having the structure of formula R-II:

LG^{HG}-RG-L^{RM}-RBM, R-II

or a salt thereof, wherein:
LG^{HG} is a group comprising a target binding moiety that binds to hyperimmune globulin with site-directed specificity,
RG is a reactive group;
L^{RM} is a linker; and
RBM is an immune cell surface receptor binding moiety capable of modulating the immune cell surface receptor.

In some embodiments the disclosure provides a compound, wherein the compound has the structure of formula R-II:

LG^{HG}-RG-L^{RM}-RBM, R-II

or a salt thereof, wherein:
LG^{HG} is R^{LG}-L^{LG};
R^{LG} is a moiety capable of binding to a human plasma immunoglobulin with site-directed specificity that is selected from

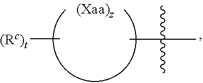

R^{c}-(Xaa)z-, IVIG, a nucleic acid moiety, and a small molecule moiety;
each Xaa is independently a residue of an amino acid or an amino acid analog;
t is 0-50;
z is 1-50;
each R^{c} is independently -L^{a}-R′;
each of a and b is independently 1-200;
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
L^{LG} is -L^{LG1}-, -L^{LG1}-L^{LG2}-, -L^{LG1}-L^{LG2}-L^{LG3}-, or -L^{LG1}-L^{LG2}-L^{LG3}-L^{LG4}-.
RG is -L^{RG1}-L^{RG2}-, -L^{LG4}-L^{RG1}-L^{RG2}-, -L^{LG3}-L^{LG4}-LRG-L^{RG2}-, -L^{LG2}-L^{LG3}-L^{LG4}-L^{RG1}-L^{RG2}-
each of L^{LG1}, L^{LG2}, L^{LG3}, L^{LG4}, L^{RG1}, L^{RG2}, and L^{RM} is independently L;
each L is independently a covalent bond, or a bivalent optionally substituted, linear or branched C₁₋₁₀₀ group comprising one or more aliphatic moieties, aryl moieties, heteroaliphatic moieties each independently having 1-20 heteroatoms, heteroaromatic moieties each independently having 1-20 heteroatoms, or any combinations of any one or more of such moieties, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or -[(—O—C(R′)₂—C(R′)₂—)ₙ]-, wherein n is 1-20;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms; and
RBM is an immune cell surface receptor binding moiety capable of modulating the immune cell surface receptor.

In some embodiments, the present disclosure provides a method comprising steps of:

1) contacting a target agent with a reaction partner having the structure of formula R-L:

LG^{IG}-RG-L^{RM}-RBM, R—I

or a salt thereof, wherein:
LG^{IG} is a group comprising a target binding domain that binds to a human plasma immunoglobulin with site-directed specificity,
RG is a reactive group;
L^{RM} is a linker; and
RBM is an immune cell surface receptor binding moiety capable of modulating the immune cell surface receptor; and

2) forming an agent having the structure of formula P-L:

P^{IG}-L^{PM}-RBM, P—I

or a salt thereof, wherein:
P^{IG} is a human plasma immunoglobulin moiety;
L^{PM} is a linker; and
RBM is an immune cell surface receptor binding moiety capable of modulating the cell surface receptor.

In some embodiments, the present disclosure provides a method comprising steps of:

1) contacting a target agent with a reaction partner having the structure of formula R-II:

LG^{HG}-RG-L^{RM}-RBM, R-II

or a salt thereof, wherein:
LG^{HG} is a group comprising a target binding domain that binds to hyperimmune globulin with site-directed specificity,
RG is a reactive group;
L^{RM} is a linker; and
RBM is an immune cell surface receptor binding moiety capable of modulating the immune cell surface receptor; and

2) forming an agent having the structure of formula P-II:

P^{HG}-L^{PM}-RBM, P-II

or a salt thereof, wherein:
P^{HG} is an hyperimmune globulin moiety;
L^{PM} is a linker; and
RBM is an immune cell surface receptor binding moiety capable of modulating the immune cell surface receptor.

In some embodiments, the present disclosure provides a method of manufacturing an agent having the structure of PN-II:

P^{IG}—N-L^{PM}-RBM, PN-II
wherein:
   P^{IG}—N is a human plasma immunoglobulin moiety comprising a lysine residue;
   L^{PM} is a linker; and
   RBM is an immune cell surface receptor binding moiety capable of modulating the immune cell surface receptor, wherein the immune cell surface receptor is CD16, CD32, or NKG2;
      the method comprising:
contacting P^{IG}—N with a reaction partner having a structure of formula R-L:

LG^{IG}-RG-L^{RM}-RBM, R—I

or a salt thereof, wherein:
LG^{IG} is a group comprising a human plasma immunoglobulin-binding domain that binds to P^{IG}—N with site-directed specificity,
RG is a reactive group;
L^{RM} is a linker; and
RBM is an immune cell surface receptor binding moiety capable of modulating the immune cell surface receptor.

In some embodiments, the present disclosure provides a method of manufacturing an agent having the structure of PN-II:

P^{HG}—N-L^{PM}-RBM, PN-II
wherein:
   P^{HG}—N is a human plasma immunoglobulin moiety comprising a lysine residue;
   L^{PM} is a linker; and
   RBM is an immune cell surface receptor binding moiety capable of modulating the cell surface receptor, wherein the immune cell surface receptor is DC-SIGN;
      the method comprising:
contacting P^{HG}—N with a reaction partner having a structure of formula R-II:

LG^{HG}-RG-L^{RM}-RBM, R-II

or a salt thereof, wherein:
LG^{HG} is a group comprising a hyperimmune globulin-binding domain that binds to P^{HG}—N with site-directed specificity,
RG is a reactive group;
L^{RM} is a linker; and
RBM is an immune cell surface receptor binding moiety capable of modulating the immune cell surface receptor.

In some embodiments, LG^{IG}/LG^{HG} is or comprises R^{LG}-L^{LG}-, wherein R^{LG} is or comprises a target binding moiety, and L^{L}G is L^{LG1} as described herein. In some embodiments, L^{LG} is -L^{LG1}-L^{LG2}-, wherein each of L^{LG1} and L^{LG2} is independently as described herein. In some embodiments, L^{LG} is -L^{LG1}-L^{LG2}-L^{LG3}-, wherein each of L^{LG1}, L^{LG2} and L^{LG3} is independently as described herein. In some embodiments, L^{LG} is -L^{LG1}-L^{LG2}-L^{LG3}-L^{LG4}-, wherein each of L^{LG1}, L^{LG2}, L^{LG3} and L^{LG4} is independently as described herein. In some embodiments, L^{LG1} is bonded to R^{L}G. In some embodiments, L^{LG1} is bonded to moiety of interest.

In some embodiments, L^{LG} is -L^{LG1}-, and a reactive group comprises L^{LG2}, L^{LG3} and L^{LG4}. In some embodiments, L^{LG} is -L^{LG1}-L^{LG2}-, and a reactive group comprises L^{LG3} and L^{LG4}. In some embodiments, L^{LG} is -L^{LG1}L^{LG2}-L^{LG3}-, and a reactive group comprises L^{LG4}.

In some embodiments, target binding moieties, first groups, and/or LG are released after reactions, e.g., after partner compounds react with target agents. In some embodiments, a first group is released after a reaction. In some embodiments, a target binding moiety is released after a reaction. In some embodiments, LG is released after a reaction. In some embodiments, a first group is released as part of a compound having the structure of LG-H or a salt thereof. In some embodiments, a target binding moiety is released as part of a compound having the structure of LG-H or a salt thereof. In some embodiments, LG is released as part of a compound having the structure of LG-H or a salt thereof.

In some embodiments, a first group is released as part of a compound having the structure of R^{LG}-L^{LG1}-L^{LG2}-L^{LG3}-L^{LG4}-H or a salt thereof. In some embodiments, a target binding moiety is released as part of a compound having the structure of R^{LG}-L^{LG1}-L^{LG2}-L^{LG3}-L^{LG4}-H or a salt thereof. In some embodiments, a target binding moiety is released as part of a compound having the structure of R^{LG}-L^{LG1}-L^{LG2}-L^{LG3}-L^{LG4}-H or a salt thereof, wherein R^{LG} is or comprises the target binding moiety. In some embodiments, LG is released as part of a compound having the structure of R^{LG}-L^{LG1}-L^{LG2}-L^{LG3}-L^{LG4}-H or a salt thereof, wherein LG is R^{LG}-L^{LG}, and L^{LG} is -L^{LG1}-, -L^{LG1}-L^{LG2}-, -L^{LG1}-L^{LG2}-L^{LG3}-, or -L^{LG1}-L^{LG2}-L^{LG3}-L^{LG4}-. In some embodiments, LG is released as part of a compound having the structure of R^{LG}-L^{LG1}-L^{LG2}-L^{LG3}-L^{LG4}-H or a salt thereof, wherein LG is R^{LG}-L^{LG1}-. In some embodiments, LG is released as part of a compound having the structure of R^{LG}-L^{LG1}-L^{LG2}-L^{LG3}-L^{LG4}-H or a salt thereof, wherein LG is R^{LG}-L^{LG1}-L^{LG2}. In some embodiments, LG is released as part of a compound having the structure of R^{L}G-L^{LG1}-L^{LG2}-L^{LG3}-L^{LG4}-H or a salt thereof, wherein LG is R^{LG}-L^{LG1}LL^{LG2}L^{G3}. In some embodiments, LG is released as part of a compound having the structure of R^{LG}-L^{LG1}-L^{LG2}-L^{LG3}-L^{LG4}-H or a salt thereof, wherein LG is R^{LG}-L^{LG1}-L^{LG2}-L^{LG3}-L^{LG4}.

In some embodiments, L is a covalent bond, or a bivalent optionally substituted, linear or branched C₁₋₁₀₀ group comprising one or more aliphatic moieties, aryl moieties, heteroaliphatic moieties each independently having 1-20 heteroatoms, heteroaromatic moieties each independently having 1-20 heteroatoms, or any combinations of any one or more of such moieties, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or -[(—O—C(R′)₂—C(R′)₂—)ₙ]-, wherein n is 1-20. In some embodiments, L is a covalent bond, or a bivalent optionally substituted, linear or branched C₁₋₁₀₀ aliphatic or heteroaliphatic group 1-20 heteroatoms, wherein one or more methylene units of the group are optionally and independently replaced with —C═C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, or -[(—O—C(R′)₂—C(R′)₂—)ₙ]-, wherein n is 1-20. In some embodiments, L is a covalent bond, or a bivalent optionally substituted, linear or branched C₁, C₂, C₃, C₄, C₅, C₁₀, C₁₅, C₂₀, C₂₅, C₃₀, C₄₀, C₅₀, C₆₀, C₁-2, C₁₋₅, C₁₋₁₀, C₁₋₁₅, C₁₋₂₀, C₁₋₃₀, C₁₋₄₀, C₁₋₅₀, C₁₋₆₀, C₁₋₇₀, C₁₋₈₀, or C₁₋₉₀ aliphatic or heteroaliphatic group 1-10 heteroatoms, wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, amino acid residues, or -[(—O—C(R′)₂—C(R′)₂—)ₙ]-, wherein n is 1-20. In some embodiments, L is a covalent bond, or a bivalent optionally substituted, linear or branched C₁, C₂, C₃, C₄, C₅, C₁₀, C₁₅, C₂₀, C₂₅, C₃₀, C₄₀, C₅₀, C₆₀, C₁-2, C₁₋₅, C₁₋₁₀, C₁₋₁₅, C₁₋₂₀, C₁₋₃₀, C₁₋₄₀, C₁₋₅₀, C₁₋₆₀, C₁₋₇₀, C₁₋₈₀, or C₁₋₉₀ aliphatic or heteroaliphatic group 1-10 heteroatoms, wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, amino acid residues, or -[(—O—C(R′)₂—C(R′)₂—)ₙ]-, wherein n is 1-10. In some embodiments, L is a covalent bond, or a bivalent optionally substituted, linear or branched C₁, C₂, C₃, C₄, C₅, C₁₀, C₁₅, C₂₀, C₂₅, C₃₀, C₄₀, C₅₀, C₆₀, C₁-2, C₁₋₅, C₁₋₁₀, C₁₋₁₅, C₁₋₂₀, C₁₋₃₀, C₁₋₄₀, C₁₋₅₀, C₁₋₆₀, C₁₋₇₀, C₁₋₈₀, or C₁₋₉₀ aliphatic group, wherein one or more methylene units of the group are optionally and independently replaced with —O—, —N(R′)—, —C(O)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, amino acid residues, or -[(—O—C(R′)₂—C(R′)₂—)ₙ]-, wherein n is 1-10. In some embodiments, L is a covalent bond, or a bivalent optionally substituted, linear or branched C₁, C₂, C₃, C₄, C₅, C₁₀, C₁₅, C₂₀, C₂₅, C₃₀, C₄₀, C₅₀, C₆₀, C₁-2, C₁₋₅, C₁₋₁₀, C₁₋₁₅, C₁₋₂₀, C₁₋₃₀, C₁₋₄₀, C₁₋₅₀, C₁₋₆₀, C₁₋₇₀, C₁₋₈₀, or C_{1- 90} aliphatic group, wherein one or more methylene units of the group are optionally and independently replaced with —O—, —N(R′)—, —C(O)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, or -[(—O—C(R′)₂—C(R′)₂-)ₙ]-, wherein n is 1-10. In some embodiments, L is a covalent bond, or a bivalent optionally substituted, linear or branched C₁₋₁₀ aliphatic group, wherein one or more methylene units of the group are optionally and independently replaced with —O—, —N(R′)—, —C(O)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, -Cy-, or -[(—O—C(R′)₂—C(R′)₂—)ₙ]-, wherein n is 1-10. In some embodiments, L is a covalent bond, or a bivalent optionally substituted, linear or branched C₁₋₁₀ aliphatic group, wherein one or more methylene units of the group are optionally and independently replaced with —O—, —N(R′)—, —C(O)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, or -[(—O—C(R′)₂—C(R′)₂-)ₙ]-, wherein n is 1-10. In some embodiments, L comprises no —C(O)O—. In some embodiments, L comprises no —C(O)—N(R′)—. In some embodiments, L comprises no —S—. In some embodiments, L comprises no —S-Cy-. In some embodiments, L comprises no —S—S—. In some embodiments, L does not contain one or more or any of —C(O)O—, —C(O)—N(R′)—, —S—, and —S—S—. In some embodiments, L does not contain one or more or any of —C(O)O—, —C(O)—N(R′)—, —S-Cy-, and —S—S—. In some embodiments, L does not contain one or more or any of —C(O)O—, —S—, and —S—S—. In some embodiments, L does not contain one or more or any of —C(O)O—, —S-Cy-, and —S—S—. In some embodiments, L contains none of —C(O)O—, —S—, and —S—S—. In some embodiments, L contains none of —C(O)O—, —S-Cy-, and —S—S—. In some embodiments, L contains none of —C(O)O— and —S—S—.

In some embodiments, each amino acid residue is independently a residue of an amino acid having the structure of formula A-I or a salt thereof. In some embodiments, each amino acid residue independently has the structure of —N(R^{a1})-L^{a1}-C(R^{a2})(R^{a3})-L^{a2}-CO— or a salt form thereof. In some embodiments, each amino acid residue independently has the structure of —N(R^{a1})—C(R^{a2})(R^{a3})—CO— or a salt form thereof.

In some embodiments, L is a covalent bond. In some embodiments, L is not a covalent bond.

In some embodiments, L^{LG1} is a covalent bond. In some embodiments, L^{LG1} is not a covalent bond. In some embodiments, L^{LG1} is or comprises —(CH₂CH₂O)ₙ—. In some embodiments, L^{LG1} is or comprises —(CH₂)ₙ—O—(CH₂CH₂O)ₙ—(CH₂)ₙ—, wherein each n is independently as described herein, and each —CH₂— is independently optionally substituted. In some embodiments, L^{LG1} is —(CH₂)ₙ—O—(CH₂CH₂O)ₙ—(CH₂)ₙ—, wherein each n is independently as described herein, and each —CH₂— is independently optionally substituted. In some embodiments, L^{LG1} is —(CH₂)₂—O—(CH₂CH₂O)ₙ—(CH₂)₂—, wherein n is as described herein, and each —CH₂— is independently optionally substituted. In some embodiments, L^{LG1} is —(CH₂)₂—O—(CH₂CH₂O)ₙ—(CH₂)₂—, wherein n is as described herein.

In some embodiments, L^{LG1} is —CH₂—. In some embodiments, L^{LG1} is —(CH₂)₂—. In some embodiments, L^{LG1} is —(CH₂)₂—C(O)—. In some embodiments, L^{LG1} is —(CH₂)₂—C(O)—NH—. In some embodiments, L^{LG1} is —(CH₂)₃—. In some embodiments, L^{LG1} is —(CH₂)₃NH—. In some embodiments, L^{LG1} is —(CH₂)₃NH—C(O)—. In some embodiments, L^{LG1} is —C(O)—(CH₂)₃NH—C(O)—. In some embodiments, L^{LG1} is —C(O)—(CH₂)₃—. In some embodiments, L^{LG1} is —NH—C(O)—(CH₂)₃—. In some embodiments, L^{LG1} is —NHC(O)—(CH₂)₃NH—C(O)—. In some embodiments, a —CH₂— is bonded to a target binding moiety.

In some embodiments, L^{LG1} is —CH₂CH₂—O—CH₂CH₂—O—CH₂CH₂—. In some embodiments, L^{LG1} is —CH₂CH₂—O—CH₂CH₂—O—CH₂CH₂—C(O)—. In some embodiments, L^{LG1} is —CH₂CH₂—O—CH₂CH₂—O—CH₂CH₂—C(O)NH—. In some embodiments, L^{L}d is —CH₂CH₂—O—CH₂CH₂—O—CH₂CH₂—C(O)NH—CH₂—. In some embodiments, —CH₂CH₂— is bonded to a target binding moiety.

In some embodiments, L^{LG1} is —(CH₂CH₂O)ₙ—. In some embodiments, L^{LG1} is —(CH₂CH₂O)ₙ—CH₂—CH₂—. In some embodiments, L^{LG1} is —(CH₂CH₂O)ₙ—CH₂—CH₂—C(O)—. In some embodiments, L^{LG1} is —(CH₂CH₂O)₂—CH₂—CH₂—C(O)—. In some embodiments, L^{LG1} is —(CH₂CH₂O)₄—CH₂—CH₂—C(O)—. In some embodiments, L^{LG1} is —(CH₂CH₂O)₈—CH₂—CH₂—C(O)—. In some embodiments, —C(O)— is bonded to a target binding moiety.

In some embodiments, L^{LG1} is —N(R′)—. In some embodiments, L^{LG1} is —NH—. In some embodiments, L^{LG1} is —NH—[(—CH₂CH₂—O-)]n-. In some embodiments, L^{LG1} is —NH—[(—CH₂CH₂—O-)]n-CH₂CH₂—. In some embodiments, L^{LG1} is —NH—[(—CH₂CH₂—O-)]n-CH₂CH₂—NH—. In some embodiments, L^{LG1} is —NH—[(—CH₂CH₂—O-)]n-CH₂CH₂—NH—C(O)—. In some embodiments, n is 1. In some embodiments, n is 2. In some embodiments, n is 3. In some embodiments, n is 4. In some embodiments, n is 5. In some embodiments, L^{LG1} is —NH—CH₂CH₂—O—. In some embodiments, L^{LG1} is —NH—CH₂CH₂—O—CH₂CH₂—. In some embodiments, L^{LG1} is —NH—CH₂CH₂—O—CH₂CH₂—NH—. In some embodiments, L^{LG1} is —NH—CH₂CH₂—O—CH₂CH₂—NH—C(O)—.

In some embodiments, L^{LG1} is —NH—[(—CH₂CH₂—O—)]₂-. In some embodiments, L^{LG1} is —NH—[(—CH₂CH₂—O—)]₂—CH₂CH₂—. In some embodiments, L^{LG1} is —NH—[(—CH₂CH₂—O—)]₂—CH₂CH₂—NH—. In some embodiments, L^{LG1} is —NH—[(—CH₂CH₂—O—)]₂—CH₂CH₂—NH—C(O)—.

In some embodiments, L^{LG1} is —NH—[(—CH₂CH₂—O—)]₃-. In some embodiments, L^{LG1} is —NH—[(—CH₂CH₂—O—)]₃—CH₂CH₂—. In some embodiments, L^{LG1} is —NH—[(—CH₂CH₂—O—)]₃—CH₂CH₂—NH—. In some embodiments, L^{LG1} is —NH—[(—CH₂CH₂—O—)]₃—CH₂CH₂—NH—C(O)—. In some embodiments, L^{LG1} is —NH—[(—CH₂CH₂—O—)]₄-. In some embodiments, L^{LG1} is —NH—[(—CH₂CH₂—O—)]₄—CH₂CH₂—. In some embodiments, L^{LG1} is —NH—[(—CH₂CH₂—O—)]₄—CH₂CH₂—NH—. In some embodiments, L^{LG1} is —NH—[(—CH₂CH₂—O—)]₄—CH₂CH₂—NH—C(O)—. In some embodiments, L^{LG1} is —NH—[(—CH₂CH₂—O—)]₅-. In some embodiments, L^{LG1} is —NH—[(—CH₂CH₂—O—)]₅—CH₂CH₂—. In some embodiments, L^{LG1} is —NH—[(—CH₂CH₂—O—)]₅—CH₂CH₂—NH—. In some embodiments, L^{LG1} is —NH—[(—CH₂CH₂—O—)]₅—CH₂CH₂—NH—C(O)—. In some embodiments, —NH— is bonded to a target binding moiety.

In some embodiments, L^{LG1} is —CH₂—. In some embodiments, L^{LG1} is —CH₂CH₂—. In some embodiments, L^{LG1} is —CH₂CH₂NH—. In some embodiments, L^{LG1} is —CH₂CH₂NH—(CO)—. In some embodiments, —CH₂— is bonded to a target binding moiety.

In some embodiments, L^{LG1} is —CH₂—. In some embodiments, L^{LG1} is —CH₂C(O)—. In some embodiments, L^{LG1} is —CH₂C(O)NH—. In some embodiments, L^{LG1} is —CH₂(CO)NHCH₂—. In some embodiments, —CH₂—C(O)— is bonded to a target binding moiety at —CH₂—.

In some embodiments, L^{LG2} is a covalent bond. In some embodiments, L^{LG2} is not a covalent bond. In some embodiments, L^{LG2} is —N(R′)C(O)—. In some embodiments, L^{LG2} is —NHC(O)—. In some embodiments, L^{LG2} is —(CH₂)ₙ—N(R′)C(O)—, wherein —(CH₂)ₙ— is optionally substituted. In some embodiments, L^{LG2} is —(CH₂)ₙ—OC(O)—, wherein —(CH₂)ₙ— is optionally substituted. In some embodiments, L^{LG2} is —(CH₂)ₙ—OC(O)N(R′)—, wherein —(CH₂)ₙ—is optionally substituted. In some embodiments, L^{LG2} is —(CH₂)ₙ—OC(O)NH—, wherein —(CH₂)ₙ—is optionally substituted. In some embodiments, n is 1-10, e.g., 1, 2, 3, 4, 5, 6, 7, 8, 9, or 10. In some embodiments, n is 1. In some embodiments, n is 2. In some embodiments, n is 3. In some embodiments, —(CH₂)ₙ—is substituted. In some embodiments, —(CH₂)ₙ—is unsubstituted. In some embodiments, L^{LG2} is —CH₂N(CH₂CH₂CH₂S(O)₂OH)—C(O)—. In some embodiments, L^{LG2} is —C(O)—NHCH₂—. In some embodiments, L^{LG2} is —C(O)—NHCH₂CH₂—. In some embodiments, L^{LG2} is —C(O)O—CH₂—. In some embodiments, L^{LG2} is —NH—C(O)O—CH₂—. In some embodiments, —C(O)— is bonded to L^{LG3}. In some embodiments, —N(R′)—, —NH—, or an optionally substituted —CH₂— unit (of optionally substituted —(CH₂)ₙ—) is bonded to L^{LG3}.

In some embodiments, L^{LG2} is —N(R′)—. In some embodiments, L^{LG2} is —N(R)—. In some embodiments, L^{LG2} is —NH—.

In some embodiments, L^{LG2} is optionally substituted bivalent C₁₋₆ aliphatic. In some embodiments, L^{LG2} is —CH₂—. In some embodiments, L^{LG2} is —CH₂NH—. In some embodiments, L^{LG2} is —CH₂NH—C(O)—. In some embodiments, L^{LG2} is —CH₂NH—C(O)—CH₂—.

In some embodiments, L^{LG3} is or comprises an optionally substituted aryl ring. In some embodiments, L^{LG3} is or comprises an optionally substituted phenyl ring. In some embodiments, L^{LG3} is a phenyl ring substituted with one or more electron-withdrawing groups. As appreciated by those skilled in the art, various electron-withdrawing groups are known in the art and may be utilized in accordance with the present disclosure. In some embodiments, an electron-withdrawing group is halogen. In some embodiments, an electron-withdrawing group is —F. In some embodiments, it is —Cl. In some embodiments, it is —Br. In some embodiments, it is —I. In some embodiments, an electron-withdrawing group comprises an X═Y double bond, wherein X is bonded to the group to which the electron-withdrawing group is a substituent, and at least one of X and Y is a heteroatom. In some embodiments, X is a heteroatom. In some embodiments, Y is a heteroatom. In some embodiments, each of X and Y is independently a heteroatom. In some embodiments, Y is O. In some embodiments, Y is S. In some embodiments, X is C. In some embodiments, X is N. In some embodiments, X is P. In some embodiments, X is S. In some embodiments, X═Y is C═O. In some embodiments, X═Y is N═O. In some embodiments, X═Y is S═O. In some embodiments, X═Y is P═O. In some embodiments, an electron-withdrawing group is —C(O)-L-R′. In some embodiments, an electron-withdrawing group is —C(O)—R′. In some embodiments, it is —NO₂. In some embodiments, it is —S(O)-L-R′. In some embodiments, it is —S(O)—R′. In some embodiments, it is —S(O)₂-L-R′. In some embodiments, it is —S(O)₂—O—R′. In some embodiments, it is —S(O)₂—N(R′)₂. In some embodiments, it is —P(O)(-L-R′)₂. In some embodiments, it is —P(O)(R′)₂. In some embodiments, it is —P(O)(OR′)₂. In some embodiments, it is —P(O)[N(R′)₂]₂.

In some embodiments, L^{LG3} is -L^{LG3a}-L^{LG3b}-, wherein L^{LG3a} is a covalent bond or —C(O)O—CH₂—, wherein —CH₂— is optionally substituted, and L^{LG3b} is an optionally substituted aryl ring. In some embodiments, L^{LG3a} is bonded to L^{LG2}, and L^{LG3b} is bonded to L^{LG4}.

In some embodiments, L^{LG3a} is a covalent bond. In some embodiments, L^{LG3a} is —C(O)O—CH₂—, wherein —CH₂— is optionally substituted. In some embodiments, L^{LG3a} is —C(O)O—CH₂—, wherein —CH₂— is substituted. In some embodiments, L^{LG3a} is —C(O)O—CH₂—, wherein —CH₂— is unsubstituted.

In some embodiments, a first group, a target binding moiety, and/or LG is released as part of a compound having the structure of R^{L}G-L^{LG1}-L^{LG2}-H or a salt thereof.

In some embodiments, L^{LG3b} is an optionally substituted phenyl ring. In some embodiments, at least one substituent is an electron-withdrawing group as described herein.

In some embodiments, L^{LG3} is

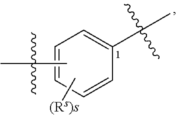

wherein s is 0-4, each R^{s} is independently halogen, —NO₂, -L-R′, —C(O)-L-R′, —S(O)-L-R′, —S(O)₂-L-R′, or —P(O)(-L-R′)₂. In some embodiments, C1 is bonded to L^{LG4}. In some embodiments, L^{LG3} is

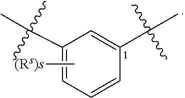

In some embodiments, L^{LG3} is

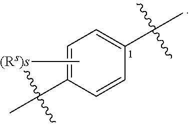

In some embodiments, L^{LG3} is

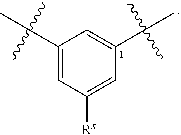

In some embodiments, L^{LG3} is

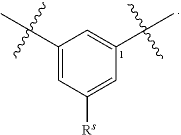

In some embodiments, L^{LG3} is

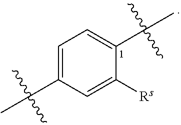

In some embodiments, L^{LG3} is

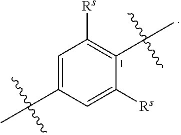

In some embodiments, L^{LG3b} is

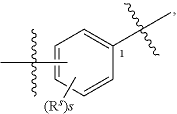

wherein s is 0-4, each R^{s} is independently halogen, —NO₂, -L-R′, —C(O)-L-R′, —S(O)-L-R′, —S(O)₂-L-R′, or —P(O)(-L-R′)₂. In some embodiments, C1 is bonded to L^{LG4}. In some embodiments, L^{LG3b} is

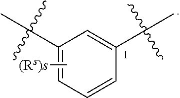

In some embodiments, L^{LG3b} is

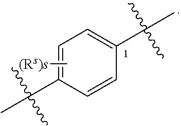

In some embodiments, L^{LG3b} is

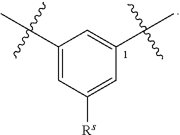

In some embodiments, L^{LG3b} is

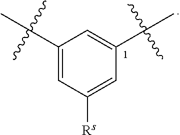

In some embodiments, L^{LG3b} is

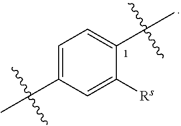

In some embodiments, L^{LG3b} is

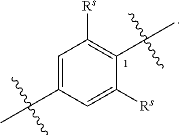

In some embodiments, s is 0. In some embodiments, s is 1-4. In some embodiments, s is 1. In some embodiments, s is 2. In some embodiments, s is 3. In some embodiments, s is 4.

In some embodiments, s is 1-4, and at least one R^{s} is an electron-withdrawing group, e.g., an electron-withdrawing group described above. In some embodiments, at least one R^{s} is —NO₂. In some embodiments, at least one R^{s} is —F. In some embodiments, each R^{s} is independently an electron-withdrawing group. In some embodiments, each R^{s} is —NO₂. In some embodiments, each R^{s} is —F.

In some embodiments, an electron-withdrawing group or R^{s} is at C2. In some embodiments, an electron-withdrawing group or R^{s} is at C3. In some embodiments, an electron-withdrawing group or R^{s} is at C4. In some embodiments, an electron-withdrawing group or R^{s} is at C2 and C5.

In some embodiments, L^{LG3} is

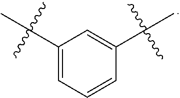 [SMILES: *c1cccc(*)c1]

In some embodiments, L^{LG3b} is

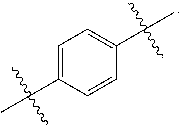 [SMILES: *c1ccc(*)cc1]

In some embodiments, L^{LG3} is

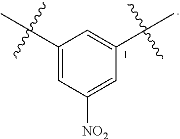 [SMILES: *c1cc(*)cc([N+](=O)[O-])c1]

In some embodiments, L^{LG3} is

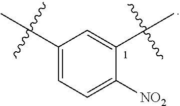 [SMILES: *c1ccc([N+](=O)[O-])c(*)c1]

In some embodiments, L^{LG3} is

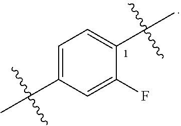 [SMILES: *c1ccc(*)c(F)c1]

In some embodiments, L^{LG3} is

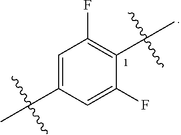 [SMILES: *c1cc(F)c(*)c(F)c1]

In some embodiments, L_{LG3} is

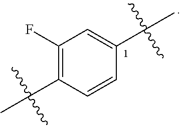 [SMILES: *c1ccc(*)c(F)c1]

In some embodiments, L^{LG3} is

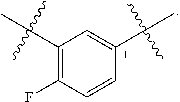 [SMILES: *c1ccc(F)c(*)c1]

In some embodiments, L^{LG3} b is

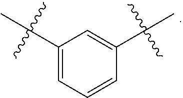 [SMILES: *c1cccc(*)c1]

In some embodiments, L^{LG3b} is

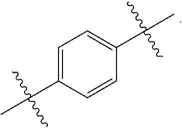 [SMILES: *c1ccc(*)cc1]

In some embodiments, L^{LG3b} is

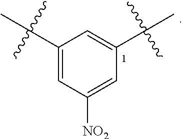 [SMILES: *c1cc(*)cc([N+](=O)[O-])c1]

In some embodiments, L^{LG3b} is

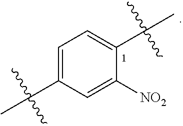 [SMILES: *c1ccc(*)c([N+](=O)[O-])c1]

In some embodiments, L^{LG3b} is

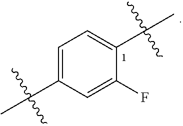 [SMILES: *c1ccc(*)c(F)c1]

In some embodiments, L^{LG3b} is

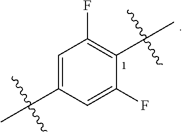 [SMILES: *c1cc(F)c(*)c(F)c1]

In some embodiments, L^{LG3b} is

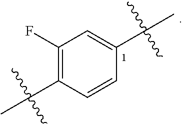 [SMILES: *c1ccc(*)c(F)c1]

In some embodiments, L^{LG3b} is

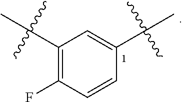 [SMILES: *c1ccc(F)c(*)c1]

In some embodiments, L^{LG3b} is optionally substituted

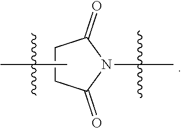

In some embodiments, the nitrogen atom is boned to L^{LG4} which is —O—. In some embodiments, the nitrogen atom is boned to L^{LG4} which is —O—, and -L^{RG1}-L^{RG2}- is —C(O)—.

In some embodiments, -L^{LG4}-L^{RG1}-L^{RG2}- is —O—C(O)—. In some embodiments, -L^{LG4}-L^{RG1}-L^{RG2}- is —S—C(O)—. In some embodiments, -L^{LG4}-L^{RG1}-L^{RG2}- is —S—C(O)—.

In some embodiments, L^{LG4} is a covalent bond. In some embodiments, L^{LG4} is not a covalent bond. In some embodiments, L^{LG4} is —O—. In some embodiments, L^{LG4} is —N(R′)—. In some embodiments, L^{LG4} is —NH—. In some embodiments, L^{LG4} is —N(CH₃)—. In some embodiments, L^{LG4} is —N(R′)—, and L^{LG3} is —O—. In some embodiments, R′ is optionally substituted C₁₋₆ alkyl. In some embodiments, L^{LG4} is —S—.

Various immunoglobulin/hyperimmune globulin binding moieties can be utilized in accordance with the present disclosure. Certain such binding moieties and technologies for identifying and/or assessing the binding moieties are described in WO 2019/023501 and WO 2019/136442, and are incorporated herein in their entireties by reference. Those skilled in the art appreciates that additional technologies in the art may be suitable for identifying and/or assessing antibody binding moieties in accordance with the present disclosure. In some embodiments, an immunoglobulin/hyperimmune globulin binding moiety comprises one or more amino acid residues, each independently natural or unnatural.

### Antibody-Binding Moiety

In some embodiments, a target binding moiety, e.g., a protein binding moiety (e.g., an antibody binding moiety (e.g., a universal antibody binding moiety)), has the structure of

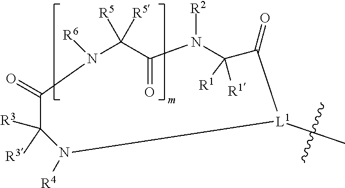

or a salt form thereof, wherein:
each of R¹, R³ and R⁶ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or:
   R¹ and R^{1′} are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur;
   R³ and R^{3′} are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur;
   an R⁵ group and the R^{5′} group attached to the same carbon atom are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur; or
   two R⁵ groups are optionally taken together with their intervening atoms to form a C₁₋₁₀ optionally substituted bivalent straight or branched saturated or unsaturated hydrocarbon chain wherein 1-3 methylene units of the chain are independently and optionally replaced with —S—, —SS—, —N(R)—, —O—, —C(O)—, —OC(O)—, —C(O)O—, —C(O)N(R)—, —N(R)C(O)—, —S(O)—, —S(O)₂—, or -Cy¹-, wherein each -Cy¹- is independently a 5-6 membered heteroarylenyl with 1-4 heteroatoms independently selected from nitrogen, oxygen or sulfur;
each of R^{1′}, R^{3′} and R^{5′} is independently hydrogen or optionally substituted C₁₋₃ aliphatic;
each of R², R⁴ and R⁶ is independently hydrogen, or optionally substituted C₁₋₄ aliphatic, or:
   R² and R¹ are optionally taken together with their intervening atoms to form a 4-8 membered, optionally substituted saturated or partially unsaturated monocyclic heterocyclic ring having 1-2 heteroatoms independently selected from nitrogen, oxygen, or sulfur;
   R⁴ and R³ are optionally taken together with their intervening atoms to form a 4-8 membered optionally substituted saturated or partially unsaturated monocyclic heterocyclic ring having 1-2 heteroatoms independently selected from nitrogen, oxygen, or sulfur; or
   an R⁶ group and its adjacent R⁵ group are optionally taken together with their intervening atoms to form a 4-8 membered optionally substituted saturated or partially unsaturated monocyclic heterocyclic ring having 1-2 heteroatoms independently selected from nitrogen, oxygen, or sulfur;
L¹ is a trivalent linker moiety; and
   each of m and n is independently 1, 2, 3, 4, 5, 6, 7, 8, 9, 10, 11, 12, 13, 14, 15, 16, 17, 18, 19, or 20.

In some embodiments, L¹ is an optionally substituted trivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, a target binding moiety, e.g. a protein binding moiety (e.g., an antibody binding moiety (e.g., a universal antibody binding moiety)), has the structure of

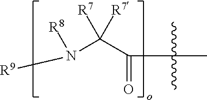

or a salt form thereof, wherein:
each of R⁷ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or:
   an R⁷ group and the R^{7′} group attached to the same carbon atom are optionally taken together with their intervening carbon atom to form a 3-8 membered optionally substituted saturated or partially unsaturated spirocyclic carbocyclic ring or a 3-8 membered optionally substituted saturated or partially unsaturated spirocyclic heterocyclic ring having 1-2 heteroatoms independently selected from nitrogen, oxygen, or sulfur;
each of R^{4′} is independently hydrogen or optionally substituted C₁₋₃ aliphatic;
each of R⁸ is independently hydrogen, or optionally substituted C₁₄ aliphatic, or:
   an R⁸ group and its adjacent R⁷ group are optionally taken together with their intervening atoms to form a 4-8 membered optionally substituted saturated or partially unsaturated monocyclic heterocyclic ring having 1-2 heteroatoms independently selected from nitrogen, oxygen, or sulfur; and
R⁹ is hydrogen, optionally substituted C₁₋₃ aliphatic, or —C(O)—.

In some embodiments, an antibody binding moiety, e.g., a universal antibody binding moiety is or comprises a peptide moiety, e.g., a moiety having the structure of R^{c}-(Xaa)z- or a salt form thereof, wherein each of R^{c}, z and Xaa is independently as described herein. In some embodiments, one or more Xaa are independently an unnatural amino acid residue. In some embodiments, side chains of two or more amino acid residues may be linked together to form bridges. For example, in some embodiments, side chains of two cysteine residues may form a disulfide bridge comprising —S—S— (which, as in many proteins, can be formed by two —SH groups).

In some embodiments, a target binding moiety, e.g. a protein binding moiety (e.g., an antibody binding moiety (e.g., a universal antibody binding moiety)), is or comprises a cyclic peptide moiety, e.g., a moiety having the structure of

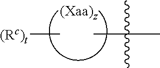

or a salt form thereof, wherein:
each Xaa is independently a residue of an amino acid or an amino acid analog;
t is 0-50;
z is 1-50;
L is a linker moiety;
each R^{c} is independently -L^{a}-R′;
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, a heteroatom is independently selected from oxygen, nitrogen, sulfur, phosphorus and silicon.

In some embodiments, a target binding moiety is or comprises R^{c}-(Xaa)z- or a salt form thereof, wherein each variable is as described herein. In some embodiments, a protein binding moiety is or comprises R^{c}-(Xaa)z- or a salt form thereof, wherein each variable is as described herein. In some embodiments, an antibody binding moiety, e.g., a universal antibody binding moiety, is or comprises R^{c}-(Xaa)z- or a salt form thereof, wherein each variable is as described herein. In some embodiments, a target binding moiety is or comprises

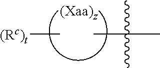

or a salt form thereof, wherein each variable is as described herein. In some embodiments, a protein binding moiety is or comprises

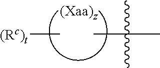

or a salt form thereof, wherein each variable is as described herein. In some embodiments, an antibody binding moiety, e.g., a universal antibody binding moiety, is or comprises

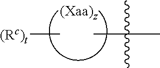

or a salt form thereof, wherein each variable is as described herein. In some embodiments, an antibody binding moiety, e.g., a universal antibody binding moiety is R^{c}-(Xaa)z- or

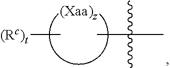

or a salt form thereof, and is or comprises a peptide unit. In some embodiments, -(Xaa)z- is or comprises a peptide unit. In some embodiments, amino acid residues may form bridges, e.g., connections formed by side chains optionally through linker moieties (e.g., L); for example, as in many polypeptides, cysteine residues may form disulfide bridges. In some embodiments, a peptide unit comprises an amino acid residue (e.g., at physiological pH about 7.4, “positively charged amino acid residue”, Xaa^{p}), e.g., a residue of an amino acid of formula A-I that has a positively charged side chain. In some embodiments, a peptide unit comprises R. In some embodiments, at least one Xaa is R. In some embodiments, a peptide unit is or comprises APAR. In some embodiments, a peptide unit is or comprises RAPA. In some embodiments, a peptide unit comprises an amino acid residue, e.g., a residue of an amino acid of formula A-I, that has a side chain comprising an aromatic group (“aromatic amino acid residue”, Xaa^{A}). In some embodiments, a peptide unit comprises a positively charged amino acid residue and an aromatic amino acid residue. In some embodiments, a peptide unit comprises W. In some embodiments, a peptide unit comprises a positively charged amino acid residue and an aromatic amino acid residue. In some embodiments, a peptide unit is or comprises Xaa^{A}XaaXaa^{P}Xaa^{P}. In some embodiments, a peptide unit is or comprises Xaa^{P}Xaa^{P}XaaXaa^{A}. In some embodiments, a peptide unit is or comprises Xaa^{P}Xaa^{A}Xaa^{P}. In some embodiments, a peptide unit is or comprises two or more Xaa^{P}Xaa^{A}Xaa^{P}. In some embodiments, a peptide unit is or comprises Xaa^{P}Xaa^{A}Xaa^{P}XaaXaa^{P}Xaa^{A}Xaa^{P}. In some embodiments, a peptide unit is or comprises Xaa^{P}Xaa^{P}Xaa^{A}Xaa^{A}Xaa^{P}. In some embodiments, a peptide unit is or comprises Xaa^{P}Xaa^{P}Xaa^{P}Xaa^{A}. In some embodiments, a peptide unit is or comprises two or more Xaa^{A}Xaa^{A}Xaa^{P}. In some embodiments, a peptide residue comprises one or more proline residues. In some embodiments, a peptide unit is or comprises HWRGWA. In some embodiments, a peptide unit is or comprises WGRR. In some embodiments, a peptide unit is or comprises RRGW. In some embodiments, a peptide unit is or comprises NKFRGKYK. In some embodiments, a peptide unit is or comprises NRFRGKYK. In some embodiments, a peptide unit is or comprises NARKFYK. In some embodiments, a peptide unit is or comprises NARKFYKG. In some embodiments, a peptide unit is or comprises HWRGWV. In some embodiments, a peptide unit is or comprises KHFRNKD. In some embodiments, a peptide unit comprises a positively charged amino acid residue, an aromatic amino acid residue, and an amino acid residue, e.g., a residue of an amino acid of formula A-I, that has a negatively charged side chain (e.g., at physiological pH about 7.4, “negatively charged amino acid residue”, Xaa^{N}). In some embodiments, a peptide unit comprises RHRFNKD. In some embodiments, a peptide unit is RHRFNKD. In some embodiments, a peptide unit comprises TY. In some embodiments, a peptide unit is TY. In some embodiments, a peptide unit comprises TYK. In some embodiments, a peptide unit is TYK. In some embodiments, a peptide unit comprises RTY. In some embodiments, a peptide unit is RTY. In some embodiments, a peptide unit comprises RTYK. In some embodiments, a peptide unit is RTYK. In some embodiments, a peptide unit is or comprises a sequence selected from PAM. In some embodiments, a peptide unit comprises WHL. In some embodiments, a peptide unit is WHL. In some embodiments, a peptide unit is or comprises WXL, wherein X is an amino acid residue as described herein, e.g., one suitable for connection with another moiety (e.g., an amino acid residue comprising —COOH or a salt or activated form thereof such as D, E, etc.). In some embodiments, a peptide unit comprises WDL. In some embodiments, a peptide unit is WDL. In some embodiments, a peptide unit comprises ELVW. In some embodiments, a peptide unit is ELVW. In some embodiments, a peptide unit comprises GELVW. In some embodiments, a peptide unit is GELVW. In some embodiments, a peptide unit is or comprises a sequence selected from AWHLGELVW. In some embodiments, a peptide unit is or comprises AWHLGELVW. In some embodiments, a peptide unit is or comprises a sequence selected from AWDLGELVW. In some embodiments, a peptide unit is or comprises AWDLGELVW. In some embodiments, a peptide unit is or comprises AWXLGELVW, wherein X is an amino acid residue as described herein, e.g., one suitable for connection with another moiety (e.g., an amino acid residue comprising —COOH or a salt or activated form thereof such as D, E, etc.). In some embodiments, a peptide unit is or comprises a sequence selected from DCAWHLGELVWCT, wherein the two cysteine residues can form a disulfide bond as found in natural proteins. In some embodiments, a peptide unit is or comprises DCAWHLGELVWCT, wherein the two cysteine residues can form a disulfide bond as found in natural proteins. In some embodiments, a peptide unit is or comprises a sequence selected from DCAWXLGELVWCT, wherein the two cysteine residues can form a disulfide bond as found in natural proteins, and X is an amino acid residue as described herein, e.g., one suitable for connection with another moiety (e.g., an amino acid residue comprising —COOH or a salt or activated form thereof such as D, E, etc.). In some embodiments, a peptide unit is or comprises DCAWXLGELVWCT, wherein the two cysteine residues can form a disulfide bond as found in natural proteins, and X is an amino acid residue as described herein, e.g., one suitable for connection with another moiety (e.g., an amino acid residue comprising —COOH or a salt or activated form thereof such as D, E, etc.). In some embodiments, X comprises —COOH or a salt or activated form thereof in its side chain. In some embodiments, a peptide unit is or comprises a sequence selected from DCAWDLGELVWCT, wherein the two cysteine residues can form a disulfide bond as found in natural proteins. In some embodiments, a peptide unit is or comprises DCAWDLGELVWCT, wherein the two cysteine residues can form a disulfide bond as found in natural proteins. In some embodiments, a peptide unit is or comprises a sequence selected from Fc-Ill. In some embodiments, a peptide unit is or comprises Fc-Ill. In some embodiments, a peptide unit is or comprises a sequence selected from DpLpAWXLGELVW, wherein X is an amino acid residue as described herein, e.g., one suitable for connection with another moiety (e.g., an amino acid residue comprising —COOH or a salt or activated form thereof such as D, E, etc.). In some embodiments, a peptide unit is or comprises DpLpAWXLGELVW, wherein X is an amino acid residue as described herein, e.g., one suitable for connection with another moiety (e.g., an amino acid residue comprising —COOH or a salt or activated form thereof such as D, E, etc.). In some embodiments, a peptide unit is or comprises a sequence selected from DpLpAWDLGELVW. In some embodiments, a peptide unit is or comprises DpLpAWDLGELVW. In some embodiments, a peptide unit is or comprises a sequence selected from DpLpAWHLGELVW, wherein the two cysteine residues can form a disulfide bond as found in natural proteins. In some embodiments, a peptide unit is or comprises DpLpAWHLGELVW (e.g., FcBP-1), wherein the two cysteine residues can form a disulfide bond as found in natural proteins. In some embodiments, a peptide unit is or comprises a sequence selected from FcBP-1. In some embodiments, a peptide unit is or comprises a sequence selected from DpLpDCAWXLGELVWCT, wherein the two cysteine residues can form a disulfide bond as found in natural proteins, and X is an amino acid residue as described herein, e.g., one suitable for connection with another moiety (e.g., an amino acid residue comprising —COOH or a salt or activated form thereof such as D, E, etc.). In some embodiments, a peptide unit is or comprises DpLpDCAWXLGELVWCT, wherein the two cysteine residues can form a disulfide bond as found in natural proteins, and X is an amino acid residue as described herein, e.g., one suitable for connection with another moiety (e.g., an amino acid residue comprising —COOH or a salt or activated form thereof such as D, E, etc.). In some embodiments, a peptide unit is or comprises a sequence selected from DpLpDCAWHLGELVWCT, wherein the two cysteine residues can form a disulfide bond as found in natural proteins. In some embodiments, a peptide unit is or comprises DpLpDCAWHLGELVWCT (e.g., FcBP-2), wherein the two cysteine residues can form a disulfide bond as found in natural proteins. In some embodiments, a peptide unit is or comprises a sequence selected from DpLpDCAWDLGELVWCT, wherein the two cysteine residues can form a disulfide bond as found in natural proteins. In some embodiments, a peptide unit is or comprises DpLpDCAWDLGELVWCT, wherein the two cysteine residues can form a disulfide bond as found in natural proteins. In some embodiments, a peptide unit is or comprises a sequence selected from FcBP-2. In some embodiments, a peptide unit is or comprises a sequence selected from CDCAWXLGELVWCTC, wherein the first and the last cysteines, and the two cysteines in the middle of the sequence, can each independently form a disulfide bond as in natural proteins, and X is an amino acid residue as described herein, e.g., one suitable for connection with another moiety (e.g., an amino acid residue comprising —COOH or a salt or activated form thereof such as D, E, etc.). In some embodiments, a peptide unit is or comprises CDCAWXLGELVWCTC, wherein the first and the last cysteines, and the two cysteines in the middle of the sequence, can each independently form a disulfide bond as in natural proteins, and X is an amino acid residue as described herein, e.g., one suitable for connection with another moiety (e.g., an amino acid residue comprising —COOH or a salt or activated form thereof such as D, E, etc.). In some embodiments, a peptide unit is or comprises a sequence selected from CDCAWHLGELVWCTC, wherein the first and the last cysteines, and the two cysteines in the middle of the sequence, can each independently form a disulfide bond as in natural proteins. In some embodiments, a peptide unit is or comprises CDCAWHLGELVWCTC, wherein the first and the last cysteines, and the two cysteines in the middle of the sequence, can each independently form a disulfide bond as in natural proteins. In some embodiments, a peptide unit is or comprises a sequence selected from CDCAWDLGELVWCTC, wherein the first and the last cysteines, and the two cysteines in the middle of the sequence, can each independently form a disulfide bond as in natural proteins. In some embodiments, a peptide unit is or comprises CDCAWDLGELVWCTC, wherein the first and the last cysteines, and the two cysteines in the middle of the sequence, can each independently form a disulfide bond as in natural proteins. In some embodiments, a peptide unit is or comprises a sequence selected from Fc-Ill-4c. In some embodiments, a peptide unit is or comprises a sequence selected from FcRM. In some embodiments, a peptide unit is or comprises a cyclic peptide unit. In some embodiments, a cyclic peptide unit comprises amide group formed by an amino group of a side chain and the C-terminus —COOH. It is appreciated by those skilled in the art that in various embodiments, when a peptide unit is connected to another moiety, an amino acid residue of a peptide unit may be connected through various positions, e.g., its backbone, its side chain, etc. In some embodiments, an amino acid residue is modified for connection. In some embodiments, an amino acid residue is replaced with another suitable residue for connection while maintaining one or more properties and/or activities a peptide unit (e.g., binding to an antibody as described herein). For example, in some embodiments, an amino acid residue is replaced with an amino acid residue with a side chain comprising —COOH or a salt or activated form thereof (e.g., side chain being —CH₂—COOH or a salt or activated form thereof). As exemplified herein, in various sequences H may be replaced with D (e.g., in various peptide units comprising WHL). In some embodiments, a peptide unit is connected to another moiety through —COOH or a salt or activated form thereof, e.g., through formation of e.g., —CON(R′)—. In some embodiments, R′ is —H. In some embodiments, —COOH is in a side chain of an amino acid residue. In some embodiments, in a sequence described herein (e.g., DCAWHLGELVWCT), 1-5 (e.g., 1, 2, 3, 4, or 5) amino acid residues may be independently and optionally replaced with another amino acid residue, 1-5 (e.g., 1, 2, 3, 4, or 5) amino acid residues may be independently and optionally deleted, and/or 1-5 (e.g., 1, 2, 3, 4, or 5) amino acid residues may be independently and optionally inserted. In some embodiments, a peptide moiety is connected to the rest of a molecule through its N-terminus. In some embodiments, it is connected to the rest of a molecule through its C-terminus. In some embodiments, it is connected to the rest of a molecule through a side chain of an amino acid residue (e.g., various X residues as described in the present disclosure). In some embodiments, two cysteine residues may independently and optionally form a disulfide bond. In some embodiments, the total number of replacement, deletion and insertion is no more than 10 (e.g., 0, or no more than 1, 2, 3, 4, 5, 6, 7, 8, 9, or 10). In some embodiments, the total number is 0. In some embodiments, the total number is no more than 1. In some embodiments, the total number is no more than 2. In some embodiments, the total number is no more than 3. In some embodiments, the total number is no more than 4. In some embodiments, the total number is no more than 5. In some embodiments, the total number is no more than 6. In some embodiments, the total number is no more than 7. In some embodiments, the total number is no more than 8. In some embodiments, the total number is no more than 9. In some embodiments, the total number is no more than 10. In some embodiments, there are no insertions. In some embodiments, there are no deletions.

In some embodiments, -(Xaa)z- is or comprises [X¹]ₚ₁[X²]ₚ₂—X³X⁴X⁵X⁶X⁷X⁸X⁹X¹⁰X¹¹X¹²—[X³]ₚ₁₃—[X¹⁴]ₚ₁₄[X¹⁵]ₚ₁₅[X¹⁶]ₚ₁₆, wherein each of X¹, X², X³, X⁴, X⁵, X⁶, X⁷, X⁸, X⁹, X¹⁰, X¹¹, X¹², and X¹³ is independently an amino acid residue, e.g., of an amino acid of formula A-I, and each of p1, p2, p13, p14, p15 and p16 is independently 0, 1, 2, 3, 4, 5, 6, 7, 8, 9, or 10. In some embodiments, each of X¹, X², X³, X⁴, X⁵, X⁶, X⁷, X⁸, X⁹, X¹⁰, X¹¹, X¹², and X¹³ is independently an amino acid residue of an amino acid of formula A-I. In some embodiments, each of X¹, X², X³, X⁴, X⁵, X⁶, X⁷, X⁸, X⁹, X¹⁰, X¹¹, X¹², and X¹³ is independently a natural amino acid residue. In some embodiments, one or more of X¹, X², X³, X⁴, X⁵, X⁶, X⁷, X⁸, X⁹, X¹⁰, X¹¹, X¹², and X¹³ are independently an unnatural amino acid residue as described in the present disclosure.

In some embodiments, a peptide unit comprises a functional group in an amino acid residue that can react with a functional group of another amino acid residue. In some embodiments, a peptide unit comprises an amino acid residue with a side chain which comprises a functional group that can react with another functional group of the side chain of another amino acid residue to form a linkage (e.g., see moieties described in Table A-1, example 8, etc.). In some embodiments, one functional group of one amino acid residue is connected to a functional group of another amino acid residue to form a linkage (or bridge). Linkages are bonded to backbone atoms of peptide units and comprise no backbone atoms. In some embodiments, a peptide unit comprises a linkage formed by two side chains of non-neighboring amino acid residues. In some embodiments, a linkage is bonded to two backbone atoms of two non-neighboring amino acid residues. In some embodiments, both backbone atoms bonded to a linkage are carbon atoms. In some embodiments, a linkage has the structure of L^{b}, wherein L^{b} is L^{a} as described in the present disclosure, wherein L^{a} is not a covalent bond. In some embodiments, L^{a} comprises -Cy-. In some embodiments, L^{a} comprises -Cy-, wherein -Cy- is optionally substituted heteroaryl. In some embodiments, -Cy- is

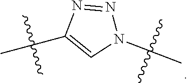 [SMILES: *c1cn(*)nn1]

In some embodiments, L^{a} is

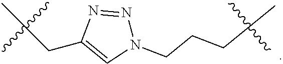 [SMILES: *CCCn1cc(C*)nn1]

In some embodiments, such an L^{a} can be formed by a —N₃ group of the side chain of one amino acid residue, and the -≡- of the side chain of another amino acid residue. In some embodiments, a linkage is formed through connection of two thiol groups, e.g., of two cysteine residues. In some embodiments, L^{a} comprises —S—S—. In some embodiments, L^{a} is —CH₂—S—S—CH₂—. In some embodiments, a linkage is formed through connection of an amino group (e.g., —NH₂ in the side chain of a lysine residue) and a carboxylic acid group (e.g., —COOH in the side chain of an aspartic acid or glutamic acid residue). In some embodiments, L^{a} comprises —C(O)—N(R′)—. In some embodiments, L^{a} comprise —C(O)—NH—. In some embodiments, L^{a} is —CH₂CONH—(CH₂)₃—. In some embodiments, L^{a} comprises —C(O)—N(R′)—, wherein R′ is R, and is taken together with an R group on the peptide backbone to form a ring (e.g., in A-34). In some embodiments, L^{a} is —(CH₂)₂—N(R′)—CO—(CH₂)₂—. In some embodiments, -Cy- is optionally substituted phenylene. In some embodiments, -Cy- is optionally substituted 1,2-phenylene. In some embodiments, L^{a} is

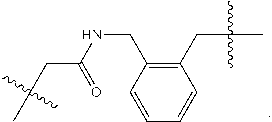 [SMILES: *CC(=O)NCc1ccccc1C*]

In some embodiments, L^{a} is

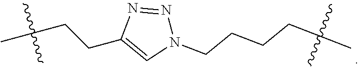 [SMILES: *CCCCn1cc(CC*)nn1]

In some embodiments, L^{a} is optionally substituted bivalent C₂₋₂₀ bivalent aliphatic. In some embodiments, L^{a} is optionally substituted —(CH₂)₉—CH═CH—(CH₂)₉—. In some embodiments, L^{a} is —(CH₂)₃—CH═CH—(CH₂)₃—.

In some embodiments, two amino acid residues bonded to a linkage are separated by 1, 2, 3, 4, 5, 6, 7, 8, 9, 10, 11, 12, 13, 14, 15, or more than 15 amino acid residues between them (excluding the two amino acid residues bonded to the linkage). In some embodiments, the number is 1. In some embodiments, the number is 2. In some embodiments, the number is 3. In some embodiments, the number is 4. In some embodiments, the number is 5. In some embodiments, the number is 6. In some embodiments, the number is 7. In some embodiments, the number is 8. In some embodiments, the number is 9. In some embodiments, the number is 10. In some embodiments, the number is 11. In some embodiments, the number is 12. In some embodiments, the number is 13. In some embodiments, the number is 14. In some embodiments, the number is 15.

In some embodiments, each of p1, p2, p13, p14, p15 and p16 is 0. In some embodiments, -(Xaa)z- is or comprises —X³X⁴X⁵X⁶X⁷X⁸X⁹X¹⁰X¹¹X¹²—, wherein:
each of X³, X⁴, X⁵, X⁶, X⁷, X⁸, X⁹, X¹⁰, X¹¹, and X¹² is independently an amino acid residue;
X⁶ is Xaa^{A} or Xaa^{P};
X⁹ is Xaa^{N}; and X¹² is Xaa^{A} or Xaa^{P}.

In some embodiments, each of X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, X⁵ is Xaa^{A} or Xaa^{P}. In some embodiments, X⁵ is Xaa^{A}. In some embodiments, X⁵ is Xaa^{P}. In some embodiments, X⁵ is an amino acid residue whose side chain comprises an optionally substituted saturated, partially saturated or aromatic ring. In some embodiments, X^{s} is

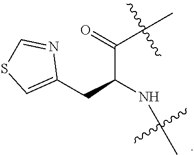 [SMILES: *N[C@@H](Cc1cscn1)C(*)=O]

In some embodiments, X⁵ is

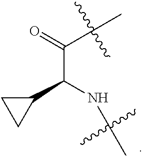 [SMILES: *N[C@H](C(*)=O)C1CC1]

In some embodiments, X⁶ is Xaa^{A}. In some embodiments, X⁶ is Xaa^{P}. In some embodiments, X⁶ is His. In some embodiments, X¹² is Xaa^{A}. In some embodiments, X¹² is Xaa^{P}. In some embodiments, X⁹ is Asp. In some embodiments, X⁹ is Glu. In some embodiments, X¹² is

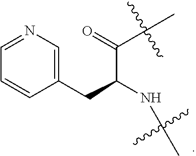 [SMILES: *N[C@@H](Cc1cccnc1)C(*)=O]

In some embodiments, X¹² is

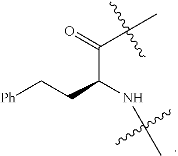 [SMILES: *N[C@@H](CCc1ccccc1)C(*)=O]

In some embodiments, each of X⁷, X¹⁰, and X¹¹ is independently an amino acid residue with a hydrophobic side chain (“hydrophobic amino acid residue”, Xaa^{H}). In some embodiments, X⁷ is Xaa^{H}. In some embodiments X⁷ is

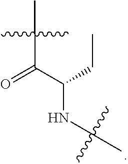 [SMILES: *N[C@@H](CC)C(*)=O]

In some embodiments, X⁷ is Val. In some embodiments, X¹⁰ is Xaa^{H}. In some embodiments, X¹⁰ is Met. In some embodiments, X¹⁰ is

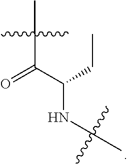 [SMILES: *N[C@@H](CC)C(*)=O]

In some embodiments, X¹¹ is Xaa^{H}. In some embodiments, X¹¹ is

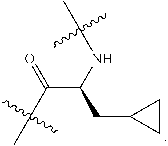 [SMILES: *N[C@@H](CC1CC1)C(*)=O]

In some embodiments, X⁸ is Gly. In some embodiments, X⁴ is Pro. In some embodiments, X³ is Lys. In some embodiments, the —COOH of X¹² forms an amide bond with the side chain amino group of Lys (X³), and the other amino group of the Lys (X³) is connected to a linker moiety and then a target binding moiety.

In some embodiments, -(Xaa)z- is or comprises —X³X⁴X⁵X⁶X⁷X⁸X⁹X¹⁰X¹¹X¹²—, wherein:
each of X³, X⁴, X⁵, X⁶, X⁷, X⁸, X⁹, X¹⁰, X¹¹, and X¹² is independently an amino acid residue;
at least two amino acid residues are connected through one or more linkages L^{b};
L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁶ is Xaa^{A} or Xaa^{P};
X⁹ is Xaa^{N}; and
X¹² is Xaa^{A} or Xaa^{P}.

In some embodiments, each of X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, X⁵ and X¹⁰ are connected by L^{b}. In some embodiments, there is one linkage L^{b}. In some embodiments, X⁶ is Xaa^{A}. In some embodiments, X⁶ is Xaa^{P}. In some embodiments, X⁶ is His. In some embodiments, X⁹ is Asp. In some embodiments, X⁹ is Glu. In some embodiments, X¹² is Xaa^{A}. In some embodiments, X¹² is

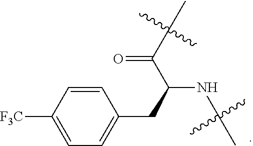 [SMILES: *N[C@@H](Cc1ccc(C(F)(F)F)cc1)C(*)=O]

In some embodiments, X¹² is

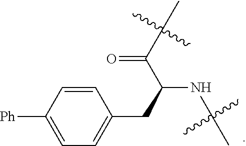 [SMILES: *N[C@@H](Cc1ccc(-c2ccccc2)cc1)C(*)=O]

In some embodiments, X¹² is

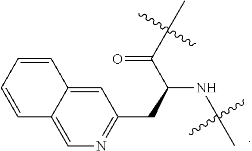 [SMILES: *N[C@@H](Cc1cc2ccccc2cn1)C(*)=O]

In some embodiments, each of X⁴, X⁷, and X¹¹ is independently Xaa^{H}. In some embodiments, X⁴ is Xaa^{H}. In some embodiments, X⁴ is Ala. In some embodiments, X⁷ is Xaa^{H}. In some embodiments, X⁷ is

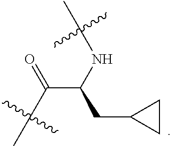 [SMILES: *N[C@@H](CC1CC1)C(*)=O]

In some embodiments, X¹¹ is Xaa^{H}. In some embodiments, X¹¹ is

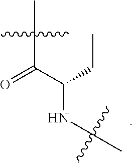 [SMILES: *N[C@@H](CC)C(*)=O]

In some embodiments, X⁸ is Gly. In some embodiments, X³ is Lys. In some embodiments, the —COOH of X¹² forms an amide bond with the side chain amino group of Lys (X³), and the other amino group of the Lys (X³) is connected to a linker moiety and then a target binding moiety. In some embodiments, L^{b} is

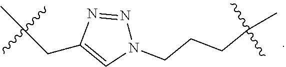 [SMILES: *CCCn1cc(C*)nn1]

In some embodiments, L^{b} is

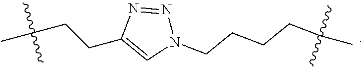 [SMILES: *CCCCn1cc(CC*)nn1]

In some embodiments, L^{b} connects two alpha-carbon atoms of two different amino acid residues. In some embodiments, both X⁵ and X¹⁰ are Cys, and the two —SH groups of their side chains form —S—S-(L^{b} is —CH₂—S—S—CH₂—).

In some embodiments, -(Xaa)z- is or comprises —X²X³X⁴X⁵X⁶X⁷X⁸X⁹X¹⁰X¹¹X¹²—, wherein:
each of X², X³, X⁴, X⁵, X⁶, X⁷, X⁸, X⁹, X¹⁰, X¹¹, and X¹² is independently an amino acid residue;
at least two amino acid residues are connected through one or more linkages L^{b};
L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁴ is Xaa^{A};
X⁵ is Xaa^{A} or Xaa^{P};
X⁸ is Xaa^{N}; and
X¹¹ is Xaa^{A}.

In some embodiments, each of X², X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, there is one linkage L^{b}. In some embodiments, X² and X¹² are connected by L^{b}. In some embodiments, L^{b} is —CH₂—S—S—CH₂—. In some embodiments, L^{b} is —CH₂—CH₂—S—CH₂—. In some embodiments, L^{b} is

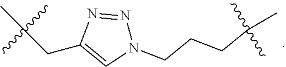 [SMILES: *CCCn1cc(C*)nn1]

In some embodiments, L^{b} is

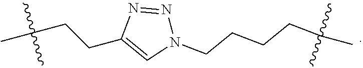 [SMILES: *CCCCn1cc(CC*)nn1]

In some embodiments, L^{b} is —CH₂CH₂CO—N(R′)—CH₂CH₂—. In some embodiments, R′ are taken together with an R group on the backbone atom that —N(R′)—CH₂CH₂—is bonded to form a ring, e.g., as in A-34. In some embodiments, a formed ring is 3-, 4-, 5-, 6-, 7- or 8-membered. In some embodiments, a formed ring is monocyclic. In some embodiments, a formed ring is saturated. In some embodiments, L^{b} is

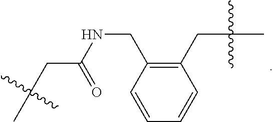 [SMILES: *CC(=O)NCc1ccccc1C*]

In some embodiments, L^{b} connects two alpha-carbon atoms of two different amino acid residues. In some embodiments, X⁴ is Xaa^{A}. In some embodiments, X⁴ is Tyr. In some embodiments, X⁵ is Xaa^{A}. In some embodiments, X⁵ is Xaa^{P}. In some embodiments, X⁵ is His. In some embodiments, X⁸ is Asp. In some embodiments, X⁸ is Glu. X¹¹ is Tyr. In some embodiments, both X² and X¹² are Cys, and the two —SH groups of their side chains form —S—S-(L^{b} is —CH₂—S—S—CH₂—). In some embodiments, each of X³, X⁶, X⁹, and X¹⁰ is independently Xaa^{H}. In some embodiments, X³ is Xaa^{H}. In some embodiments, X³ is Ala. In some embodiments, X⁶ is Xaa^{H}. In some embodiments, X⁶ is Leu. In some embodiments, X⁹ is Xaa^{H}. In some embodiments, X⁹ is Leu. In some embodiments, X⁹ is

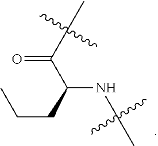 [SMILES: *N[C@@H](CCC)C(*)=O]

In some embodiments, X¹⁰ is Xaa^{H}. In some embodiments, X¹⁰ is Val. In some embodiments, X¹⁰ is

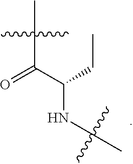 [SMILES: *N[C@@H](CC)C(*)=O]

In some embodiments, X⁷ is Gly. In some embodiments, p1 is 1. In some embodiments, X¹ is Asp. In some embodiments, p13 is 1. In some embodiments, p14, p15 and p16 are 0. In some embodiments, X¹³ is an amino acid residue comprising a polar uncharged side chain (e.g., at physiological pH, “polar uncharged amino acid residue”, Xaa^{t}). In some embodiments, X¹³ is Thr. In some embodiments, X¹³ is Val. In some embodiments, p13 is 0. In some embodiments, R^{c} is —NHCH₂CH(OH)CH₃. In some embodiments, R^{c} is (R)—NHCH₂CH(OH)CH₃. In some embodiments, R^{c} is (S)—NHCH₂CH(OH)CH₃.

In some embodiments, -(Xaa)z- is or comprises —X²X³X⁴X⁵X⁶X⁷X⁸X⁹X¹⁰X¹¹X¹²—, wherein:
each of X², X³, X⁴, X⁵, X⁶, X⁷, X⁸, X⁹, X¹⁰, X¹¹, and X¹² is independently an amino acid residue;
at least two amino acid residues are connected through one or more linkages L^{b};
L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁵ is Xaa^{A} or Xaa^{P};
X⁸ is Xaa^{N}; and
X¹¹ is Xaa^{A}.

In some embodiments, each of X², X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, there is one linkage L^{b}. In some embodiments, there are two or more linkages L^{b}. In some embodiments, there are two linkages L^{b}. In some embodiments, X² and X¹² are connected by L^{b}. In some embodiments, X⁴ and X⁹ are connected by L^{b}. In some embodiments, X⁴ and X¹⁰ are connected by L^{b}. In some embodiments, L^{b} is —CH₂—S—S—CH₂—. In some embodiments, L^{b} is

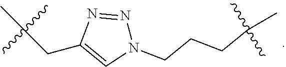 [SMILES: *CCCn1cc(C*)nn1]

In some embodiments, L^{b} is

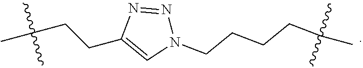 [SMILES: *CCCCn1cc(CC*)nn1]

In some embodiments, both X² and X¹² are Cys, and the two —SH groups of their side chains form —S—S-(L^{b} is —CH₂—S—S—CH₂—). In some embodiments, both X⁴ and X¹⁰ are Cys, and the two —SH groups of their side chains form —S—S-(L^{b} is —CH₂—S—S—CH₂—). In some embodiments, X⁴ and X⁹ are connected by L^{b}, wherein L^{b} is

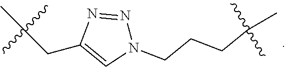 [SMILES: *CCCn1cc(C*)nn1]

In some embodiments, X⁴ and X⁹ are connected by L^{b}, wherein L^{b} is

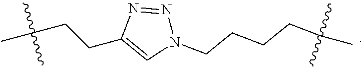 [SMILES: *CCCCn1cc(CC*)nn1]

In some embodiments, X⁵ is Xaa^{A}. In some embodiments, X⁵ is Xaa^{P}. In some embodiments, X⁵ is His. In some embodiments, X⁸ is Asp. In some embodiments, X⁸ is Glu. In some embodiments, X¹¹ is Tyr. In some embodiments, X¹¹ is

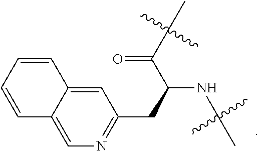 [SMILES: *N[C@@H](Cc1cc2ccccc2cn1)C(*)=O]

In some embodiments, X² and X¹² are connected by L^{b}, wherein L^{b} is —CH₂—S—CH₂CH₂—. In some embodiments, L^{b} connects two alpha-carbon atoms of two different amino acid residues. In some embodiments, each of X³, X⁶, and X⁹ is independently Xaa^{H}. In some embodiments, X³ is Xaa^{H}. In some embodiments, X³ is Ala. In some embodiments, X⁶ is Xaa^{H}. In some embodiments, X⁶ is Leu. In some embodiments, X⁶ is

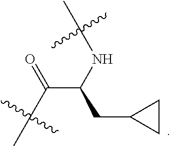 [SMILES: *N[C@@H](CC1CC1)C(*)=O]

In some embodiments, X⁹ is Xaa^{H}. In some embodiments, X⁹ is Leu. In some embodiments, X⁹ is

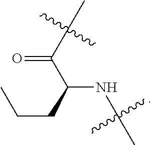 [SMILES: *N[C@@H](CCC)C(*)=O]

In some embodiments, X¹⁰ is Xaa^{H}. In some embodiments, X¹⁰ is Val. In some embodiments, X⁷ is Gly. In some embodiments, p1 is 1. In some embodiments, X¹ is Xaa^{N}. In some embodiments, X¹ is Asp. In some embodiments, X¹ is Glu. In some embodiments, p13 is 1. In some embodiments, p14, p15 and p16 are 0. In some embodiments, X¹³ is Xaa^{L}. In some embodiments, X¹³ is Thr. In some embodiments, X¹³ is Val.

In some embodiments, -(Xaa)z- is or comprises —X²X³X⁴X⁵X⁶X⁷X⁸X⁹X¹⁰X¹¹X¹²X¹³X¹⁴X¹⁵X¹⁶—, wherein:
each of X², X³, X⁴, X⁵, X⁶, X⁷, X⁸, X⁹, X¹⁰, X¹¹, X¹², X¹³, X¹⁴, X¹⁵, and X¹⁶ is independently an amino acid residue;
at least two amino acid residues are connected through a linkage L^{b};
L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X³ is Xaa^{N};
X⁶ is Xaa^{A};
X⁷ is Xaa^{A} or Xaa^{P};
X⁹ is Xaa^{N}; and
X¹³ is Xaa^{A}.

In some embodiments, each of X², X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, there is one linkage L^{b}. As appreciated by those skilled in the art, an amino acid residue may be replaced by another amino acid residue having similar properties, e.g., one Xaa^{H} (e.g., Val, Leu, etc.) may be replaced with another Xaa^{H} (e.g., Leu, lie, Ala, etc.), one Xaa^{A} may be replaced with another Xaa^{A}, one Xaa^{P} may be replaced with another Xaa^{P}, one Xaa^{N} may be replaced with another Xaa^{N}, one Xaa^{L} may be replaced with another Xaa^{L}, etc.

In some embodiments, a target binding moiety is or comprises optionally substituted moiety of Table A-1. In some embodiments, a protein binding moiety is or comprises optionally substituted moiety of Table A-1. In some embodiments, an antibody binding moiety, e.g., a universal antibody binding moiety, is or comprises optionally substituted moiety of Table A-1. In some embodiments, a target binding moiety is selected from able A-1. In some embodiments, a protein binding moiety is selected from able A-1. In some embodiments, an antibody binding moiety, e.g., a universal antibody binding moiety, is selected from able A-1. In some embodiments, C-terminus and/or N-terminus are optionally capped (e.g., for C-terminus, by converting —COOH into —C(O)N(R′)₂ like —C(O)NH₂; for N-terminus, by adding R′C(O)— like CH₃C(O)— to an amino group).

TABLE A-1
Exemplary antibody binding moieties.

| | |
|---|---|
| 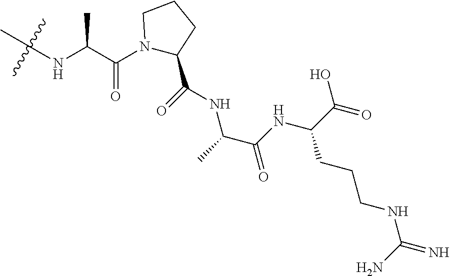 [SMILES: *N[C@@H](C)C(=O)N1CCC[C@H]1C(=O)N[C@@H](C)C(=O)N[C@@H](CCCNC(=N)N)C(=O)O] | A-1 |
| 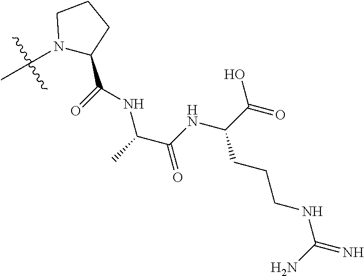 [SMILES: *N1CCC[C@H]1C(=O)N[C@@H](C)C(=O)N[C@@H](CCCNC(=N)N)C(=O)O] | A-2 |
| 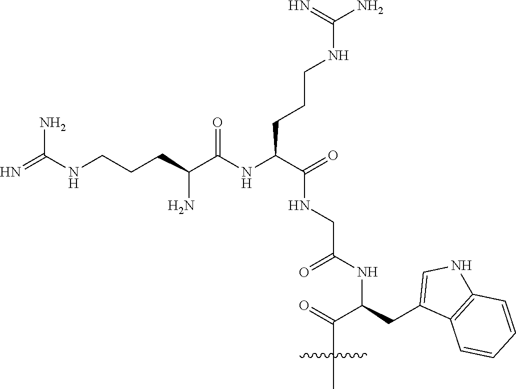 [SMILES: *C(=O)[C@H](Cc1c[nH]c2ccccc12)NC(=O)CNC(=O)[C@H](CCCNC(=N)N)NC(=O)[C@@H](N)CCCNC(=N)N] | A-3 |
| 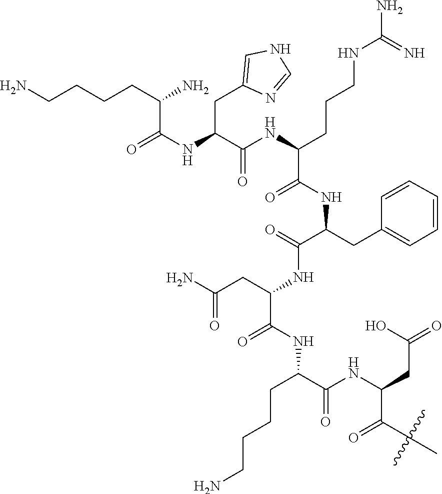 [SMILES: *C(=O)[C@H](CC(=O)O)NC(=O)[C@H](CCCCN)NC(=O)[C@H](CC(N)=O)NC(=O)[C@H](Cc1ccccc1)NC(=O)[C@H](CCCNC(=N)N)NC(=O)[C@H](Cc1c[nH]cn1)NC(=O)[C@@H](N)CCCCN] | A-4 |
| 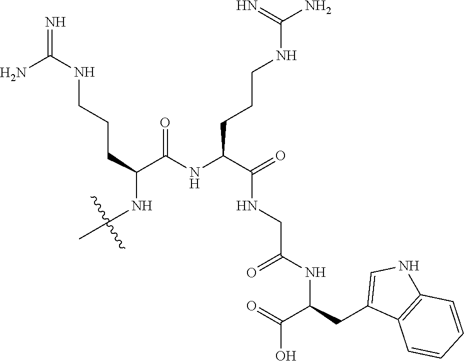 [SMILES: *N[C@@H](CCCNC(=N)N)C(=O)N[C@@H](CCCNC(=N)N)C(=O)NCC(=O)N[C@@H](Cc1c[nH]c2ccccc12)C(=O)O] | A-5 |
| 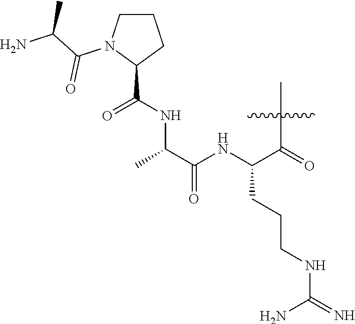 [SMILES: *C(=O)[C@H](CCCNC(=N)N)NC(=O)[C@H](C)NC(=O)[C@@H]1CCCN1C(=O)[C@H](C)N] | A-6 |
| 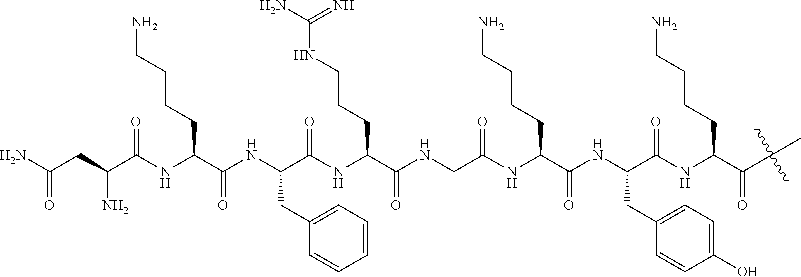 [SMILES: *C(=O)[C@H](CCCCN)NC(=O)[C@H](Cc1ccc(O)cc1)NC(=O)[C@H](CCCCN)NC(=O)CNC(=O)[C@H](CCCNC(=N)N)NC(=O)[C@H](Cc1ccccc1)NC(=O)[C@H](CCCCN)NC(=O)[C@@H](N)CC(N)=O] | A-7 |
| 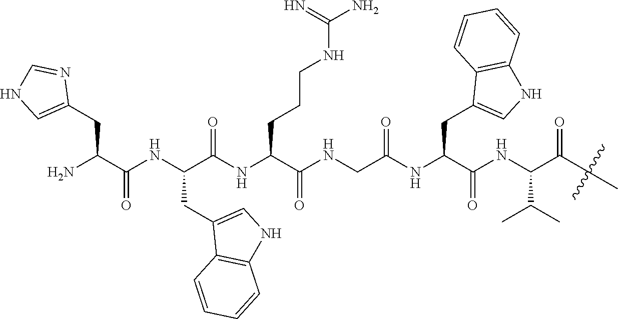 [SMILES: *C(=O)[C@@H](NC(=O)[C@H](Cc1c[nH]c2ccccc12)NC(=O)CNC(=O)[C@H](CCCNC(=N)N)NC(=O)[C@H](Cc1c[nH]c2ccccc12)NC(=O)[C@@H](N)Cc1c[nH]cn1)C(C)C] | A-8 |
| 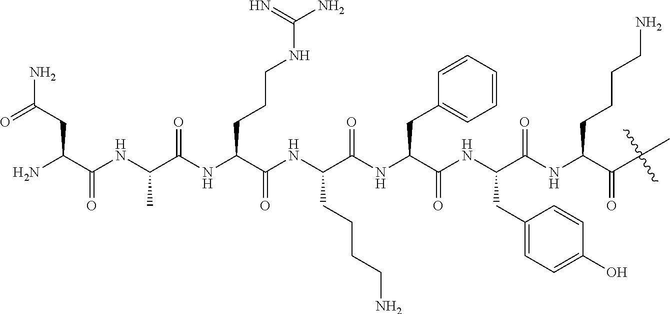 [SMILES: *C(=O)[C@H](CCCCN)NC(=O)[C@H](Cc1ccc(O)cc1)NC(=O)[C@H](Cc1ccccc1)NC(=O)[C@H](CCCCN)NC(=O)[C@H](CCCNC(=N)N)NC(=O)[C@H](C)NC(=O)[C@@H](N)CC(N)=O] | A-9 |
| 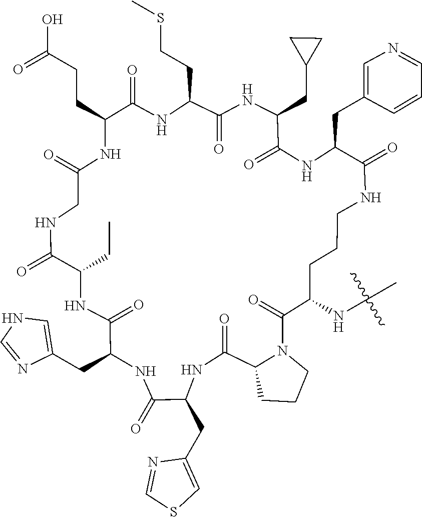 [SMILES: *N[C@H]1CCCNC(=O)[C@H](Cc2cccnc2)NC(=O)[C@H](CC2CC2)NC(=O)[C@H](CCSC)NC(=O)[C@H](CCC(=O)O)NC(=O)CNC(=O)[C@H](CC)NC(=O)[C@H](Cc2c[nH]cn2)NC(=O)[C@H](Cc2cscn2)NC(=O)[C@H]2CCCN2C1=O] | A-10 |
| 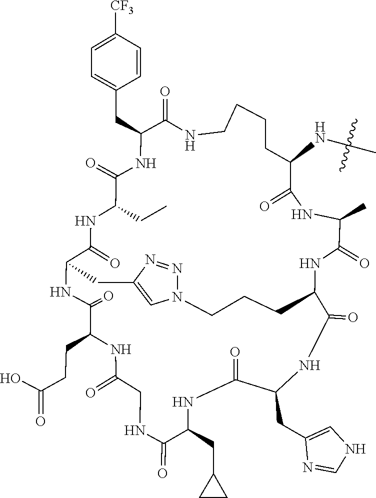 [SMILES: *N[C@@H]1CCCCNC(=O)[C@H](Cc2ccc(C(F)(F)F)cc2)NC(=O)[C@H](CC)NC(=O)[C@H]2Cc3cn(nn3)CCC[C@@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-11 |
| 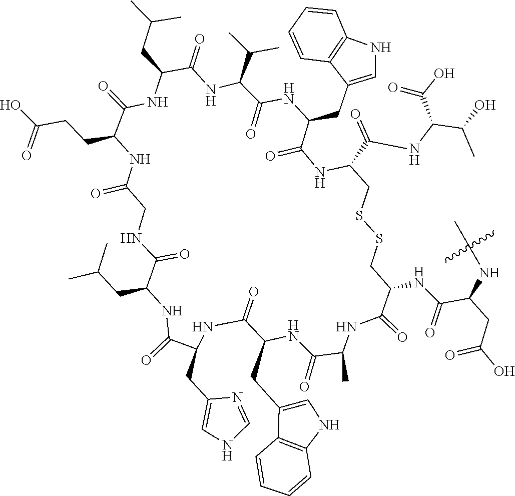 [SMILES: *N[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-12 |
| 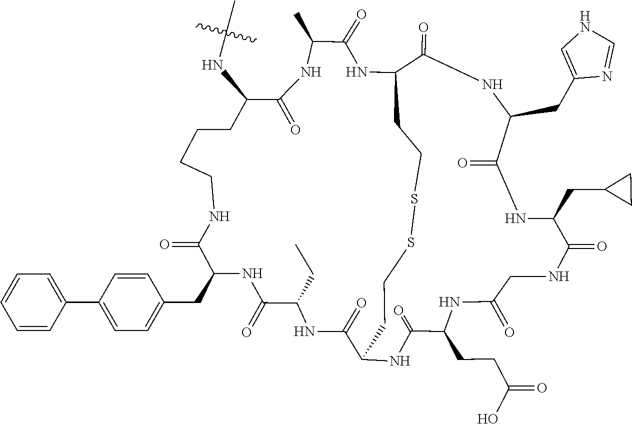 [SMILES: *N[C@@H]1CCCCNC(=O)[C@H](Cc2ccc(-c3ccccc3)cc2)NC(=O)[C@H](CC)NC(=O)[C@H]2CCSSCC[C@@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-13 |
| 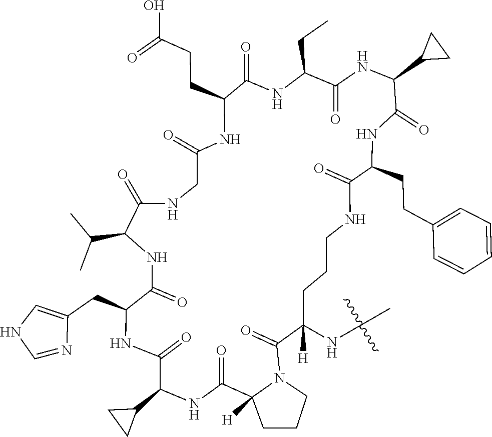 [SMILES: *N[C@H]1CCCNC(=O)[C@H](CCc2ccccc2)NC(=O)[C@H](C2CC2)NC(=O)[C@H](CC)NC(=O)[C@H](CCC(=O)O)NC(=O)CNC(=O)[C@H](C(C)C)NC(=O)[C@H](Cc2c[nH]cn2)NC(=O)[C@H](C2CC2)NC(=O)[C@H]2CCCN2C1=O] | A-14 |
| 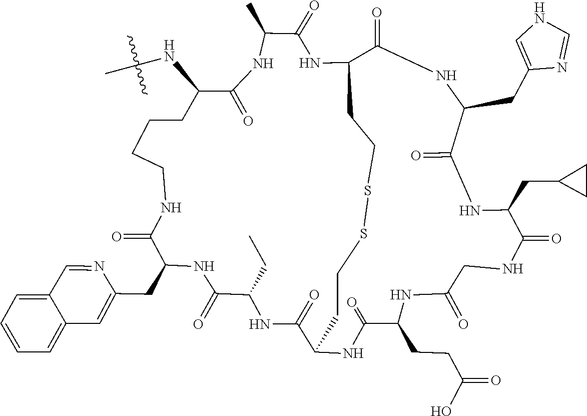 [SMILES: *N[C@@H]1CCCCNC(=O)[C@H](Cc2cc3ccccc3cn2)NC(=O)[C@H](CC)NC(=O)[C@H]2CCSSCC[C@@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-15 |
| 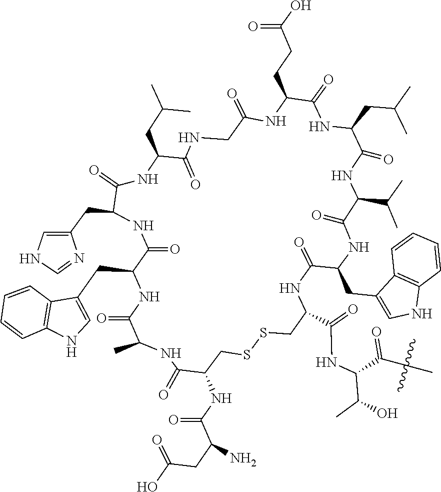 [SMILES: *C(=O)[C@@H](NC(=O)[C@@H]1CSSC[C@H](NC(=O)[C@@H](N)CC(=O)O)C(=O)N[C@@H](C)C(=O)N[C@@H](Cc2c[nH]c3ccccc23)C(=O)N[C@@H](Cc2c[nH]cn2)C(=O)N[C@@H](CC(C)C)C(=O)NCC(=O)N[C@@H](CCC(=O)O)C(=O)N[C@@H](CC(C)C)C(=O)N[C@@H](C(C)C)C(=O)N[C@@H](Cc2c[nH]c3ccccc23)C(=O)N1)[C@@H](C)O] | A-16 |
| 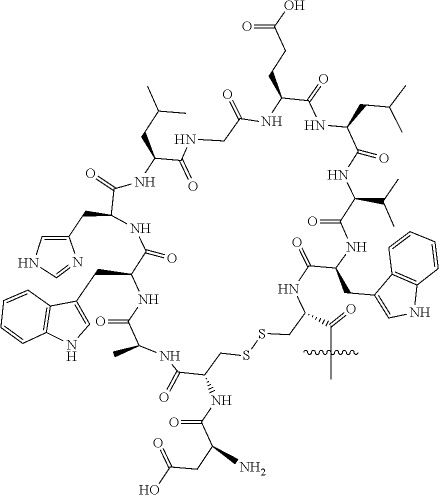 [SMILES: *C(=O)[C@@H]1CSSC[C@H](NC(=O)[C@@H](N)CC(=O)O)C(=O)N[C@@H](C)C(=O)N[C@@H](Cc2c[nH]c3ccccc23)C(=O)N[C@@H](Cc2c[nH]cn2)C(=O)N[C@@H](CC(C)C)C(=O)NCC(=O)N[C@@H](CCC(=O)O)C(=O)N[C@@H](CC(C)C)C(=O)N[C@@H](C(C)C)C(=O)N[C@@H](Cc2c[nH]c3ccccc23)C(=O)N1] | A-17 |
| 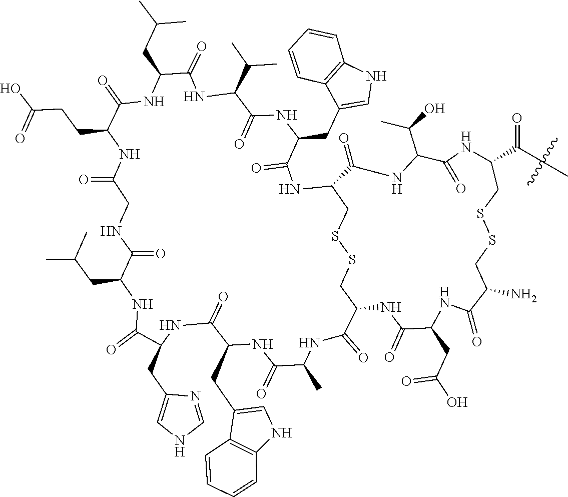 [SMILES: *C(=O)[C@@H]1CSSC[C@H](N)C(=O)N[C@@H](CC(=O)O)C(=O)N[C@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc3c[nH]cn3)NC(=O)[C@H](Cc3c[nH]c4ccccc34)NC(=O)[C@H](C)NC2=O)C(=O)NC([C@@H](C)O)C(=O)N1] | A-18 |
| 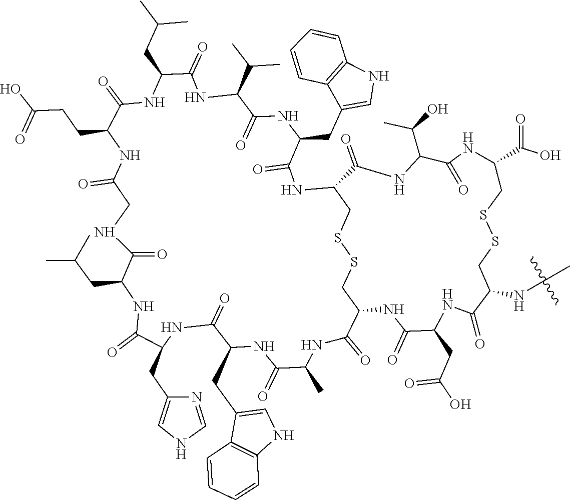 [SMILES: *N[C@H]1CSSC[C@@H](C(=O)O)NC(=O)C([C@@H](C)O)NC(=O)[C@@H]2CSSC[C@H](NC(=O)[C@H](CC(=O)O)NC1=O)C(=O)N[C@@H](C)C(=O)N[C@@H](Cc1c[nH]c3ccccc13)C(=O)N[C@@H](Cc1c[nH]cn1)C(=O)N[C@@H](CC(C)C)C(=O)NCC(=O)N[C@@H](CCC(=O)O)C(=O)N[C@@H](CC(C)C)C(=O)N[C@@H](C(C)C)C(=O)N[C@@H](Cc1c[nH]c3ccccc13)C(=O)N2] | A-19 |
| 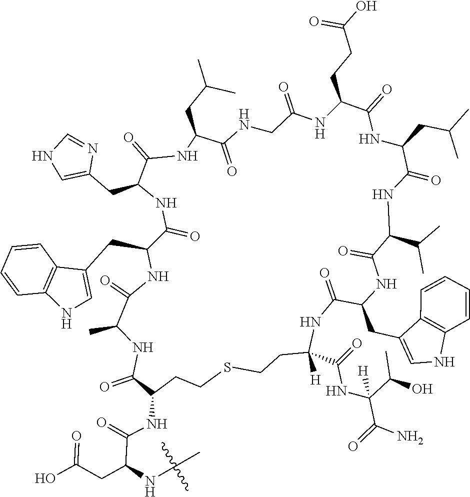 | A-20 |
| 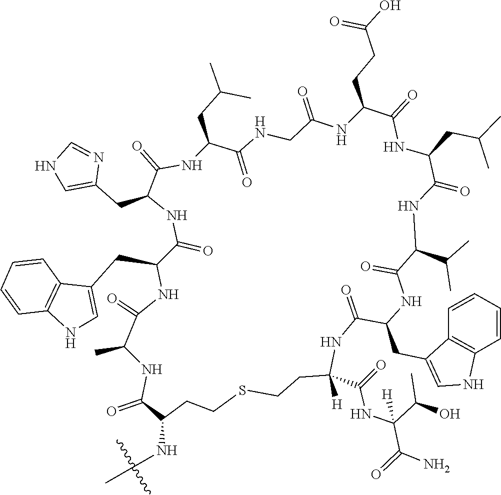 | A-21 |
| 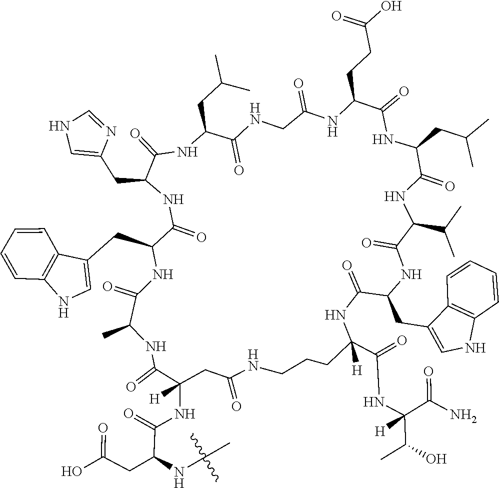 [SMILES: *N[C@@H](CC(=O)O)C(=O)N[C@H]1CC(=O)NCCC[C@@H](C(=O)N[C@H](C(N)=O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-22 |
| 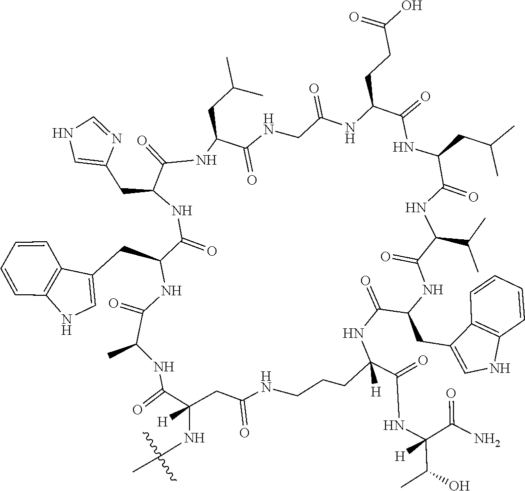 [SMILES: *N[C@H]1CC(=O)NCCC[C@@H](C(=O)N[C@H](C(N)=O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-23 |
| 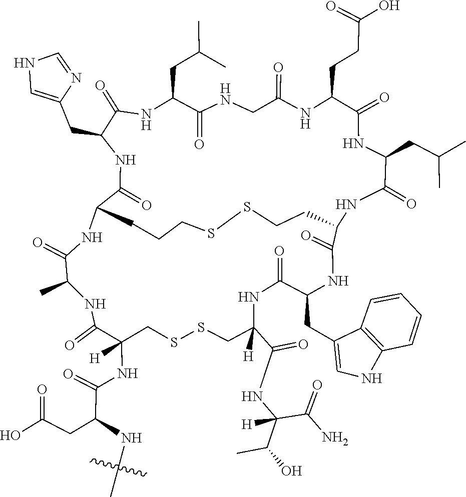 [SMILES: *N[C@@H](CC(=O)O)C(=O)N[C@H]1CSSC[C@@H](C(=O)N[C@H](C(N)=O)[C@@H](C)O)NC(=O)[C@H](Cc2c[nH]c3ccccc23)NC(=O)[C@@H]2CCSSCCC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CC(C)C)C(=O)N2] | A-24 |
| 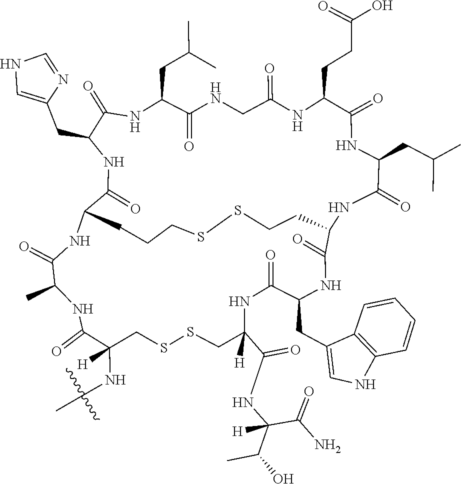 [SMILES: *N[C@H]1CSSC[C@@H](C(=O)N[C@H](C(N)=O)[C@@H](C)O)NC(=O)[C@H](Cc2c[nH]c3ccccc23)NC(=O)[C@@H]2CCSSCCC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CC(C)C)C(=O)N2] | A-25 |
| 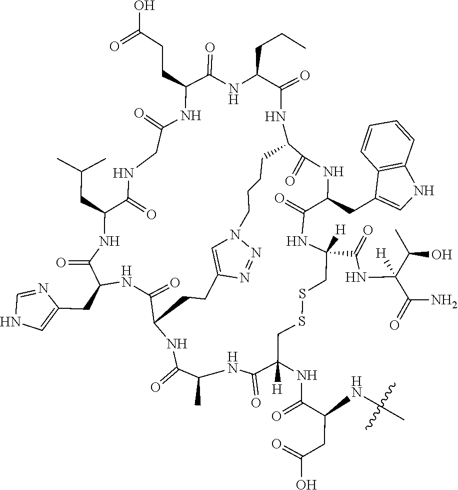 [SMILES: *N[C@@H](CC(=O)O)C(=O)N[C@H]1CSSC[C@@H](C(=O)N[C@H](C(N)=O)[C@@H](C)O)NC(=O)[C@H](Cc2c[nH]c3ccccc23)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CCC)C(=O)N2] | A-26 |
| 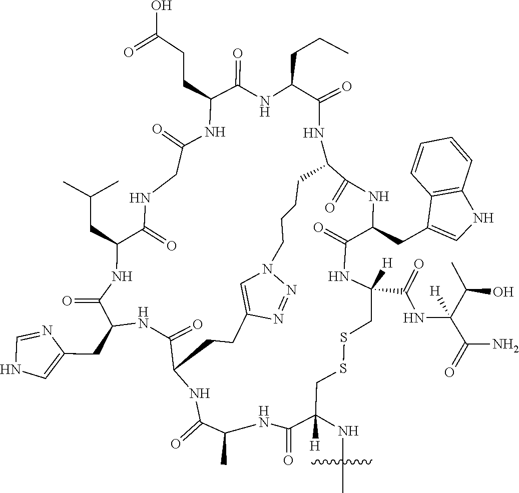 [SMILES: *N[C@H]1CSSC[C@@H](C(=O)N[C@H](C(N)=O)[C@@H](C)O)NC(=O)[C@H](Cc2c[nH]c3ccccc23)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CCC)C(=O)N2] | A-27 |
| 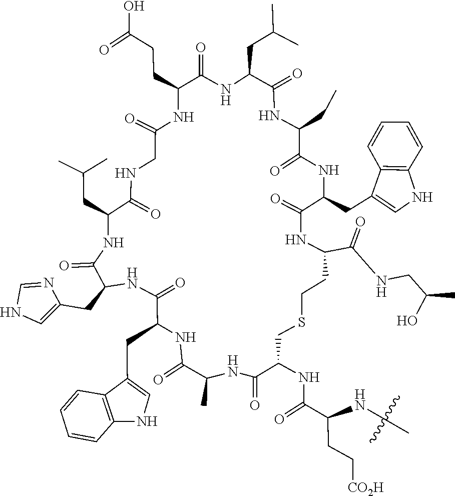 [SMILES: *N[C@@H](CCC(=O)O)C(=O)N[C@H]1CSCC[C@@H](C(=O)NC[C@@H](C)O)NC(=O)[C@H](Cc2c[nH]c3ccccc23)NC(=O)[C@H](CC)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-28 |
| 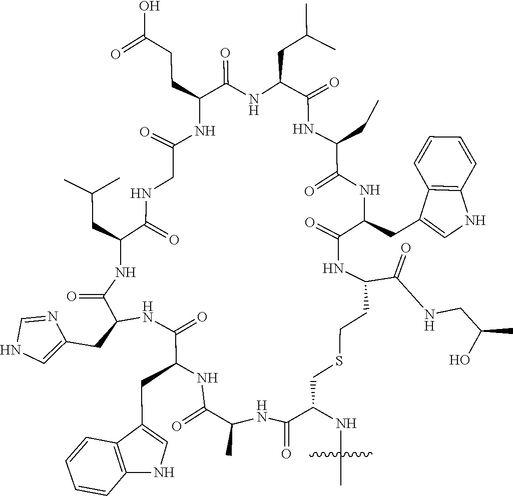 [SMILES: *N[C@H]1CSCC[C@@H](C(=O)NC[C@@H](C)O)NC(=O)[C@H](Cc2c[nH]c3ccccc23)NC(=O)[C@H](CC)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-29 |
| 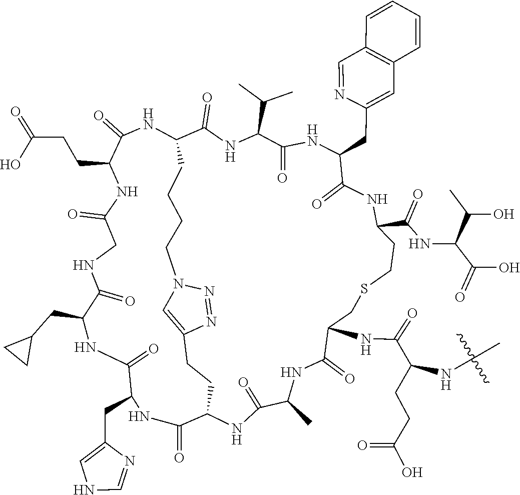 [SMILES: *N[C@@H](CCC(=O)O)C(=O)N[C@H]1CSCC[C@@H](C(=O)N[C@H](C(=O)O)C(C)O)NC(=O)[C@H](Cc2cc3ccccc3cn2)NC(=O)[C@H](C(C)C)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-30 |
| 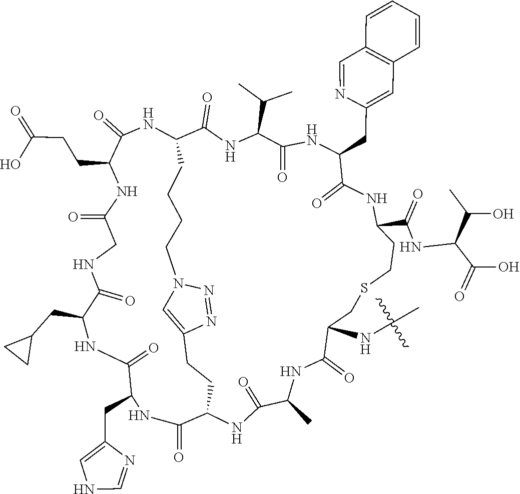 [SMILES: *N[C@H]1CSCC[C@@H](C(=O)N[C@H](C(=O)O)C(C)O)NC(=O)[C@H](Cc2cc3ccccc3cn2)NC(=O)[C@H](C(C)C)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-31 |
| 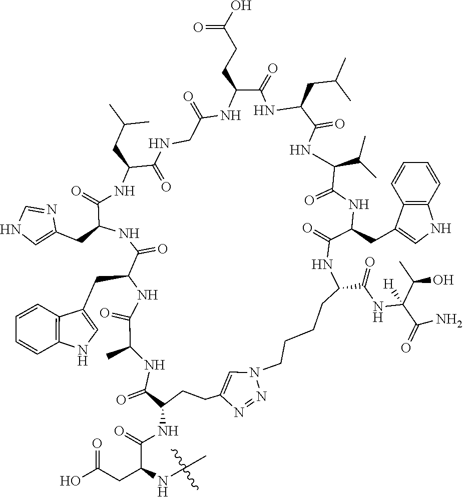 | A-32 |
| 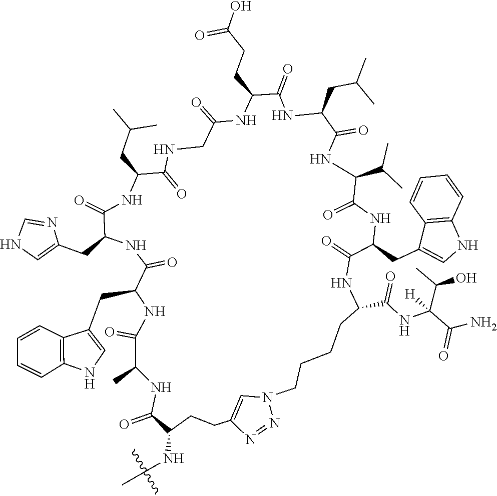 | A-33 |
| 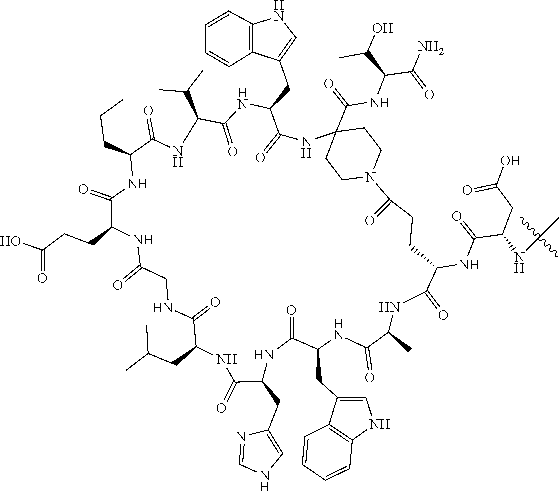 [SMILES: *N[C@@H](CC(=O)O)C(=O)N[C@H]1CCC(=O)N2CCC(C(=O)N[C@H](C(N)=O)C(C)O)(CC2)NC(=O)[C@H](Cc2c[nH]c3ccccc23)NC(=O)[C@H](C(C)C)NC(=O)[C@H](CCC)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-34 |
| 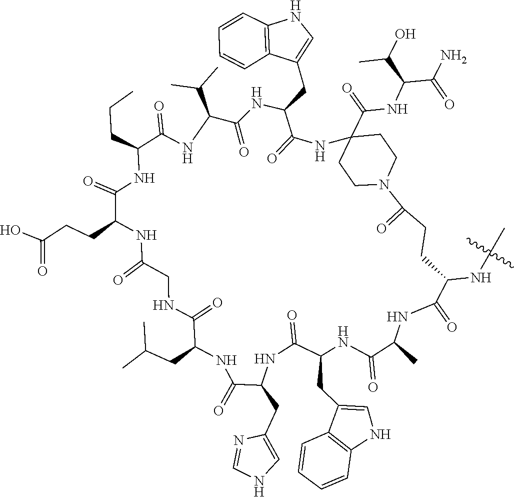 [SMILES: *N[C@H]1CCC(=O)N2CCC(C(=O)N[C@H](C(N)=O)C(C)O)(CC2)NC(=O)[C@H](Cc2c[nH]c3ccccc23)NC(=O)[C@H](C(C)C)NC(=O)[C@H](CCC)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-35 |
| 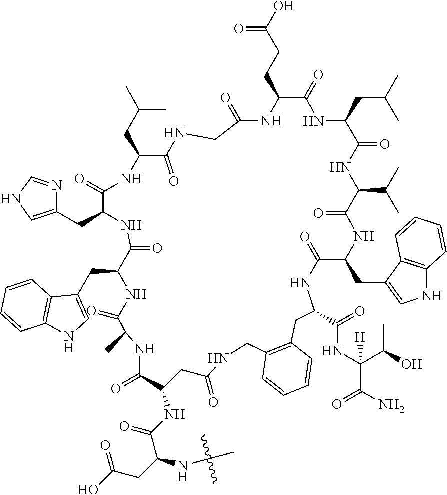 | A-36 |
| 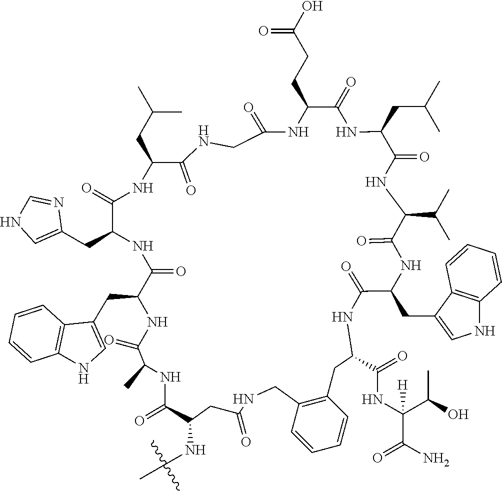 | A-37 |
| 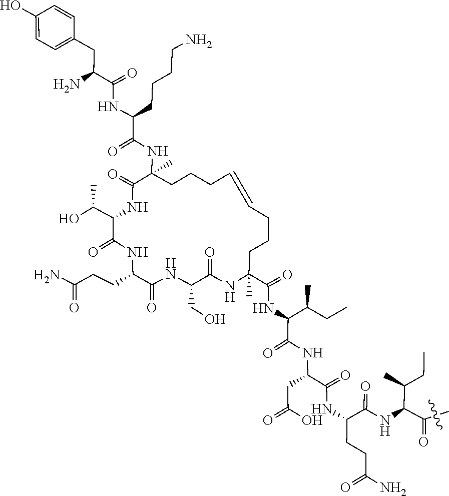 | A-38 |
| 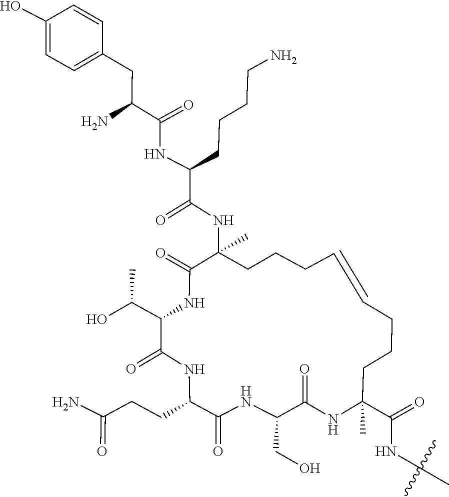 [SMILES: *NC(=O)[C@]1(C)CCC/C=C/CCC[C@](C)(NC(=O)[C@H](CCCCN)NC(=O)[C@@H](N)Cc2ccc(O)cc2)C(=O)N[C@@H]([C@@H](C)O)C(=O)N[C@@H](CCC(N)=O)C(=O)N[C@@H](CO)C(=O)N1] | A-39 |
| 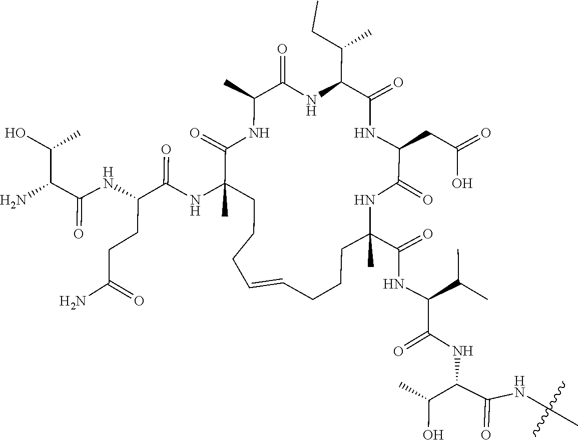 [SMILES: *NC(=O)[C@@H](NC(=O)[C@@H](NC(=O)[C@]1(C)CCC/C=C/CCC[C@](C)(NC(=O)[C@H](CCC(N)=O)NC(=O)[C@H](N)[C@@H](C)O)C(=O)N[C@@H](C)C(=O)N[C@@H]([C@@H](C)CC)C(=O)N[C@@H](CC(=O)O)C(=O)N1)C(C)C)[C@@H](C)O] | A-40 |
| 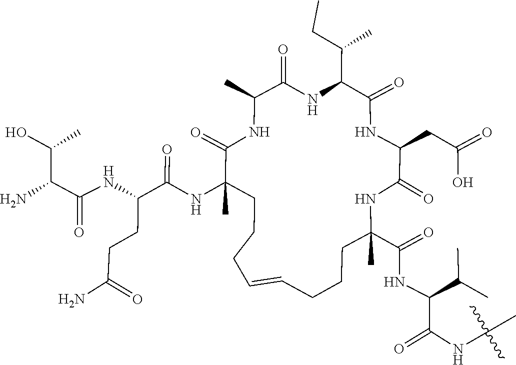 [SMILES: *NC(=O)[C@@H](NC(=O)[C@]1(C)CCC/C=C/CCC[C@](C)(NC(=O)[C@H](CCC(N)=O)NC(=O)[C@H](N)[C@@H](C)O)C(=O)N[C@@H](C)C(=O)N[C@@H]([C@@H](C)CC)C(=O)N[C@@H](CC(=O)O)C(=O)N1)C(C)C] | A-41 |
| 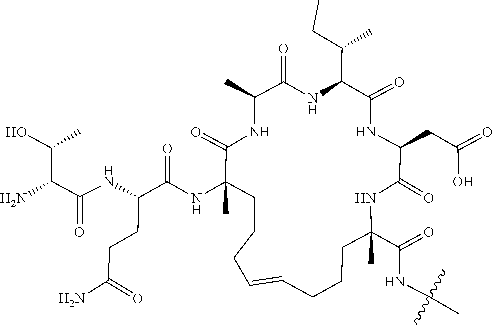 [SMILES: *NC(=O)[C@]1(C)CCC/C=C/CCC[C@](C)(NC(=O)[C@H](CCC(N)=O)NC(=O)[C@H](N)[C@@H](C)O)C(=O)N[C@@H](C)C(=O)N[C@@H]([C@@H](C)CC)C(=O)N[C@@H](CC(=O)O)C(=O)N1] | A-42 |
| 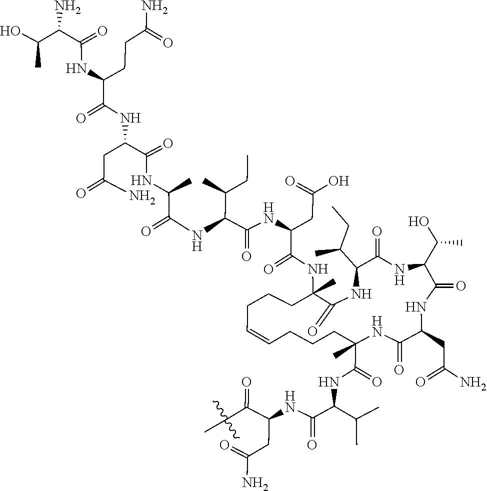 [SMILES: *C(=O)[C@H](CC(N)=O)NC(=O)[C@@H](NC(=O)[C@]1(C)CCC/C=C\CCC[C@](C)(NC(=O)[C@H](CC(=O)O)NC(=O)[C@@H](NC(=O)[C@H](C)NC(=O)[C@H](CC(=O)CN)NC(=O)[C@H](CCC(N)=O)NC(=O)[C@@H](N)[C@@H](C)O)[C@@H](C)CC)C(=O)N[C@@H]([C@@H](C)CC)C(=O)N[C@@H]([C@@H](C)O)C(=O)N[C@@H](CC(N)=O)C(=O)N1)C(C)C] | A-43 |
| 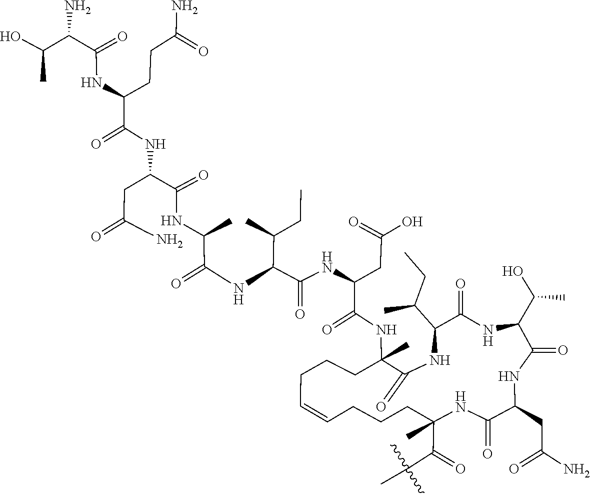 [SMILES: *C(=O)[C@]1(C)CCC/C=C\CCC[C@](C)(NC(=O)[C@H](CC(=O)O)NC(=O)[C@@H](NC(=O)[C@H](C)NC(=O)[C@H](CC(=O)CN)NC(=O)[C@H](CCC(N)=O)NC(=O)[C@@H](N)[C@@H](C)O)[C@@H](C)CC)C(=O)N[C@@H]([C@@H](C)CC)C(=O)N[C@@H]([C@@H](C)O)C(=O)N[C@@H](CC(N)=O)C(=O)N1] | A-44 |
| 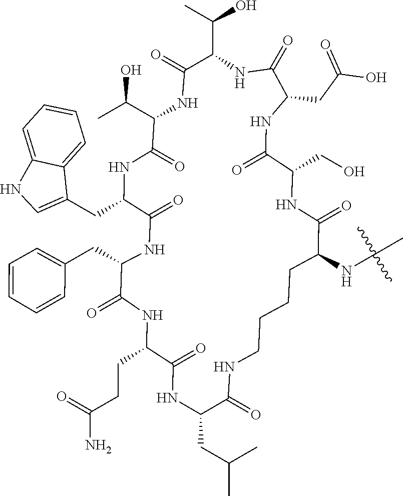 [SMILES: *N[C@H]1CCCCNC(=O)[C@H](CC(C)C)NC(=O)[C@H](CCC(N)=O)NC(=O)[C@H](Cc2ccccc2)NC(=O)[C@H](Cc2c[nH]c3ccccc23)NC(=O)[C@H]([C@@H](C)O)NC(=O)[C@H]([C@@H](C)O)NC(=O)[C@H](CC(=O)O)NC(=O)[C@H](CO)NC1=O] | A-45 |
| 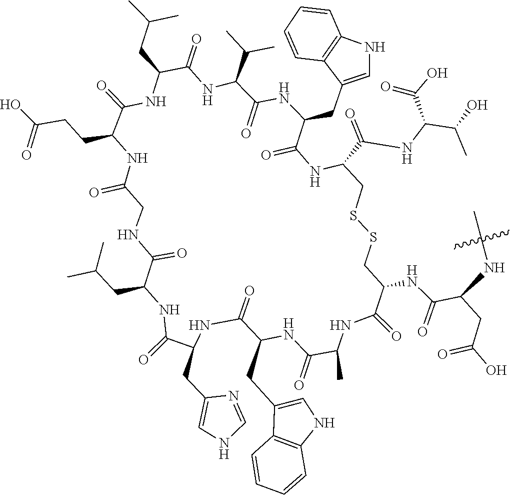 [SMILES: *N[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-46 |
| 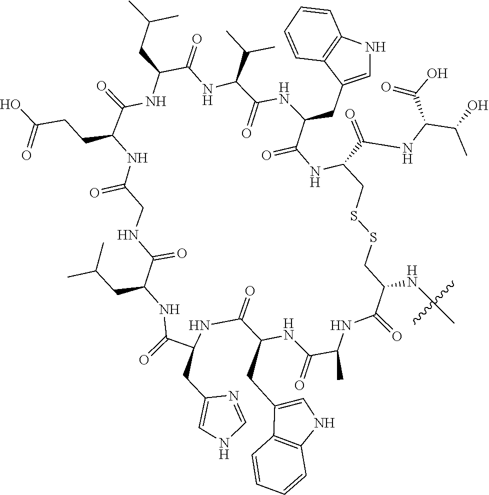 [SMILES: *N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-47 |
| 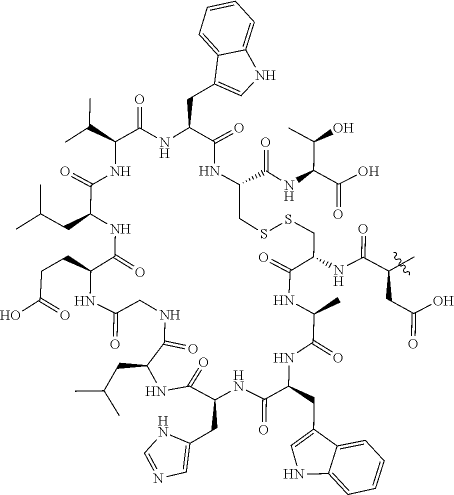 | A-48 |
| 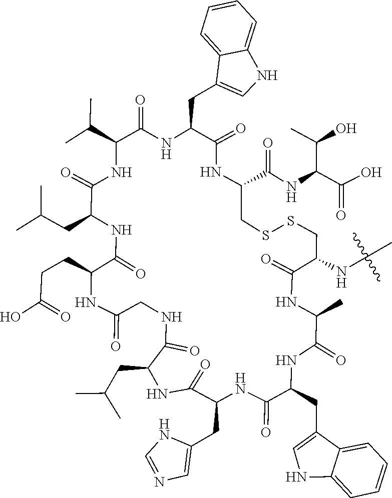 [SMILES: *N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2cnc[nH]2)NC(=O)[C@H](Cc2c[nH]c3ccccc23)NC(=O)[C@H](C)NC1=O] | A-49 |
| 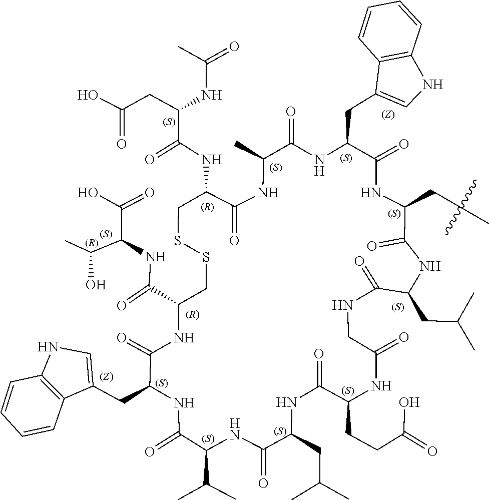 | A-50 |

In some embodiments, a target binding moiety is an antibody binding moiety described herein. In some embodiments, a protein binding moiety is an antibody binding moiety described herein. In some embodiments, —COOH and/or amino groups of amino acid residues, e.g., those at the C-terminus or N-terminus, is optionally capped. For example, in some embodiments, a —COOH group (e.g., a C-terminus —COOH) is amidated (e.g., converted into —CON(R′)₂, e.g., —C(O)NHR (e.g., —C(O)NH₂)), and in some embodiments, an amino group, e.g. —NH₂ (e.g., a N-terminus —NH₂) is capped with R′- or R′C(O)— (e.g., in some embodiments, by conversion —NH₂ into —NHR′ (e.g., —NHC(O)R, (e.g., —NHC(O)CH₃))).

In some embodiments, a target binding moiety is or comprises optionally substituted A-1, A-2, A-3, A-4, A-5, A-6, A-7, A-8, A-9, A-10, A-11, A-12, A-13, A-14, A-15, A-16, A-17, A-18, A-19, A-20, A-21, A-22, A-23, A-24, A-25, A-26, A-27, A-28, A-29, A-30, A-31, A-32, A-33, A-34, A-35, A-36, A-37, A-38, A-39, A-40, A-41, A-42, A-43, A-44, A-45, A-46, A-47, A-48, A-49, or A-50, each of which is optionally substituted. In some embodiments, such a target binding moiety is an antibody binding moiety. In some embodiments, such a target binding moiety is a universal antibody binding moiety.

In some embodiments, a target binding moiety, e.g., a protein binding moiety (e.g., an antibody binding moiety (e.g., a universal antibody binding moiety)) comprises a peptide unit, and is connected to a linker moiety through the C-terminus of the peptide unit. In some embodiments, it is connected to a linker moiety through the N-terminus of the peptide unit. In some embodiments, it is connected to a linker through a side chain group of the peptide unit. In some embodiments, an antibody binding moiety, e.g., a universal antibody binding moiety comprises a peptide unit, and is connected to a target binding moiety optionally through a linker moiety through the C-terminus of the peptide unit. In some embodiments, a target binding moiety, e.g., a protein binding moiety (e.g., an antibody binding moiety (e.g., a universal antibody binding moiety)) comprises a peptide unit, and is connected to a target binding moiety optionally through a linker moiety through the N-terminus of the peptide unit. In some embodiments, In some embodiments, a target binding moiety, e.g., a protein binding moiety (e.g., an antibody binding moiety (e.g., a universal antibody binding moiety)) comprises a peptide unit, and is connected to a target binding moiety optionally through a linker moiety through a side chain of the peptide unit.

In some embodiments, a target binding moiety is or comprises (DCAWHLGELVWCT)-, wherein 1-5 (e.g., 1, 2, 3, 4, or 5) amino acid residues may be independently and optionally replaced with another amino acid residue, 1-5 (e.g., 1, 2, 3, 4, or 5) amino acid residues may be independently and optionally deleted, and/or 1-5 (e.g., 1, 2, 3, 4, or 5) amino acid residues may be independently and optionally inserted. In some embodiments, it is connected to the rest of a molecule through its N-terminus. In some embodiments, it is connected to the rest of a molecule through its C-terminus. In some embodiments, it is connected to the rest of a molecule through a side chain of an amino acid residue (e.g., various X residues as described in the present disclosure). In some embodiments, two cysteine residues form a disulfide bond. In some embodiments, a target binding moiety is or comprises

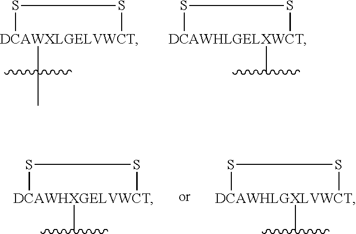

wherein X is an amino acid residue bonded to the rest of a compound or agent, and wherein 1-5 (e.g., 1, 2, 3, 4, or 5) amino acid residues may be independently and optionally replaced with another amino acid residue, 1-5 (e.g., 1, 2, 3, 4, or 5) amino acid residues may be independently and optionally deleted, and/or 1-5 (e.g., 1, 2, 3, 4, or 5) amino acid residues may be independently and optionally inserted. In some embodiments, the total number of replacements, deletions, and insertions is no more than 10 (e.g., 0, or no more than 1, 2, 3, 4, 5, 6, 7, 8, 9, or 10). In some embodiments, the total number is 0. In some embodiments, the total number is no more than 1. In some embodiments, the total number is no more than 2. In some embodiments, the total number is no more than 3. In some embodiments, the total number is no more than 4. In some embodiments, the total number is no more than 5. In some embodiments, the total number is no more than 6. In some embodiments, the total number is no more than 7. In some embodiments, the total number is no more than 8. In some embodiments, the total number is no more than 9. In some embodiments, the total number is no more than 10. In some embodiments, there are no insertions. In some embodiments, there are no deletions. In some embodiments, there are no replacements. In some embodiments X is X is an amino acid residue bonded to the rest of a compound or agent. In some embodiments, X is —N(R′)—CH(—)—C(O)—. In some embodiments, X is —N(R′)—CH(-L^{LG1}-)—C(O)—. In some embodiments, X is —N(R′)—CH(-L^{LG1}-L^{LG2}-)—C(O)—. In some embodiments, X is —N(R′)—CH(-L LG-L^{LG2}-L^{LG3}-. In some embodiments, X is —N(R′)—CH(-L^{LG1}-L^{LG2}-L^{LG3}-L^{LG4}-)—C(O)—.

In some embodiments, X is a residue of any of the following:

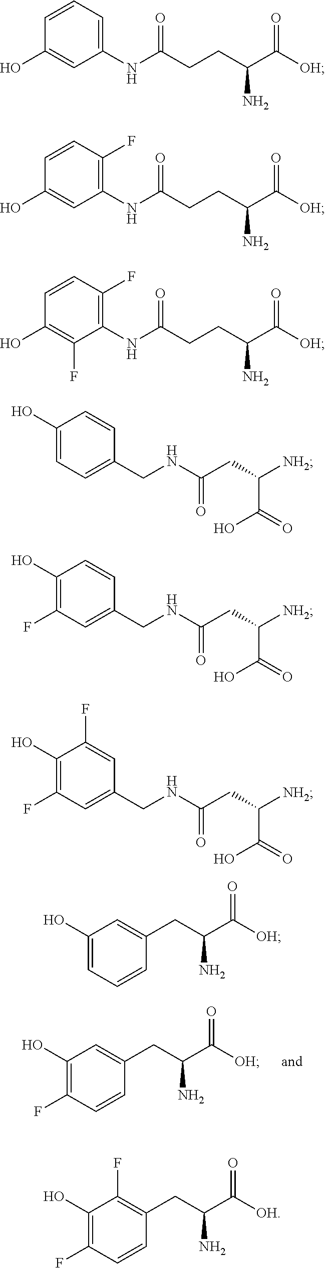

In some embodiments, X is K. In some embodiments, X is D. In some embodiments, X is a residue of Dab. In some embodiments, X is E.

In some embodiments, an antibody binding moiety, e.g., a universal antibody binding moiety, is or comprises a small molecule entity, with a molecular weight of, e.g., less than 10000, 9000, 8000, 7000, 6000, 5000, 4000, 3000, 2000, 1500, 1000, etc. Suitable such antibody binding moieties include small molecule Fc binder moieties, e.g., those described in U.S. Pat. No. 9,745,339, US 2013/0131321, etc. In some embodiments, an antibody binding moiety is of such a structure that its corresponding compound is a compound described in U.S. Pat. No. 9,745,339 or US 2013/0131321, the compounds of each of which are independently incorporated herein by reference. In some embodiments, an antibody binding moiety IG and HG is of such a structure that H-IG and H-HG is a compound described in U.S. Pat. No. 9,745,339 or US 2013/0131321, the compounds of each of which are independently incorporated herein by reference. In some embodiments, such a compound can bind to an antibody. In some embodiments, such a compound can bind to Fc region of an antibody.

In some embodiments, a target binding moiety is or comprises any of the following, each of which is optionally substituted:

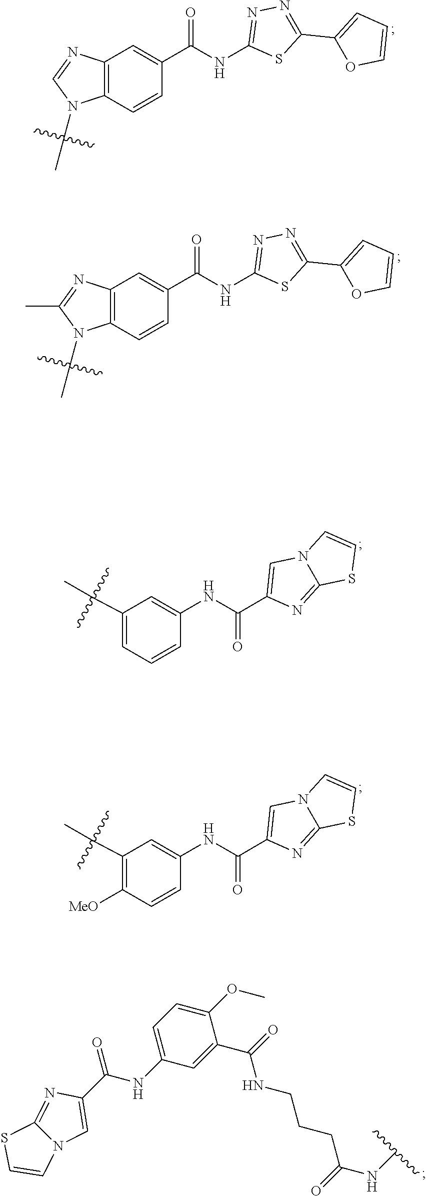 [SMILES: *n1cnc2cc(C(=O)Nc3nnc(-c4ccco4)s3)ccc21 ; *n1c(C)nc2cc(C(=O)Nc3nnc(-c4ccco4)s3)ccc21 ; *c1cccc(NC(=O)c2cn3ccsc3n2)c1 ; *c1cc(NC(=O)c2cn3ccsc3n2)ccc1OC ; *NC(=O)CCCNC(=O)c1cc(NC(=O)c2cn3ccsc3n2)ccc1OC]

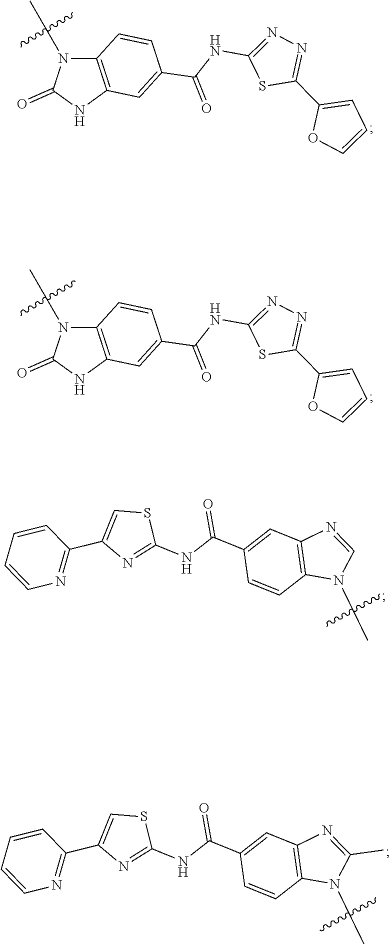 [SMILES: *n1c(=O)[nH]c2cc(C(=O)Nc3nnc(-c4ccco4)s3)ccc21 ; *n1c(=O)[nH]c2cc(C(=O)Nc3nnc(-c4ccco4)s3)ccc21 ; *n1cnc2cc(C(=O)Nc3nc(-c4ccccn4)cs3)ccc21 ; *n1c(C)nc2cc(C(=O)Nc3nc(-c4ccccn4)cs3)ccc21]

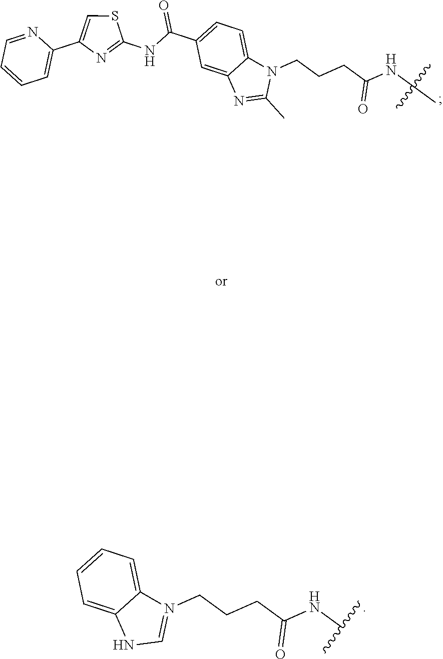

In some embodiments, target binding moiety is or comprises any of the following, each of which is optionally substituted:

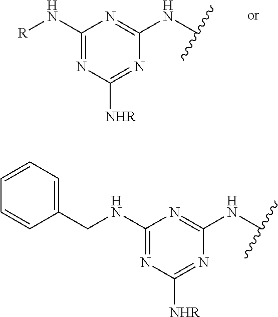

where R can be, for example, hydrogen, C₁-C₄alkyl, or C₃-C₆cycloalkyl.

In some embodiments, a target binding moiety is or comprises any of the following:

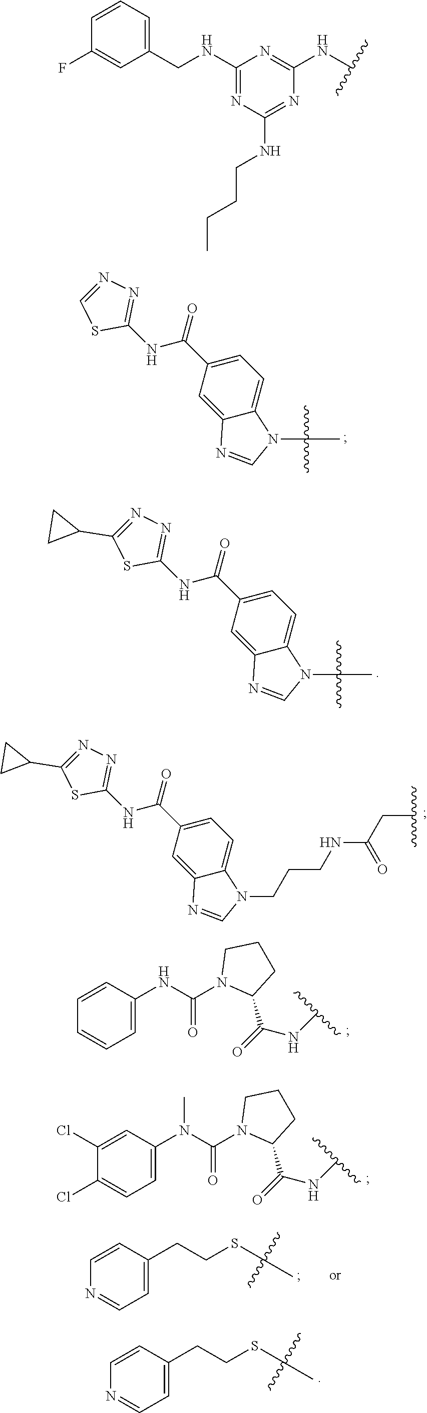

In some embodiments, target binding moiety is or comprises

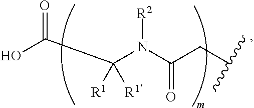

m wherein each variable is independently as described herein. In some embodiments, m is 4 to 13.

In some embodiments, a target binding moiety is or comprises

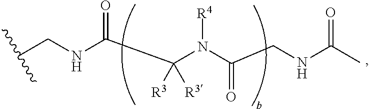

wherein b is 1-20, and each other variable is independently as described herein.

In some embodiments, b is 4-13. In some embodiments, a target binding moiety, e.g., R^{c}-(Xaa)z-, is or comprises any of the following:

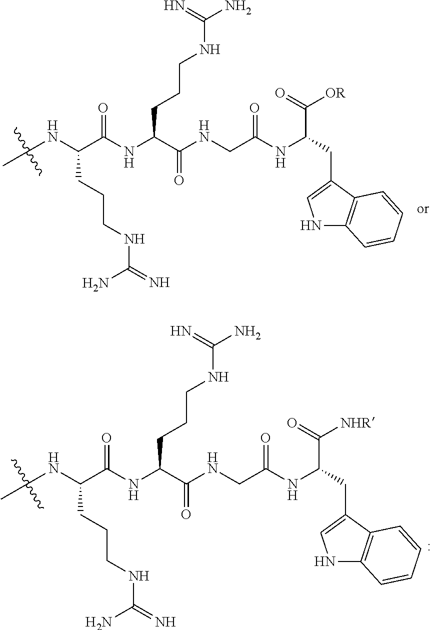

where R is, e.g., H or C₁-C₄ alkyl and R′ is e.g., H or C₁-C₄alkyl.

In some embodiments, a target binding moiety, e.g., R^{x}-(Xaa)z-, is or comprises

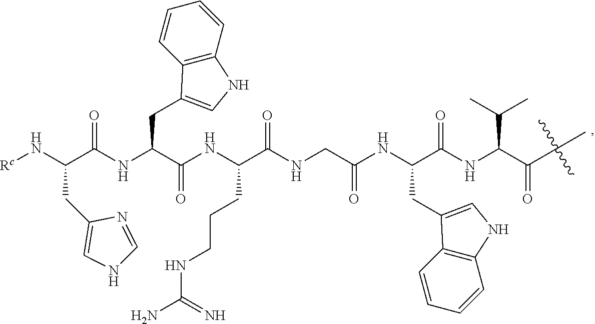

such as

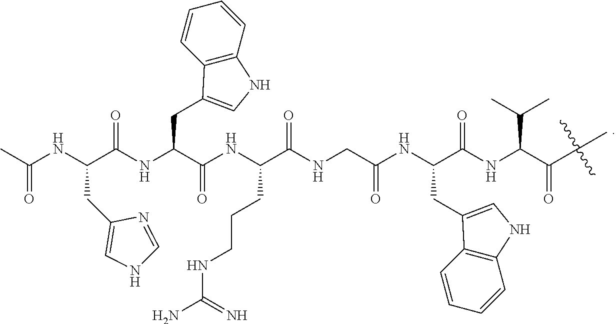 [SMILES: *C(=O)[C@@H](NC(=O)[C@H](Cc1c[nH]c2ccccc12)NC(=O)CNC(=O)[C@H](CCCNC(=N)N)NC(=O)[C@H](Cc1c[nH]c2ccccc12)NC(=O)[C@H](Cc1c[nH]cn1)NC(C)=O)C(C)C]

### I

n some embodiments, a target binding moiety e R^{c}-(Xaa)z-, is or comprises

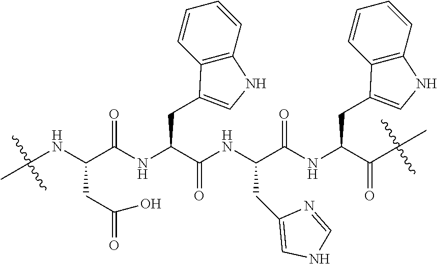 [SMILES: *N[C@@H](CC(=O)O)C(=O)N[C@@H](Cc1c[nH]c2ccccc12)C(=O)N[C@@H](Cc1c[nH]cn1)C(=O)N[C@@H](Cc1c[nH]c2ccccc12)C(*)=O]

such as

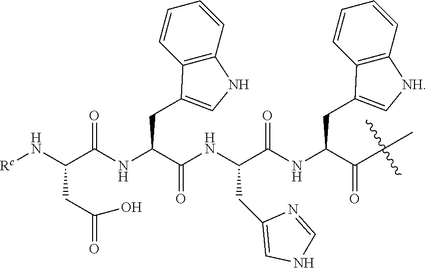

In some embodiments, a target binding moiety, e.g., R^{c}-(Xaa)z-, is or comprises

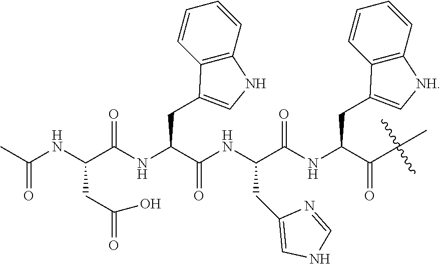 [SMILES: *C(=O)[C@H](Cc1c[nH]c2ccccc12)NC(=O)[C@H](Cc1c[nH]cn1)NC(=O)[C@H](Cc1c[nH]c2ccccc12)NC(=O)[C@H](CC(=O)O)NC(C)=O]

In some embodiments, a target binding moiety, e.g., R^{c}-(Xaa)z-, is or comprises any one of the following:

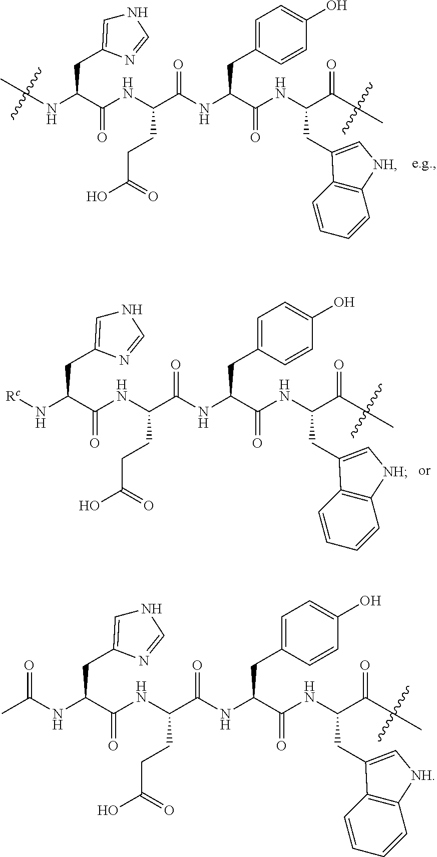

In some embodiments, a target binding moiety, e.g., R^{c}-(Xaa)z-, is or comprises

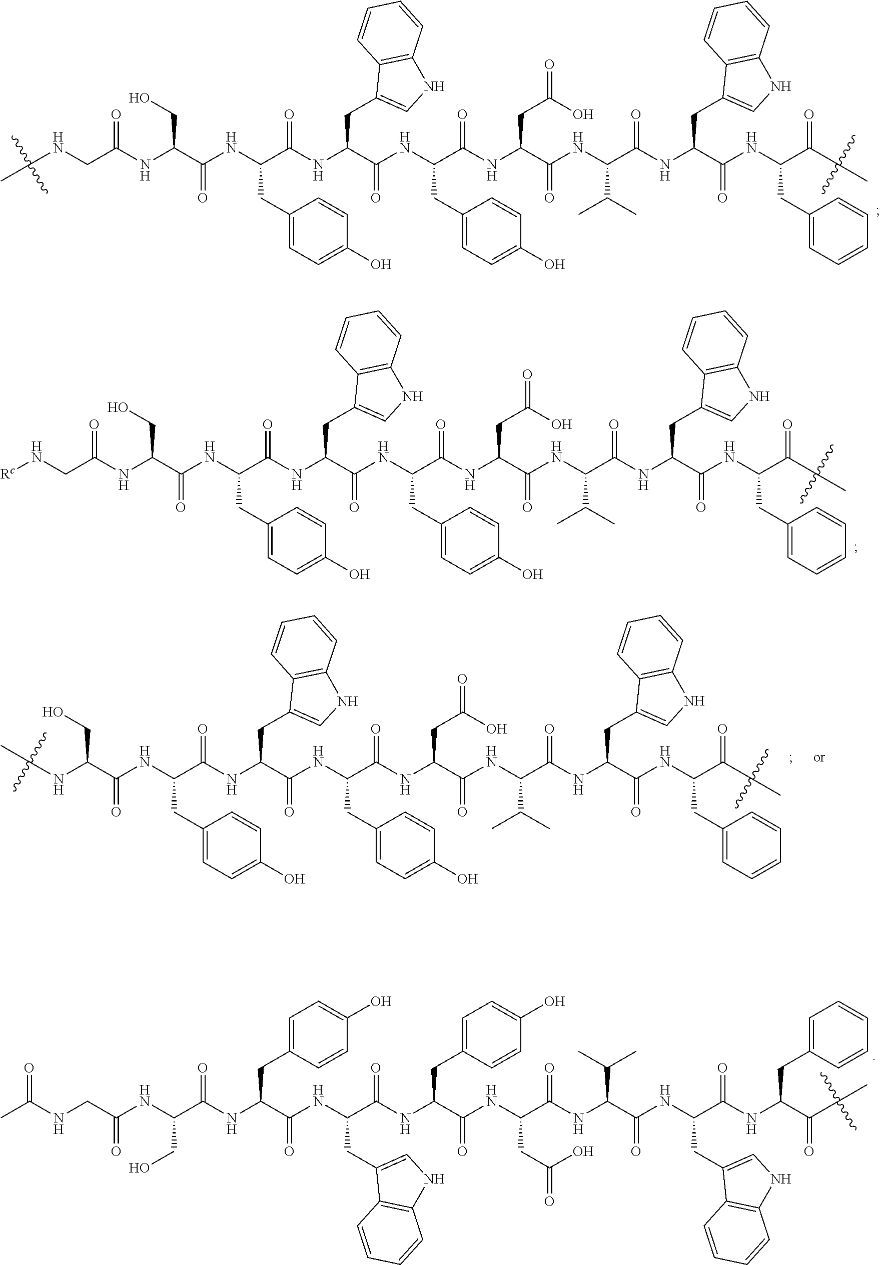

In some embodiments, a target binding moiety, e.g., R^{c}-(Xaa)z-, is or comprises

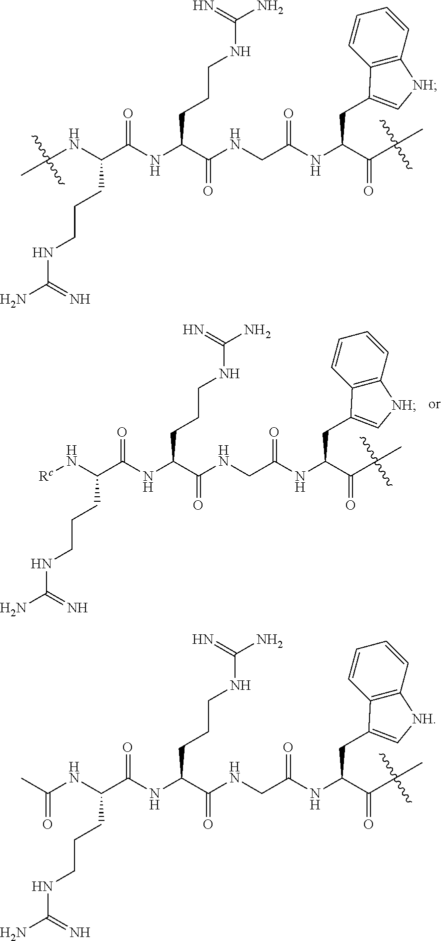

In some embodiments, —NH— is bonded to a R^{c} group. In some embodiments, R^{c} is R—C(O)—. In some embodiments, R^{c} is CH₃C(O)—. In some embodiments, such target binding moieties are antibody binding moieties.

In some embodiments, a target binding moiety, e.g.,

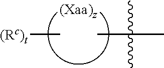

or R^{c}-(Xaa)z-, is or comprises any one of the following:

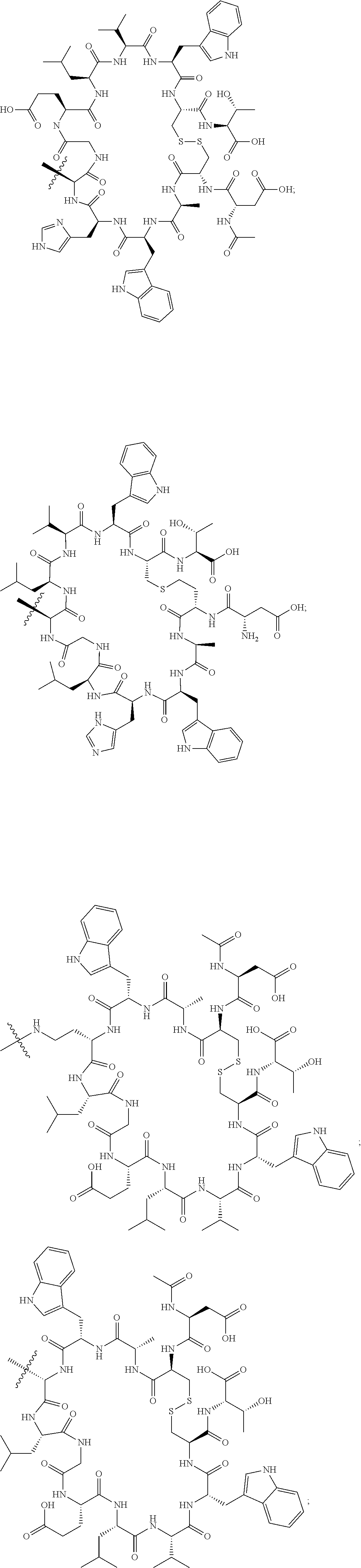

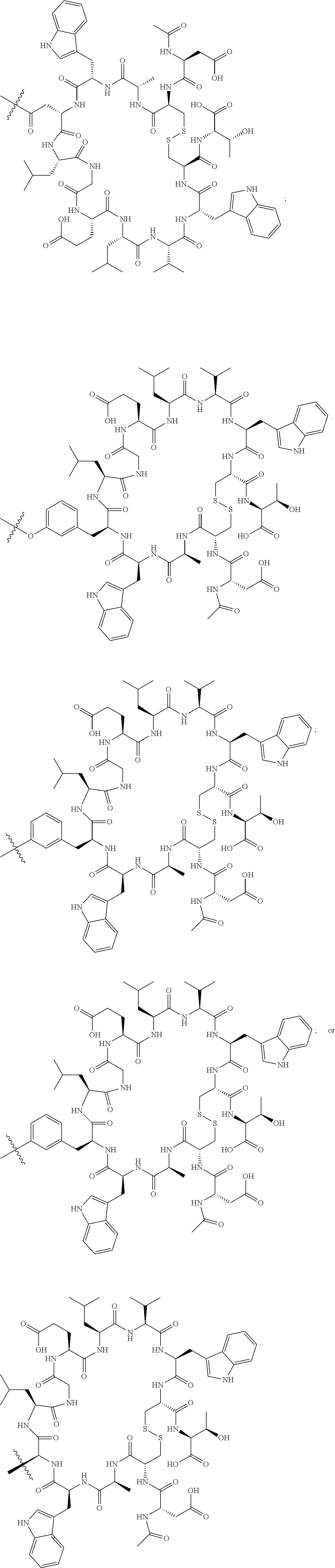

In some embodiments, a target binding moiety, e.g., R^{c}-(Xaa)z-, is or comprises a Z33 peptide moiety. In some embodiments, a target binding moiety, e.g., R^{c}-(Xaa)z-, is or comprises -FNMQQQRRFYEALHDPNLNEEQRNAKIKSIRDD-NH₂ or a fragment thereof. In some embodiments, a target binding moiety, e.g., R^{c}-(Xaa)z-, is or comprises FNMQCQRRFYEALHDPNLNEEQRNAKIKSIRDDC or a fragment thereof. In some embodiments, a target binding moiety, e.g.,

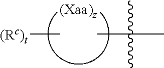

or R^{c}-(Xaa)z-, is or comprises a moiety of a peptide such as FNMQCQRRFYEALHDPNLNEEQRNAKIKSIRDDC, RGNCAYHRGQLVWCTYH, RGNCAYHKGQLVWCTYH, RGNCKYHRGQLVWCTYH, RGNCAWHRGKLVWCTYH, RGNCKWHRGELVWCTYH, RGNCKWHRGQLVWCTYH, RGNCKYHLGELVWCTYH, RGNCKYHLGQLVWCTYH, DCKWHLGELVWCT, DCKYHLGELVWCT, DCKWHRGELVWCT, DCKWHLGQLVWCT, DCKYHRGELVWCT, DCKYHLGQLVWCT, DCKWHRGQLVWCT, DCKYHRGQLVWCT, FNKQCQRRFYEALHDPNLNEEQRNARIRSIRDDC, FNMQCQRRFYEALHDPNLNEEQRNARIRSIKDDC, FNMQCQRRFYEALHDPNLNKEQRNARIRSIRDDC, FNMQCQRRFYEALHDPNLNEEQRNARIRSIRDDC, RGNCAWHLGQLVWCKYH, RGNCAWHLGELVWCKYH, RGNCAYHLGQLVWCTKH, RGNCAYHLGQLVWCTYK, RGNCAYHRGQLVWCTKH, KNMQCQRRFYEALHDPNLNEEQRNARIRSIRDDC, FNMQCQKRFYEALHDPNLNEEQRNARIRSIRDDC, FNMQCQRRFYEAKHDPNLNEEQRNARIRSIRDDC, FNMQCQRRFYEALHDPNLNEEQRKARIRSIRDDC, FNMQCQRRFYEALHDPNLNKEQRNARIRSIRDDC, FNMQCQRRFYEALHDPNLNEEQRNARIRSIKDDC, FNKQCQRRFYEALHDPNLNEEQRNARIRSIRDDC, FNMQCKRRFYEALHDPNLNEEQRNARIRSIRDDC, FNMQCQRRFYEALHDPNLNEEQRNARIRSIRKDC, Fc-III, FcBP-2, Fc-III-4C

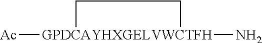

(X=K or R), etc., wherein two cysteine residues may optionally form a disulfide bond. In some embodiments, in a peptide described herein, two cysteine residues form a disulfide bond. In some embodiments, a peptide, such as Z33, FNMQCQRRFYEALHDPNLNEEQRNAKIKSIRDDC, RGNCAYHRGQLVWCTYH, RGNCKYHRGQLVWCTYH, RGNCAYHKGQLVWCTYH, RGNCAWHRGKLVWCTYH, RGNCKWHRGQLVWCTYH, RGNCKWHRGELVWCTYH, RGNCKYHLGELVWCTYH, RGNCKYHLGQLVWCTYH, DCKWHLGELVWCT, DCKYHLGELVWCT, DCKWHRGELVWCT, DCKWHLGQLVWCT, DCKYHRGELVWCT, DCKYHLGQLVWCT, DCKWHRGQLVWCT, DCKYHRGQLVWCT, FNKQCQRRFYEALHDPNLNEEQRNARIRSIRDDC, FNMQCQRRFYEALHDPNLNEEQRNARIRSIKDDC, FNMQCQRRFYEALHDPNLNKEQRNARIRSIRDDC, FNMQCQRRFYEALHDPNLNEEQRNARIRSIRDDC, RGNCAWHLGQLVWCKYH, RGNCAWHLGELVWCKYH, RGNCAYHLGQLVWCTKH, RGNCAYHLGQLVWCTYK, RGNCAYHRGQLVWCTKH, KNMQCQRRFYEALHDPNLNEEQRNARIRSIRDDC, FNMQCQKRFYEALHDPNLNEEQRNARIRSIRDDC, FNMQCQRRFYEAKHDPNLNEEQRNARIRSIRDDC, FNMQCQRRFYEALHDPNLNEEQRKARIRSIRDDC, FNMQCQRRFYEALHDPNLNKEQRNARIRSIRDDC, FNMQCQRRFYEALHDPNLNEEQRNARIRSIKDDC, FNKQCQRRFYEALHDPNLNEEQRNARIRSIRDDC, FNMQCKRRFYEALHDPNLNEEQRNARIRSIRDDC, FNMQCQRRFYEALHDPNLNEEQRNARIRSIRKDC, Fc-III, FcBP-2, Fc-III-4C,

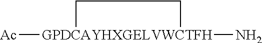

(X=K or R), etc., is connected through its N-terminus, C-terminus, or a side chain (e.g., of K (e.g., of underlined K residues in RGNCAYHKGQLVWCTYH, RGNCKYHRGQLVWCTYH, RGNCAWHRGKLVWCTYH, RGNCKWHRGELVWCTYH, RGNCKWHRGQLVWCTYH, RGNCKYHLGELVWCTYH, RGNCKYHLGQLVWCTYH, DCKWHLGELVWCT, DCKYHLGELVWCT, DCKWHRGELVWCT, DCKWHLGQLVWCT, DCKYHRGELVWCT, DCKYHLGQLVWCT, DCKWHRGQLVWCT, DCKYHRGQLVWCT, RGNCAWHLGQLVWCKYH, FNKQCQRRFYEALHDPNLNEEQRNARIRSIRDDC, FNMQCQRRFYEALHDPNLNEEQRNARIRSIKDDC, FNMQCQRRFYEALHDPNLNKEQRNARIRSIRDDC, RGNCAWHLGELVWCKYH, RGNCAYHLGQLVWCTKH, RGNCAYHLGQLVWCTYK, RGNCAYHRGQLVWCTKH, KNMQCQRRFYEALHDPNLNEEQRNARIRSIRDDC, FNMQCQKRFYEALHDPNLNEEQRNARIRSIRDDC, FNMQCQRRFYEAKHDPNLNEEQRNARIRSIRDDC, FNMQCQRRFYEALHDPNLNEEQRKARIRSIRDDC, FNMQCQRRFYEALHDPNLNKEQRNARIRSIRDDC, FNMQCQRRFYEALHDPNLNEEQRNARIRSIKDDC, FNKQCQRRFYEALHDPNLNEEQRNARIRSIRDDC, FNMQCKRRFYEALHDPNLNEEQRNARIRSIRDDC, FNMQCQRRFYEALHDPNLNEEQRNARIRSIRKDC, etc.)). In some embodiments, one or more amino acid residues of a sequence may be independently and optionally replaced (e.g., 1-5), deleted (e.g., 1-5) and/or inserted (e.g., 1-5) as described herein. In some embodiments, a target binding moiety, e.g.,

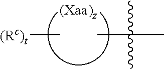

or R^{c}-(Xaa)z-, is or comprises -CXYHXXXLVWC-, -XCXYHXXXLVWC-, -CXYHXXXLVWCX-, —X₀₋₃CXYHXXXLVWCX₀₋₃-, -XCXYHXXXLVWCXXX-XXXCXYHXXXLVWCXXX-, wherein each X is independently an amino acid residue, and the two C residues optionally form a disulfide bond. In some embodiments, X⁸ (the X after H) is Orn. In some embodiments, X⁸ is Dab. In some embodiments, X⁸ is Lys(Ac). In some embodiments, X⁸ is Orn(Ac). In some embodiments, X⁸ is Dab(Ac). In some embodiments, X⁸ is Arg. In some embodiments, X⁸ is Nle. In some embodiments, X⁸ is Nva. In some embodiments, X⁸ is Val. In some embodiments, X⁸ is Tle. In some embodiments, X⁸ is Leu. In some embodiments, X⁸ is Ala(tBu). In some embodiments, X⁸ is Cha. In some embodiments, X⁸ is Phe. In some embodiments, a target binding moiety, e.g.,

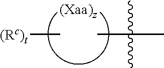

or R^{c}-(Xaa)z-, is or comprises DCAWHLGELVWCT. In some embodiments, a C-terminus and/or a N-terminus of a protein agent/peptide agent moiety are independently capped (e.g., RC(O)—such as CH₃C(O)— for N-terminus, —N(R′)₂ such as —NH₂ for C-terminus, etc.). In some embodiments, such target binding moieties are antibody binding moieties. In some embodiments, as described herein, a residue may be modified or replaced for connection with another moiety, e.g., in some embodiments, H may be replaced with an amino acid residue comprises a side chain that contain —COOH or a salt or activated form thereof (e.g., D).

In some embodiments, a target binding moiety, e.g.,

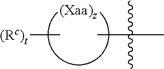

or R^{c}-(Xaa)z-, is or comprises (X₁₋₃)—C—(X₂)—H-(Xaa1)-G-(Xaa2)-L-V—W—C—(X₁₋₃), wherein each of X and Xaa is independently an amino acid residue and optionally not a cysteine residue. In some embodiments, Xaa1 is R, L, L, D, E, a 2-amino suberic acid residue, or a diaminopropionic acid residue. In some embodiments, Xaa2 is L, D, E, N, or Q. In some embodiments, Xaa1 is a lysine residue, a cysteine residue, an aspartic acid residue, a glutamic acid residue, a 2-amino suberic acid residue, or a diaminopropionic acid residue. In some embodiments, Xaa2 is a glutamic acid residue or an aspartic acid residue. In some embodiments, Xaa1 is an arginine residue or a leucine residue. In some embodiments, Xaa2 is a lysine residue, a glutamine residue, or an aspartic acid residue. In some embodiments, such target binding moieties are antibody binding moieties.

In some embodiments, a target binding moiety, e.g.,

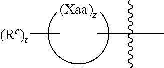

or R^{c}-(Xaa)z-, is or comprises (X¹-3)-C-(Xaa3)-(xaa4)-H-(Xaa1)-G-(Xaa2)-L-V-W-C-(Xaa5)-(Xaa6)-(Xaa7), wherein each of X and Xaa is independently an amino acid residue and optionally not a cysteine residue. In some embodiments, Xaa3 is an alanine residue or a lysine residue. In some embodiments, Xaa4 is a tryptophan residue or a tyrosine residue. In some embodiments, Xaa1 is an arginine residue, a leucine residue, a lysine residue, an aspartic acid residue, a glutamic acid residue, a 2-amino suberic acid residue, or a diaminopropionic acid residue. In some embodiments, Xaa2 is a lysine residue, a glutamine residue, a glutamic acid residue, an asparagine residue, or an aspartic acid residue. In some embodiments, Xaa5 is a threonine residue or a lysine residue. In some embodiments, Xaa6 is a tyrosine residue, a lysine residue, or absent. In some embodiments, Xaa7 is a histidine residue, a lysine residue, or absent. In some embodiments, such target binding moieties are antibody binding moieties.

In some embodiments, a target binding moiety, e.g.,

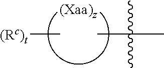

or R^{c}-(Xaa)z-, is or comprises D-C-(Xaa3)-(Xaa4)-H-(Xaa1)-G-(Xaa2)-L-V—W—C-(Xaa5)-(Xaa6)-(Xaa7), wherein each of X and Xaa is independently an amino acid residue and optionally not a cysteine residue. In some embodiments, Xaa3 is an alanine residue or a lysine residue. In some embodiments, Xaa4 is a tryptophan residue or a tyrosine residue. In some embodiments, Xaa1 is an arginine residue, a leucine residue, a lysine residue, an aspartic acid residue, a glutamic acid residue, a 2-amino suberic acid residue, or a diaminopropionic acid residue. In some embodiments, Xaa2 is a lysine residue, a glutamine residue, a glutamic acid residue, an asparagine residue, or an aspartic acid residue. In some embodiments, Xaa5 is a threonine residue or a lysine residue. In some embodiments, Xaa6 is a tyrosine residue, a lysine residue, or absent. In some embodiments, Xaa7 is a histidine residue, a lysine residue, or absent. In some embodiments, such target binding moieties are antibody binding moieties.

In some embodiments, a target binding moiety, e.g.,

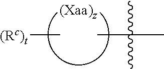

or R^{c}-(Xaa)z-, is or comprises D-C-(Xaa3)-(Xaa4)-H-(Xaa1)-G-(Xaa2)-L-V-W-C-T, wherein each of X and Xaa is independently an amino acid residue and optionally not a cysteine residue. In some embodiments, Xaa3 is an alanine residue or a lysine residue. In some embodiments, Xaa4 is a tryptophan residue or a tyrosine residue. In some embodiments, Xaa1 is an arginine residue, a leucine residue, a lysine residue, an aspartic acid residue, a glutamic acid residue, a 2-amino suberic acid residue, or a diaminopropionic acid residue. In some embodiments, Xaa2 is a lysine residue, a glutamine residue, a glutamic acid residue, an asparagine residue, or an aspartic acid residue. In some embodiments, such target binding moieties are antibody binding moieties.

In some embodiments, a target binding moiety, e.g.,

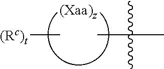

or R^{c}-(Xaa)z-, is or comprises R-G-N—C-(Xaa3)-(Xaa4)-H-(Xaa1)-G-(Xaa2)-L-V-W-C-(Xaa5)-(Xaa6)-(Xaa7), wherein each of X and Xaa is independently an amino acid residue and optionally not a cysteine residue. In some embodiments, Xaa3 is an alanine residue or a lysine residue. In some embodiments, Xaa4 is a tryptophan residue or a tyrosine residue. In some embodiments, Xaa1 is an arginine residue, a leucine residue, a lysine residue, an aspartic acid residue, a glutamic acid residue, a 2-amino suberic acid residue, or a diaminopropionic acid residue. In some embodiments, Xaa2 is a lysine residue, a glutamine residue, a glutamic acid residue, an asparagine residue, or an aspartic acid residue. In some embodiments, Xaa5 is a threonine residue or a lysine residue. In some embodiments, Xaa6 is a tyrosine residue, a lysine residue, or absent. In some embodiments, Xaa7 is a histidine residue, a lysine residue, or absent. In some embodiments, such target binding moieties are antibody binding moieties.

In some embodiments, target binding moieties, e.g., various target binding moieties described above, are protein binding moieties. In some embodiments, target binding moieties are antibody binding moieties. In some embodiments, LG is or comprises such a target binding moiety. In some embodiments, LG is or comprises a protein binding moiety. In some embodiments, LG is or comprises an antibody binding moiety.

In some embodiments, a target binding moiety, e.g., an antibody binding moiety, is or comprises an adapter protein agent, e.g., as described in Hui et al. Bioconjugate Chem. 2015, 26, 1456-1460. In some embodiments, when utilized in accordance with the present disclosure, adapter proteins do not require reactive residues (e.g., BPA) to achieve one or more or all advantages.

In some embodiments, target binding moiety, e.g., an antibody binding moiety is or comprises a triazine moiety, e.g., one is described in US 2009/0286693. In some embodiments, a target binding moiety, e.g., an antibody binding moiety is of such a structure that its corresponding compound is a compound described in US 2009/0286693, the compounds of which are independently incorporated herein by reference. In some embodiments, a target binding moiety, e.g., an antibody binding moiety, is ABT. In some embodiments, ABT is of such a structure that H-ABT is a compound described in US 2009/0286693, the compounds of which are independently incorporated herein by reference. In some embodiments, such a compound can bind to an antibody. In some embodiments, such a compound can bind to Fc region of an antibody.

In some embodiments, a target binding moiety, e.g., an antibody binding moiety is or comprises a triazine moiety, e.g., one described in Teng et al. “A strategy for the generation of biomimetic ligands for affinity chromatography. Combinatorial synthesis and biological evaluation of an IgG binding ligand” J. Mol. Recognit. 1999; 12: 67-75. In some embodiments, a target binding moiety, e.g., an antibody binding moiety is of such a structure that its corresponding compound is a compound described in Teng, the compounds of which are independently incorporated herein by reference. In some embodiments, a target binding moiety, e.g., an antibody binding moiety, ABT is of such a structure that H-ABT is a compound described in Teng, the compounds of which are independently incorporated herein by reference. In some embodiments, such a compound can bind to an antibody. In some embodiments, such a compound can bind to Fc region of an antibody.

In some embodiments, a target binding moiety, e.g., an antibody binding moiety is a triazine moiety, e.g., one described in Uttamchandani et al. “Microarrays of Tagged Combinatorial Triazine Libraries in the Discovery of Small-Molecule Ligands of Human IgG” J Comb Chem. 2004, 6(6): 862-8. In some embodiments, a target binding moiety, e.g., an antibody binding moiety is of such a structure that its corresponding compound is a compound described in Uttamchandani, the compounds of which are independently incorporated herein by reference. In some embodiments, a target binding moiety, e.g., an antibody binding moiety, ABT is of such a structure that H-ABT is a compound described in Uttamchandani, the compounds of which are independently incorporated herein by reference. In some embodiments, such a compound can bind to an antibody. In some embodiments, such a compound can bind to Fc region of an antibody.

In some embodiments, an antibody binding moiety binds to one or more binding sites of protein A. In some embodiments, an antibody binding moiety binds to one or more binding sites of protein G. In some embodiments, an antibody binding moiety binds to one or more binding sites of protein L. In some embodiments, an antibody binding moiety binds to one or more binding sites of protein Z. In some embodiments, an antibody binding moiety binds to one or more binding sites of protein LG. In some embodiments, an antibody binding moiety binds to one or more binding sites of protein LA. In some embodiments, an antibody binding moiety binds to one or more binding sites of protein AG.

In some embodiments, a target binding moiety, e.g., an antibody binding moiety can bind to a nucleotide-binding site. In some embodiments, a target binding moiety, e.g., an antibody binding moiety is a small molecule moiety that can bind to a nucleotide-binding site. In some embodiments, a small molecule is tryptamine. In some embodiments, a target binding moiety, e.g., an antibody binding moiety, ABT is of such a structure that H-ABT is tryptamine.

In some embodiments, an antibody binding moiety is a moiety (e.g., small molecule moiety, peptide moiety, nucleic acid moiety, etc.) that can selectively bind to IgG, and when used in provided technologies can provide and/or stimulate ADCC and/or ADCP. In some embodiments, peptide display technologies (e.g., phase display, non-cellular display, etc.) can be utilized to identify antibody binding moieties. In some embodiments, an antibody binding moiety is a moiety (e.g., small molecule moiety, peptide moiety, nucleic acid moiety, etc.) that can bind to IgG and optionally can compete with known antibody binders, e.g., protein A, protein G, protein L, etc.

As appreciated by those skilled in the art, antibodies of various properties and activities (e.g., antibodies recognizing different antigens, having optional modifications, etc.) may be targeted by antibody binding moieties described in the present disclosure. In some embodiments, such antibodies include antibodies administered to a subject, e.g., for therapeutic purposes. In some embodiments, antibody binding moieties described herein may bind antibodies toward different antigens and are useful for conjugating moieties of interest with various antibodies.

In some embodiments, a target binding moiety, e.g., an antibody binding moiety, is or comprises a meditope agent moiety. In some embodiments, a meditope agent is described in, e.g., US 2019/0111149.

In some embodiments, a target binding moiety, e.g., an antibody binding moiety, can bind to human IgG. In some embodiments, a target binding moiety, e.g., an antibody binding moiety, can bind to rabbit IgG. In some embodiments, a target binding moiety, e.g., an antibody binding moiety, binds to IgG1. In some embodiments, a target binding moiety, e.g., an antibody binding moiety, binds to IgG2. In some embodiments, a target binding moiety, e.g., an antibody binding moiety, binds to IgG3. In some embodiments, a target binding moiety, e.g., an antibody binding moiety, binds to IgG4. In some embodiments, a target binding moiety, e.g., an antibody binding moiety, binds to IgG1, IgG2 and/or IgG4. In some embodiments, a target binding moiety, e.g., an antibody binding moiety, binds to IgG1, IgG2 and IgG4.

In some embodiments, target binding moieties (e.g., antibody binding moieties) bind to targets (e.g., antibody agents for antibody binding moieties) with a Kd that is about 1 mM-1 pM or less. In some embodiments, a Kd is about 1 mM, 0.5 mM, 0.2 mM, 0.1 mM, 0.05 mM, 0.02 mM, 0.01 mM, 0.005 mM, 0.002 mM, 0.001 m M, 500 nM, 200 nM, 100 nM, 50 nM, 20 nM, 10 nM, 5 n M, 2 nM, 1 nM, 0.5 nM, 0.2 nM, 0.1 nM, or less. In some embodiments, Kd is about 1 mM or less. In some embodiments, Kd is about 0.5 mM or less. In some embodiments, Kd is about 0.1 mM or less. In some embodiments, Kd is about 0.05 mM or less. In some embodiments, Kd is about 0.01 mM or less. In some embodiments, Kd is about 0.005 mM or less. In some embodiments, Kd is about 0.001 mM or less. In some embodiments, Kd is about 500 nM or less. In some embodiments, Kd is about 200 nM or less. In some embodiments, Kd is about 100 nM or less. In some embodiments, Kd is about 50 nM or less. In some embodiments, Kd is about 20 nM or less. In some embodiments, Kd is about 10 nM or less. In some embodiments, Kd is about 5 nM or less. In some embodiments, Kd is about 2 nM or less. In some embodiments, Kd is about 1 nM or less. For example, in some embodiments, antibody binding moieties bind to IgG antibody agents with Kd described herein.

### Reactive Group

In some embodiments, provided compounds, e.g., those useful as reaction partners, comprise reactive groups (e.g., RG). As exemplified herein, in many embodiments, in provided compounds reactive groups (e.g., RG) are located between first groups (e.g., LG) and moieties of interest (e.g., MOI), and are optionally and independently linked to first groups and moieties of interest via linkers. In some embodiments, RG is a reaction group as described herein.

In some embodiments, as demonstrated herein, reactive groups when utilized in compounds that comprise no target binding moieties react slowly and provide low level of, in some embodiments, substantially no conjugation of moieties of interest with target agents. As demonstrated herein, combination of reactive groups with target binding moieties in the same compounds, e.g., as in compounds of formula R—I or salts thereof, can, among other things, promote reactions between reactive groups and target agents, enhance reaction efficiency, reduce side reactions, and/or improve reaction selectivity (e.g., in terms of target sites wherein conjugation of moieties of interest with target agents occurs).

Reactive groups in provided compounds can react with various types of groups in target agents. In some embodiments, reactive groups in provided compounds selectively react with amino groups of target agents, e.g., —NH₂ groups on side chains of lysine residues of proteins. In some embodiments, reactive groups when utilized in provided compounds, e.g., those of formula R—I or salts thereof, selectively react with particular sites of target agents, e.g., as shown in examples herein, one or more of K246, K248, K288, K290, K317, etc. of IgG1, K251, K 253, etc. for IgG2, K239, K241 for IgG4, etc. In some embodiments, a site is K246 or K248 of an antibody heavy chain. In some embodiments, sites are K246 and/or K248 of an antibody heavy chain. In some embodiments, a site is K246 of an antibody heavy chain. In some embodiments, a site is K248 of an antibody heavy chain. In some embodiments, a site is K288 or K290 of an antibody heavy chain. In some embodiments, a site is K288 of an antibody heavy chain. In some embodiments, a site is K290 of an antibody heavy chain. In some embodiments, a site is K317. In some embodiments, a site is K414 of an antibody heavy chain. In some embodiments, a site is K185 of an antibody light chain. In some embodiments, a site is K187 of an antibody light chain. In some embodiments, sites are K251 and/or K253 of an IgG2 heavy chain. In some embodiments, a site is K251 of an IgG2 heavy chain. In some embodiments, a site is K253 of an IgG2 heavy chain. In some embodiments, sites are K239 and/or K241 of an IgG4 heavy chain. In some embodiments, a site is K239 of an IgG4 heavy chain. In some embodiments, a site is K241 of an IgG4 heavy chain. In some embodiments, conjugation selectively occurs at one or more heavy chain sites over light chain sites. In some embodiments, for technologies without target binding moieties, conjugation occurs at light chain sites more than heavy chain sites (e.g., see FIG. 15).

In some embodiments, a reactive group, e.g., RG, is or comprises an ester group. In some embodiments, a reactive group, e.g., RG, is or comprises an electrophilic group, e.g., a Michael acceptor.

In some embodiments, a reactive group, e.g., RG, is or comprises -L^{RG1}-L^{RG2}-, wherein each of L^{RG1} and L^{RG2} is independently L as described herein. In some embodiments, a reactive group, e.g., RG, is or comprises -L^{LG4}-L^{RG1}-L^{RG2}-, wherein each variable is as described herein. In some embodiments, a reactive group, e.g., RG, is or comprises -L^{LG3}-L^{LG4}-L^{RG1}-L^{RG2}-, wherein each variable is as described herein. In some embodiments, a reactive group, e.g., RG, is or comprises -L^{LG2}-L^{LG3}-L^{LG4}-L^{RG1}-L^{RG2}-, wherein each variable is as described herein. In some embodiments, a reactive group, e.g., RG, is or comprises -L^{LG4}-L^{RG2}-, wherein each variable is as described herein. In some embodiments, a reactive group, e.g., RG, is or comprises -L^{LG3}-L^{LG4}-L^{RG2}-, wherein each variable is as described herein. In some embodiments, a reactive group, e.g., RG, is or comprises -L^{LG2}-L^{LG3}-L^{LG4}-L^{RG2}-, wherein each variable is as described herein.

In some embodiments, as described herein, L^{LG4} is —O—. In some embodiments, L^{LG4} is —N(R)—. In some embodiments, L^{LG4} is —NH—.

In some embodiments, as described herein, L^{LG3} is or comprises an optionally substituted aryl ring. In some embodiments, L^{LG3} is or comprises a phenyl ring. In some embodiments, an aryl or phenyl ring is substituted. In some embodiments, a substituent is an electron-withdrawing group as described herein, e.g., —NO₂, —F, etc.

In some embodiments, L^{RG1} is a covalent bond. In some embodiments, L^{RG1} is not a covalent bond. In some embodiments, L^{RG1} is —S(O)₂—.

In some embodiments, L^{RG2} is —C(O)—. In some embodiments, a reactive group is or comprises -L^{LG4}-C(O)—, wherein each variable is as described herein. In some embodiments, a reactive group is or comprises -L^{LG3}-L^{LG4}-C(O)—, wherein each variable is as described herein. In some embodiments, a reactive group is or comprises -L^{LG2}-L^{LG3}-L^{LG4}-C(O)—, wherein each variable is as described herein.

In some embodiments, L^{RG2} is -L^{RG3}-C(═CR^{RG1}R^{RG2})—CR^{RG3}R^{RG4}—, wherein each of R^{RG1}, R^{RG2}, R^{RG3} and R^{RG4} is independently -L-R′, and L^{RG3} is —C(O)—, —C(O)O—, —C(O)N(R′)—, —S(O)—, —S(O)₂—, —P(O)(OR′)—, —P(O)(SR′)—, or —P(O)(N(R′)₂)—. In some embodiments, each of R^{RG1}, R^{RG2}, R^{RG3} and R^{RG4} is independently R′. In some embodiments, one or more of R^{RG1}, R^{RG2}, R^{RG3} and R^{RG4} is independently —H. In some embodiments, L^{RG3} is —C(O)—. In some embodiments, L^{RG3} is —C(O)O—. In some embodiments, —O—, —N(R′)—, etc. of L^{RG3} is bonded to L^{PM}.

In some embodiments, R^{RG1} is —H. In some embodiments, R^{RG3} is —H.

In some embodiments, L^{RG2} is optionally substituted -L^{RG3}-C(═CHR^{RG2})—CHR^{RG4}—, wherein each variable is as described herein.

In some embodiments, R^{RG2} and R^{RG4} are taken together with their intervening atoms to form an optionally substituted ring as described herein. In some embodiments, a formed ring is an optionally substituted 3-10 membered monocyclic or bicyclic ring having 0-5 heteroatoms. In some embodiments, a formed ring is an optionally substituted 3-10 membered cycloaliphatic ring. In some embodiments, a formed ring is an optionally substituted 3-8 membered cycloaliphatic ring. In some embodiments, a formed ring is an optionally substituted 5-8 membered cycloaliphatic ring. In some embodiments, a formed ring is an optionally substituted 5-membered cycloaliphatic ring. In some embodiments, a formed ring is an optionally substituted 6-membered cycloaliphatic ring. In some embodiments, a formed ring is an optionally substituted 7-membered cycloaliphatic ring. In some embodiments, a formed ring is substituted. In some embodiments, a formed ring is not substituted. In some embodiments, a formed ring contains no additional unsaturation in addition to the double bond in C(═CHR^{RG2}) or C(═CR^{RG1}R^{RG2}).

In some embodiments, —C(═CHR^{RG2})—CHR^{RG4} or —C(═CR^{RG1}R^{RG2})—CR^{RG3}R^{RG4} is optionally substituted

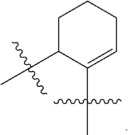 [SMILES: *C1=CCCCC1*]

In some embodiments, —C(═CHR^{RG2})—CHR^{RG4} or —C(═CR^{RG1}R^{RG2})—CR^{RG3}R^{RG4} is

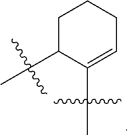 [SMILES: *C1=CCCCC1*]

In some embodiments, —C(═CHR^{RG2})—CHR^{RG4}-L^{RG3}- or —C(═CR^{RG1}R^{RG2})—CR^{RG3}R^{RG4}-L^{RG3}- is optionally substituted

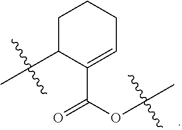 [SMILES: *OC(=O)C1=CCCCC1*]

In some embodiments, —C(═CHR^{RG2})—CHR^{RG4}-L^{RG3}- or —C(═CR^{RG1}R^{RG2})—CR^{RG3}R^{RG4}-L^{RG3}- is

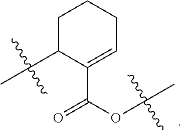 [SMILES: *OC(=O)C1=CCCCC1*]

In some embodiments, -L^{RG1}-C(═CHR^{RG2})—CHR^{RG4}-L^{RG3}- or -L^{RG1}-C(═CR^{RG1}R^{RG2}—CR^{RG3}R^{RG4}-L^{RG3}- is optionally substituted

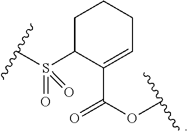 [SMILES: *OC(=O)C1=CCCCC1S(*)(=O)=O]

In some embodiments, -L^{RG1}-C(═CHR^{RG2})—CHR^{RG4}-L^{RG3}- or -L^{RG1}-C(═CR^{RG1}R^{RG2})—CR^{RG3}R^{RG4}-L^{RG3}- is optionally substituted

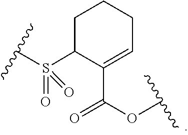 [SMILES: *OC(=O)C1=CCCCC1S(*)(=O)=O]

In some embodiments, a reactive group is a structure selected from the Table below. In some embodiments, -L^{LG2}-L^{LG3}-L^{LG4}-L^{RG1}-L^{RG2}- is a structure selected from the Table below. In some embodiments, -L^{LG2}-L^{LG3}-L^{LG4}-RG- is a structure selected from the Table below.

TABLE RG-1
Certain structures as examples.

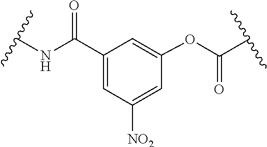 [SMILES: *NC(=O)c1cc(OC(*)=O)cc([N+](=O)[O-])c1]

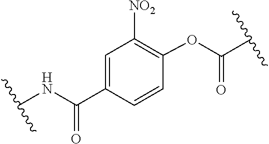 [SMILES: *NC(=O)c1ccc(OC(*)=O)c([N+](=O)[O-])c1]

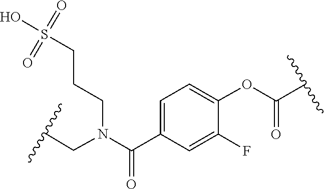 [SMILES: *CN(CCCS(=O)(=O)O)C(=O)c1ccc(OC(*)=O)c(F)c1]

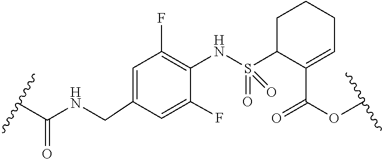 [SMILES: *OC(=O)C1=CCCCC1S(=O)(=O)Nc1c(F)cc(CNC(*)=O)cc1F]

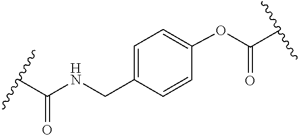 [SMILES: *C(=O)NCc1ccc(OC(*)=O)cc1]

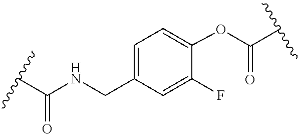 [SMILES: *C(=O)NCc1ccc(OC(*)=O)c(F)c1]

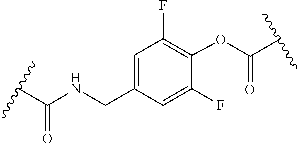 [SMILES: *C(=O)NCc1cc(F)c(OC(*)=O)c(F)c1]

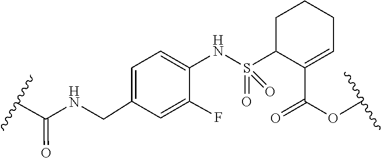 [SMILES: *OC(=O)C1=CCCCC1S(=O)(=O)Nc1ccc(CNC(*)=O)cc1F]

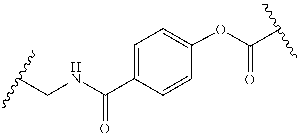 [SMILES: *CNC(=O)c1ccc(OC(*)=O)cc1]

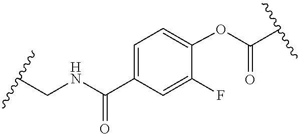 [SMILES: *CNC(=O)c1ccc(OC(*)=O)c(F)c1]

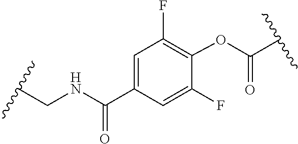 [SMILES: *CNC(=O)c1cc(F)c(OC(*)=O)c(F)c1]

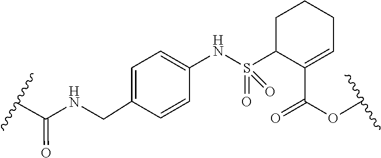 [SMILES: *OC(=O)C1=CCCCC1S(=O)(=O)Nc1ccc(CNC(*)=O)cc1]

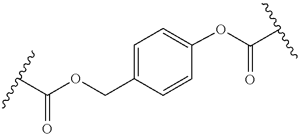 [SMILES: *C(=O)OCc1ccc(OC(*)=O)cc1]

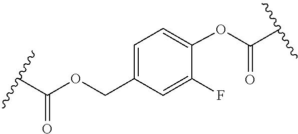 [SMILES: *C(=O)OCc1ccc(OC(*)=O)c(F)c1]

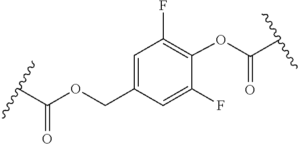 [SMILES: *C(=O)OCc1cc(F)c(OC(*)=O)c(F)c1]

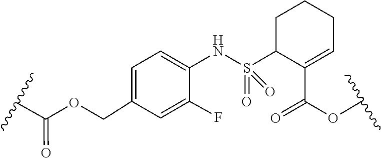 [SMILES: *OC(=O)C1=CCCCC1S(=O)(=O)Nc1ccc(COC(*)=O)cc1F]

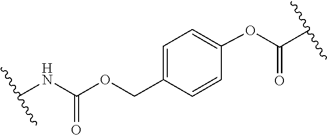 [SMILES: *NC(=O)OCc1ccc(OC(*)=O)cc1]

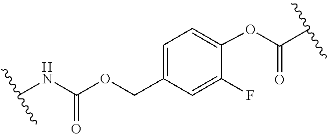 [SMILES: *NC(=O)OCc1ccc(OC(*)=O)c(F)c1]

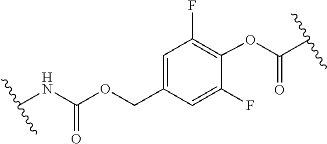 [SMILES: *NC(=O)OCc1cc(F)c(OC(*)=O)c(F)c1]

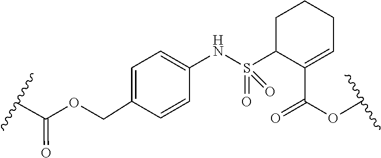 [SMILES: *OC(=O)C1=CCCCC1S(=O)(=O)Nc1ccc(COC(*)=O)cc1]

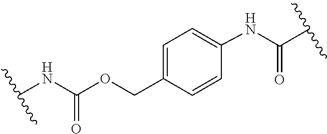 [SMILES: *NC(=O)OCc1ccc(NC(*)=O)cc1]

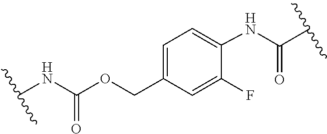 [SMILES: *NC(=O)OCc1ccc(NC(*)=O)c(F)c1]

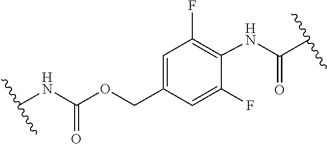 [SMILES: *NC(=O)OCc1cc(F)c(NC(*)=O)c(F)c1]

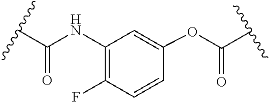 [SMILES: *C(=O)Nc1cc(OC(*)=O)ccc1F]

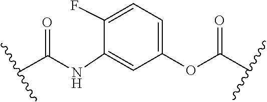 [SMILES: *C(=O)Nc1cc(OC(*)=O)ccc1F]

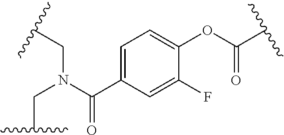 [SMILES: *CN(C*)C(=O)c1ccc(OC(*)=O)c(F)c1]

and

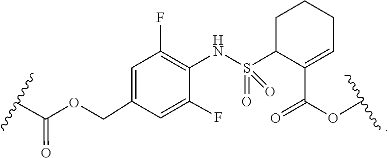 [SMILES: *OC(=O)C1=CCCCC1S(=O)(=O)Nc1c(F)cc(COC(*)=O)cc1F]

In some embodiments, -L^{LG4}-L^{RG2}- is —O—C(O)—. In some embodiments, -L^{LG4}-L^{RG2}- is —S—C(O)—. In some embodiments, -L^{LG4}-L^{RG1}-L^{RG2}- is —S—C(O)—.

In some embodiments, -L^{LG4}-L^{RG2}- is —N(—)—C(O)—, wherein N is a ring atom of an optionally substituted heteroaryl ring. In some embodiments, -L^{LG4}-L^{RG2}- is —N(—)—C(O)—, wherein N is a ring atom of L^{LG4} which is or comprises an optionally substituted heteroaryl ring. In some embodiments, -L^{LG4}-L^{RG2}- is —N(—)—C(O)—O—, wherein N is a ring atom of L^{LG4} which is or comprises an optionally substituted heteroaryl ring.

In some embodiments, L^{RG2} is optionally substituted —CH₂—C(O)—, wherein —CH₂— is bonded to an electron-withdrawing group comprising or connected to a target binding moiety. In some embodiments, L^{RG2} is optionally substituted —CH₂— bonded to an electron-withdrawing group comprising or connected to a target binding moiety. In some embodiments, L^{RG1} is an electron-withdrawing group. In some embodiments, L^{RG1} is —C(O)—. In some embodiments, L^{RG1} is —S(O)—. In some embodiments, L^{RG1} is —S(O)₂—. In some embodiments, L^{RG1} is —P(O(OR)—. In some embodiments, L^{RG1} is —P(O(SR)—. In some embodiments, L^{RG1} is —P(O(N(R)₂)—. In some embodiments, L^{RG1} is —OP(O(OR)—. In some embodiments, L^{RG1} is —OP(O(SR)—. In some embodiments, L^{RG1} is —OP(O(N(R)₂)—.

In some embodiments, L^{RG2} is optionally substituted —CH₂—C(O)—, wherein —CH₂— is bonded to a leaving group comprising or connected to a target binding moiety. In some embodiments, L^{RG2} is optionally substituted —CH₂— bonded to a leaving group comprising or connected to a target binding moiety. In some embodiments, L^{RG1} is —O—C(O)—. In some embodiments, L^{RG1} is —OS(O)₂—. In some embodiments, L^{RG1} is —OP(O(OR)—. In some embodiments, L^{RG1} is —OP(O(SR)—. In some embodiments, L^{RG1} is —OP(O(N(R)₂)—.

In some embodiments, a reactive group reacts with an amino group of a target agent. In some embodiments, an amino group is —NH₂ of the side chain of a lysine residue.

In some embodiments, a target agent is a protein agent. In some embodiments, a target agent is an antibody agent. In some embodiments, a reactive group reacts with an amino acid residue of such protein or antibody agent. In some embodiments, an amino acid residue is a lysine residue. In some embodiments, a reactive group reacts with —NH₂ of the side chain of a lysine residue. In some embodiments, a reactive group is or comprises —C(O)—O—, it reacts with —NH₂ (e.g., of the side chain of a lysine residue), and forms an amide group —C(O)—O— with the —NH₂.

### Linker Moieties

In some embodiments, moieties are optionally connected to each other through linker moieties. For example, in some embodiments, a reactive group, e.g., RG, is connected to a moiety of interest, e.g., MOI, through a linker, e.g., L^{RM}. In some embodiments, a moiety, e.g., LG, may also comprise one or more linkers, e.g., L^{LG1}, L^{LG2}, L^{LG3}, L^{LG4}, etc., to link various portions. In some embodiments, L^{LG} is a linker moiety described herein. In some embodiments, L^{LG1} is a linker moiety described herein. In some embodiments, L^{LG2} is a linker moiety described herein. In some embodiments, L^{LG3} is a linker moiety described herein. In some embodiments, L^{LG4} is a linker moiety described herein. In some embodiments, L^{RM} is a linker moiety described herein. In some embodiments, L^{PM} is L as described herein. In some embodiments, L^{PM} is a linker moiety described herein. In some embodiments, L^{PM} is L as described herein.

Linker moieties of various types and/or for various purposes, e.g., those utilized in antibody-drug conjugates, etc., may be utilized in accordance with the present disclosure.

Linker moieties can be either bivalent or polyvalent depending on how they are used. In some embodiments, a linker moiety is bivalent. In some embodiments, a linker is polyvalent and connecting more than two moieties.

In some embodiments, a linker moiety, e.g., L^{z} (wherein z represents superscript text; e.g., L^{PM}, L^{RM}, L^{LG}, L^{LG1}, etc.), is or comprises L.

In some embodiments, L is a covalent bond, or a bivalent or polyvalent optionally substituted, linear or branched C₁₋₁₀₀ group comprising one or more aliphatic, aryl, heteroaliphatic having 1-20 heteroatoms, heteroaromatic having 1-20 heteroatoms, or any combinations thereof, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or -[(—O—C(R′)₂—C(R′)₂-)ₙ]-, wherein n is 1-20. In some embodiments, each amino acid residue is independently a residue of an amino acid having the structure of formula A-I or a salt thereof. In some embodiments, each amino acid residue independently has the structure of —N(R^{a1})-L^{a1}-C(R^{a2})(R^{a3})-L^{a2}-CO— or a salt form thereof.

In some embodiments, L is bivalent. In some embodiments, L is a covalent bond.

In some embodiments, L is a bivalent or optionally substituted, linear or branched group selected from C₁₋₀₀ aliphatic and C₁₋₁₀₀ heteroaliphatic having 1-50 heteroatoms, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue or -[(—O—C(R′)₂—C(R′)₂-)ₙ]-.

In some embodiments, L is a bivalent or optionally substituted, linear or branched group selected from C₁₋₂₀ aliphatic and C₁₋₂₀ heteroaliphatic having 1-10 heteroatoms, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue or -[(—O—C(R′)₂—C(R′)₂-)ₙ]-.

In some embodiments, L is a bivalent or optionally substituted, linear or branched group selected from C₁₋₂₀ aliphatic wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue or -[(—O—C(R′)₂—C(R′)₂-)ₙ]-.

In some embodiments, L is a bivalent or optionally substituted, linear or branched group selected from C₁₋₂₀ aliphatic wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, an amino acid residue or -[(—O—C(R′)₂—C(R′)₂-)ₙ]-.

In some embodiments, L is a bivalent or optionally substituted, linear or branched group selected from C₁₋₂₀ aliphatic wherein one or more methylene units of the group are optionally and independently replaced with —C≡C—, -Cy-, —C(R′)₂—, —O—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, an amino acid residue or -[(—O—C(R′)₂—C(R′)₂-)ₙ]-.

In some embodiments, a linker moiety, e.g., L, L^{PM}, L^{RM}, etc., comprises an acidic group, e.g., —S(O)₂OH.

In some embodiments, L is or comprises -[(—O—C(R′)₂—C(R′)₂-)ₙ]-. In some embodiments, L is or comprises -[(—O—CH₂—CH₂-)ₙ]-. In some embodiments, L is -[(—CH₂—CH₂-0)₆]—CH₂—CH₂—. In some embodiments, L is -[(—CH₂—CH₂—O)₈]—CH₂—CH₂—. In some embodiments, —CH₂—CH₂—O—is bonded to a target binding moiety at a —CH₂—. In some embodiments, —CH₂—CH₂—O—is bonded to a moiety of interest at a —CH₂—. In some embodiments, L^{PM} is such L as described herein. In some embodiments, L^{RM} is such L as described herein.

In some embodiments, a linker moiety is trivalent or polyvalent. For example, in some embodiments, a linker moiety is L as described herein and L is trivalent or polyvalent. In some embodiments, L is trivalent. For example, in some embodiments, L is —CH₂—N(—CH₂—)—C(O)—.

In some embodiments, L is or comprises a bioorthogonal or enzymatic reaction product moiety. In some embodiments, L is or comprise an optionally substituted triazole moiety (which is optionally part of a bi- or poly-cyclic ring system). In some embodiments, L is or comprises LPXTG. In some embodiments, L is or comprises LPETG. In some embodiments, L is or comprises LPXT(G)ₙ, wherein n is 1, 2, 3, 4, 5, 6, 7, 8, 9, or 10. In some embodiments, L is or comprises LPET(G)ₙ, wherein n is 1, 2, 3, 4, 5, 6, 7, 8, 9, or 10.

In some embodiments, provided compounds/agents (e.g., reaction partners, agents (e.g., products of provided methods and/or steps therein) comprise no cleavable groups (except one or more reactive groups and/or moieties therein) that could be cleaved under conditions that would not substantially damage or transform target agents and/or agents comprising target agent moieties (e.g., conjugation products comprising target agent moieties). In some embodiments, provided compounds/agents (e.g., reaction partners, agents (e.g., products of provided methods and/or steps therein) comprise no cleavable groups (except one or more reactive groups and/or moieties therein) that could be cleaved under conditions that would not render target agents and/or agents comprising target agent moieties (e.g., conjugation products comprising target agent moieties) ineffective for one or more uses (e.g., for use as diagnostic agents, therapeutic agents, etc.). In some embodiments, provided compounds/agents (e.g., reaction partners, agents (e.g., products of provided methods and/or steps therein) comprise no cleavable groups which can be cleaved under bioorthogonal conditions. In some embodiments, provided compounds/agents (e.g., reaction partners, agents (e.g., products of provided methods and/or steps therein) comprise no cleavable groups which can be cleaved without substantively damaging and/or transforming proteins. In some embodiments, a cleavable group is or comprises —S—, —S—S—, —S-Cy-, —C(O)—O—, —C(O)—S—, acetal moiety, —N═N—, imine moiety, —CH═N—, —P(O)(OR)O— moiety, —P(O)(OR)—N(R)— moiety, —C(O)—CH₂—C(COOH)═CHC(O)— moiety, —CHOH—CHOH-moiety, —Se— moiety, Si bonded to two oxygen atoms, —C(O)—CH₂— wherein the —CH₂—is bonded to a benzylic carbon wherein the phenyl ring of the benzyl group is substituted with —NO₂—, —C(O)—CH₂— wherein the —CH₂—is bonded to a benzylic carbon wherein the phenyl ring of the benzyl group is substituted with —NO₂— at o-position, or —C(O)—N(—)- moiety, wherein N is a ring atom of a heteroaryl ring. In some embodiments, a cleavable group is or comprises —S—S—, —S—CH₂—Cy-, —S-Cy-, —C(O)—O—, —C(O)—S—, acetal moiety, —N═N—, imine moiety, —CH═N—, —P(O)(OR)O— moiety, —P(O)(OR)—N(R)— moiety, —C(O)—CH₂—C(COOH)═CHC(O)— moiety, —CHOH—CHOH— moiety, —Se— moiety, Si bonded to two oxygen atoms, —C(O)—CH₂— wherein the —CH₂—is bonded to a benzylic carbon wherein the phenyl ring of the benzyl group is substituted with —NO₂—, —C(O)—CH₂— wherein the —CH₂—is bonded to a benzylic carbon wherein the phenyl ring of the benzyl group is substituted with —NO₂— at o-position, or —C(O)—N(-)-moiety, wherein N is a ring atom of a heteroaryl ring.

In some embodiments, a linker moiety does not contain a cleavage group above. In some embodiments, a linker moiety does not contain one or more or any of the following moieties: —S—, —S—S—, —S—CH₂—Cy-, —S-Cy-, —C(O)—O—, —C(O)—S—, acetal moiety, —N═N—, imine moiety, —CH═N—, —P(O)(OR)O— moiety, —P(O)(OR)—N(R)— moiety, —C(O)—CH₂—C(COOH)═CHC(O)— moiety, —CHOH—CHOH-moiety, —Se— moiety, Si bonded to two oxygen atoms, —C(O)—CH₂— wherein the —CH₂—is bonded to a benzylic carbon wherein the phenyl ring of the benzyl group is substituted with —NO₂—, —C(O)—CH₂— wherein the —CH₂—is bonded to a benzylic carbon wherein the phenyl ring of the benzyl group is substituted with —NO₂— at o-position, or —C(O)—N(—)- moiety, wherein N is a ring atom of a heteroaryl ring. In some embodiments, a linker moiety does not contain one or more or any of the following moieties: —S—S—, —S—CH₂—Cy-, —S-Cy-, —C(O)—O—, —C(O)—S—, acetal moiety, —N═N—, imine moiety, —CH═N—, —P(O)(OR)O— moiety, —P(O)(OR)—N(R)— moiety, —C(O)—CH₂—C(COOH)═CHC(O)— moiety, —CHOH—CHOH— moiety, —Se— moiety, Si bonded to two oxygen atoms, —C(O)—CH₂— wherein the —CH₂—is bonded to a benzylic carbon wherein the phenyl ring of the benzyl group is substituted with —NO₂—, —C(O)—CH₂— wherein the —CH₂—is bonded to a benzylic carbon wherein the phenyl ring of the benzyl group is substituted with —NO₂— at o-position, or —C(O)—N(—)- moiety, wherein N is a ring atom of a heteroaryl ring. In some embodiments, a linker moiety comprises no -5-. In some embodiments, a linker moiety comprises no —S—S— (optionally except a disulfide moiety formed by two amino acid residues, in some embodiments, optionally except a disulfide moiety formed by two cysteine residues).

In some embodiments, a linker moiety comprises no —S-Cy-. In some embodiments, a linker moiety comprises no —S—CH₂—Cy-. In some embodiments, a linker moiety comprises no —C(O)—O—. In some embodiments, a linker moiety comprises no —C(O)—S—. In some embodiments, a linker moiety comprises no acetal moiety. In some embodiments, a linker moiety comprises no —N═N—. In some embodiments, a linker moiety comprises no imine moiety. In some embodiments, a linker moiety comprises no —CH═N-(optionally except in a ring, in some embodiments, optionally except in a heteroaryl ring). In some embodiments, a linker moiety comprises no —P(O)(OR)O— moiety. In some embodiments, a linker moiety comprises no —P(O)(OR)—N(R)— moiety. In some embodiments, a linker moiety comprises no —C(O)—CH₂—C(COOH)═CHC(O)— moiety. In some embodiments, a linker moiety comprises no —CHOH—CHOH— moiety. In some embodiments, a linker moiety comprises no —Se— moiety. In some embodiments, a linker moiety comprises no Si bonded to two oxygen atoms. In some embodiments, a linker moiety comprises no —C(O)—CH₂—, wherein the —CH₂—is bonded to a benzylic carbon, wherein the phenyl ring of the benzyl group is substituted with —NO₂—. In some embodiments, a linker moiety comprises no —C(O)—CH₂—, wherein the —CH₂—is bonded to a benzylic carbon, wherein the phenyl ring of the benzyl group is substituted with —NO₂— at o-position. In some embodiments, a linker moiety comprise no —C(O)—N(—)- moiety, wherein N is a ring atom of a heteroaryl ring. In some embodiments, a linker moiety does not contain any of these groups. In some embodiments, L^{RM} is such a linker moiety. In some embodiments, L^{PM} is such a linker moiety. In some embodiments, L^{LG} is such a linker moiety. In some embodiments, an agent of the present disclosure does not contain one or more or all of such moieties.

In some embodiments, L is a covalent bond. In some embodiments, L is a bivalent optionally substituted, linear or branched C₁₋₁₀₀ aliphatic group wherein one or more methylene units of the group are optionally and independently replaced. In some embodiments, L is a bivalent optionally substituted, linear or branched C₆₋₁₀₀ arylaliphatic group wherein one or more methylene units of the group are optionally and independently replaced. In some embodiments, L is a bivalent optionally substituted, linear or branched C₅₋₁₀₀ heteroarylaliphatic group having 1-20 heteroatoms wherein one or more methylene units of the group are optionally and independently replaced. In some embodiments, L is a bivalent optionally substituted, linear or branched C₁₋₁₀₀ heteroaliphatic group having 1-20 heteroatoms wherein one or more methylene units of the group are optionally and independently replaced.

In some embodiments, a linker moiety (e.g., L) is or comprises one or more (e.g., 1, 2, 3, 4, 5, 6, 7, 8, 9, 10, 11, 12, 13, 14, 15, 16, 17, 18, 19, 20 or more) polyethylene glycol units. In some embodiments, a linker moiety is or comprises —(CH₂CH₂O)ₙ—, wherein n is as described in the present disclosure. In some embodiments, one or more methylene units of L are independently replaced with —(CH₂CH₂O)ₙ—.

As described herein, in some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, n is 11. In some embodiments, n is 12. In some embodiments, n is 13. In some embodiments, n is 14. In some embodiments, n is 15. In some embodiments, n is 16. In some embodiments, n is 17. In some embodiments, n is 18. In some embodiments, n is 19. In some embodiments, n is 20.

In some embodiments, a linker moiety (e.g., L) is or comprises one or more (e.g., 1, 2, 3, 4, 5, 6, 7, 8, 9, 10, 11, 12, 13, 14, 15, 16, 17, 18, 19, 20 or more) amino acid residues. As used in the present disclosure, “one or more” can be 1-100, 1-50, 1-40, 1-30, 1-20, 1-10, 1-5, 1, 2, 3, 4, 5, 6, 7, 8, 9, 10, 11, 12, 13, 14, 15, 16, 17, 18, 19, or 20 or more. In some embodiments, one or more methylene units of L are independently replaced with an amino acid residue. In some embodiments, one or more methylene units of L are independently replaced with an amino acid residue, wherein the amino acid residue is of an amino acid of formula A-I or a salt thereof. In some embodiments, one or more methylene units of L are independently replaced with an amino acid residue, wherein each amino acid residue independently has the structure of —N(R^{a1})-L^{a1}-C(R^{a2})(R^{a3})-L^{a2}-CO— or a salt form thereof.

In some embodiments, a linker moiety comprises one or more moieties, e.g., amino, carbonyl, etc., that can be utilized for connection with other moieties. In some embodiments, a linker moiety comprises one or more —NR′—, wherein R′ is as described in the present disclosure. In some embodiments, —NR′— improves solubility. In some embodiments, —NR′— serves as connection points to another moiety. In some embodiments, R′ is —H. In some embodiments, one or more methylene units of L are independently replaced with —NR′—, wherein R′ is as described in the present disclosure.

In some embodiments, a linker moiety, e.g., L, comprises a —C(O)— group, which can be utilized for connections with a moiety. In some embodiments, one or more methylene units of L are independently replaced with —C(O)—.

In some embodiments, a linker moiety, e.g., L, comprises a —NR′— group, which can be utilized for connections with a moiety. In some embodiments, one or more methylene units of L are independently replaced with —N(R′)—.

In some embodiments, a linker moiety, e.g., L, comprises a —C(O)NR′— group, which can be utilized for connections with a moiety. In some embodiments, one or more methylene units of L are independently replaced with —C(O)N(R′)—.

In some embodiments, a linker moiety, e.g., L, comprises a —C(R′)₂— group. In some embodiments, one or more methylene units of L are independently replaced with —C(R′)₂—. In some embodiments, —C(R′)₂—is —CHR′—. In some embodiments, R′ is —(CH₂)₂C(O)NH(CH₂)₁₁COOH. In some embodiments, R′ is —(CH₂)₂COOH. In some embodiments, R′ is —COOH.

In some embodiments, a linker moiety is or comprises one or more ring moieties, e.g., one or more methylene units of L are replaced with -Cy-. In some embodiments, a linker moiety, e.g., L, comprises an aryl ring. In some embodiments, a linker moiety, e.g., L, comprises an heteroaryl ring. In some embodiments, a linker moiety, e.g., L, comprises an aliphatic ring. In some embodiments, a linker moiety, e.g., L, comprises an heterocyclyl ring. In some embodiments, a linker moiety, e.g., L, comprises a polycyclic ring. In some embodiments, a ring in a linker moiety, e.g., L, is 3-20 membered. In some embodiments, a ring is 5-membered. In some embodiments, a ring is 6-membered. In some embodiments, a ring in a linker is product of a cycloaddition reaction (e.g., click chemistry, and variants thereof) utilized to link different moieties together.

In some embodiments, a linker moiety (e.g., L) is or comprises

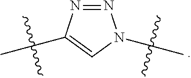 [SMILES: *c1cn(*)nn1]

In some embodiments, a methylene unit of L is replaced with

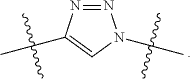 [SMILES: *c1cn(*)nn1]

In some embodiments, a methylene unit of L is replaced with -Cy-. In some embodiments, -Cy- is

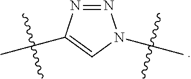 [SMILES: *c1cn(*)nn1]

In some embodiments, a linker moiety (e.g., L) is or comprises -Cy-. In some embodiments, a methylene unit of L is replaced with -Cy-. In some embodiments, -Cy- is

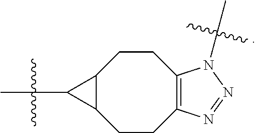 [SMILES: *C1C2CCc3nnn(*)c3CCC12]

In some embodiments, -Cy- is

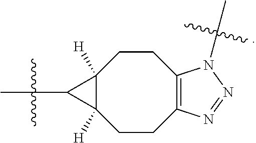 [SMILES: *C1[C@H]2CCc3c(nnn3*)CC[C@@H]12]

In some embodiments, -Cy- is

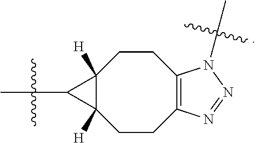 [SMILES: *C1[C@H]2CCc3nnn(*)c3CC[C@@H]12]

In some embodiments, a linker moiety, e.g., L, comprises

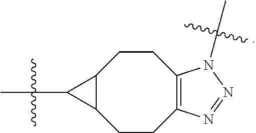 [SMILES: *C1C2CCc3nnn(*)c3CCC12]

In some embodiments, L^{RM} is a covalent bond. In some embodiments, L^{RM} is not a covalent bond. In some embodiments, L^{RM} is or comprises —(CH₂CH₂O)ₙ—. In some embodiments, L^{RM} is or comprises —(CH₂)ₙ—O—(CH₂CH₂O)ₙ—(CH₂)ₙ—, wherein each n is independently as described herein, and each —CH₂— is independently optionally substituted. In some embodiments, L^{RM} is —(CH₂)ₙ—O—(CH₂CH₂O)ₙ—(CH₂)ₙ—, wherein each n is independently as described herein, and each —CH₂— is independently optionally substituted. In some embodiments, L^{RM} is —(CH₂)₂—O—(CH₂CH₂O)ₙ—(CH₂)₂—, wherein n is as described herein, and each —CH₂— is independently optionally substituted. In some embodiments, L^{RM} is —(CH₂)₂—O—(CH₂CH₂O)ₙ—(CH₂)₂—, wherein n is as described herein.

In some embodiments, L^{PM} is a covalent bond. In some embodiments, L^{PM} is not a covalent bond. In some embodiments, L^{PM} is or comprises —(CH₂CH₂O)ₙ—. In some embodiments, L^{PM} is or comprises —(CH₂)ₙ—O—(CH₂CH₂O)ₙ—(CH₂)ₙ—, wherein each n is independently as described herein, and each —CH₂— is independently optionally substituted. In some embodiments, L^{PM} is —(CH₂)ₙ—O—(CH₂CH₂O)ₙ—(CH₂)ₙ—, wherein each n is independently as described herein, and each —CH₂— is independently optionally substituted. In some embodiments, L^{PM} is —(CH₂)₂—O—(CH₂CH₂O)ₙ—(CH₂)₂—, wherein n is as described herein, and each —CH₂— is independently optionally substituted. In some embodiments, L^{PM} is —(CH₂)₂—O—(CH₂CH₂O)ₙ—(CH₂)₂—, wherein n is as described herein.

In some embodiments, L^{PM} (e.g., in a product of a first and a second agents) is or comprises a reaction product moiety formed a first reactive moiety and a second reactive moiety.

In some embodiments, a linker moiety (e.g., L^{PM} in a product of a first and a second agents) is or comprises

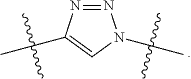 [SMILES: *c1cn(*)nn1]

In some embodiments, a methylene unit of a linker moiety, e.g., L or a linker moiety that can be L (e.g., L^{RM}, L^{PM}, etc.) is replaced with -Cy-. In some embodiments, -Cy- is optionally substituted

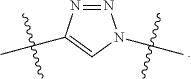 [SMILES: *c1cn(*)nn1]

In some embodiments, -Cy- is

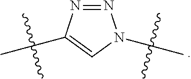 [SMILES: *c1cn(*)nn1]

In some embodiments, -Cy- is

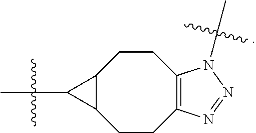 [SMILES: *C1C2CCc3nnn(*)c3CCC12]

In some embodiments, -Cy- is

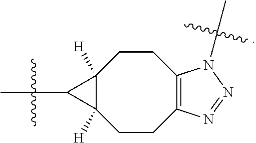 [SMILES: *C1[C@H]2CCc3c(nnn3*)CC[C@@H]12]

In some embodiments, -Cy- is

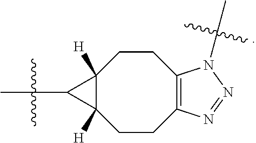 [SMILES: *C1[C@H]2CCc3nnn(*)c3CC[C@@H]12]

### Conjugates

In some embodiments, provided technologies comprise contacting a target agent (e.g., to which RBM is to be attached) with a reaction partner. In some embodiments, contact is performed under conditions and for a time so that a target agent react with a reaction partner to form an agent as a product. Many reaction conditions/reaction times in the art may be assessed and utilized if suitable for desired purposes in accordance with the present disclosure; certain such conditions, reaction times, assessment, etc. are described in the Examples.

In some embodiments, an agent formed comprises a target agent moiety, a moiety of interest and optionally a linker moiety connecting a target agent moiety and a moiety of interest. In some embodiments, a target agent moiety is derived from a target agent (e.g., by removing one or more —H from a target agent). In some embodiments, a target agent moiety maintains one or more, most, or substantially all structural features and/or biological functions of a target agent. For example, in some embodiments, a target agent is an antibody agent, and a target agent moiety in a formed agent is a corresponding antibody agent moiety and maintains major functions of the antibody agent, e.g., interacting with various receptors (e.g., Fc receptors such as FcRn), recognizing antigen with specificity, triggering, promoting, and/or enhancing immunological activities toward diseased cells, etc., as the antibody agent. In some embodiments, a formed agent provides one or more functions beyond those of a target agent, for example, from a moiety of interest and/or a formed agent as a whole.

In some embodiments, an agent formed has the structure of formula P-1 or P-II, or a salt thereof. In some embodiments, a moiety of interest in a formed agent (e.g., RBM of formula P-1 or P-II, or a salt thereof) is the same as a moiety of interest in a reaction partner (e.g., RBM of formula R—I or a salt thereof) utilized to prepare a formed agent. In some embodiments, P is a protein moiety. In some embodiments, P is an antibody moiety.

In some embodiments, linker moieties (or a part thereof) connected to moieties of interest may also be transferred from reaction partners (e.g., L^{RM} of formula R—I or a salt thereof). In some embodiments, a linker moiety in a formed agent (e.g., L^{PM}) is or comprises a linker moiety in a reaction partner (e.g., one between a reactive group and a moiety of interest, e.g., L^{RM}). In some embodiments, L^{PM} is or comprises L^{RM}. In some embodiments, L^{PM} is -L^{RM}-L^{RG2}-. In some embodiments, L^{RG2} is —C(O)—. In some embodiments, L^{RG2} is —C(O)—, and is bonded to —NH— of a target agent moiety, e.g., —NH— in a side chain of a lysine residue of a protein moiety, which in some embodiments, is an antibody moiety.

Reaction partners, e.g., compounds of formula R—I or salts thereof, typically do not contain moieties that can react with reactive groups under conditions under which reactive groups react with target agents. In some embodiments, to the extent that some moieties in reaction partners may react with reactive groups under conditions under which reactive groups react with target agents, reactions between such moieties and reactive groups are significantly slower and/or less efficient compared to reactions between reactive groups and target agents. In some embodiments, reactions between such moieties and reactive groups do not significantly reduce (e.g., no more than about 5%, 10%, 15%, 20%, 25%, 30%, 40%, 50%, 60%, 70%, 80%, 90%, etc. of reduction) efficiencies, yields, rates, and/or conversions, etc., of reactions between reactive groups and target agents. In some embodiments, reactive groups (e.g., ester groups, activated carboxylic acid derivatives, etc.) react with amino groups (e.g., —NH₂ groups) of target agents (e.g., protein agents such as antibody agents). In some embodiments, reaction partners, e.g., compounds of formula R—I or salts thereof, do not contain amine groups. In some embodiments, compounds of formula R—I or salts thereof (or portions thereof, such as R^{L}G, L^{L}G, L^{LG1}, L^{LG2}, L^{LG3}, L^{LG4}, L^{RG1}, L^{RG2}, L^{RM}, and/or MOI) do not contain amine groups. In some embodiments, they do not contain primary amine groups (—NH₂). In some embodiments, they do not contain —CH₂NH₂. In some embodiments, they do not contain —CH₂CH₂NH₂. In some embodiments, they do not contain —CH₂CH₂CH₂NH₂. In some embodiments, they do not contain —CH₂CH₂CH₂CH₂NH₂. In some embodiments, amine groups, e.g., primary amine groups, are capped (e.g., by introduction of acyl groups (e.g., R—C(O)— (e.g., acetyl)) to form amide groups) to prevent or reduce undesired reactions.

In some embodiments, reactions are performed in buffer systems. In some embodiments, buffer systems of present disclosure maintain structures and/or functions of target agents, moiety of interest, etc. In some embodiments, a buffer is a phosphate buffer. In some embodiments, a buffer is a PBS buffer. In some embodiments, a buffer is a borate buffer. In some embodiments, buffers of the present disclosure provide and optionally maintain certain pH value or range. For example, in some embodiments, a useful pH is about 7-9, e.g., 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 9.0, etc. In some embodiments, a pH is 7.4. In some embodiments, a pH is 7.5. In some embodiments, a pH is 7.8. In some embodiments, a pH is 8.0. In some embodiments, a pH is 8.2. In some embodiments, a pH is 8.3.

Provided technologies can provide various advantages. Among other things, in some embodiments, connection of a moiety of interest in a provided reaction partner (e.g., a compound comprising a reactive group located between a first group and a moiety of interest (e.g., a compound of formula R—I or a salt thereof)) to a target agent and release of a target binding moiety in a provided reaction partner can be achieved in one reaction and/or in one pot. Thus, in many embodiments, no separate reactions/steps are performed to remove target binding moieties. As appreciated by those skilled in the art, by performing connection of moiety of interest and release of target binding moiety in a single reaction/operation, provided technologies can avoid separate steps for target binding moiety removal and can improve overall efficiency (e.g., by simplify operations, increasing overall yield, etc.), reduce manufacturing cost, improve product purity (e.g., by avoiding exposure to target binding moiety removal conditions, which typically involve one or more of reduction, oxidation, hydrolysis (e.g., of ester groups), etc., conditions and may damage target agent moieties (e.g., for protein agent moieties, protein amino acid residues, overall structures, and/or post-translational modifications (e.g., glycans of antibodies) thereof. Indeed, as demonstrated herein, provided technologies among other things can provided improved efficiency (e.g., in terms of reaction rates and/or conversion percentages), increased yield, increased purity/homogeneity, and/or enhanced selectivity, particularly compared to reference technologies wherein a reaction partner containing no target binding moieties is used, without introducing step(s) for target binding moiety removal (e.g., target binding moiety is removed in the same step as moiety of interest conjugation).

In some embodiments, the present disclosure provides products of provided processes, which, among other things, contain low levels of damage to target agent moieties compared to processes comprising steps which are performed for target binding moiety removal but not for substantial moiety of interest conjugation. In some embodiments, provided product compositions have high homogeneity compared to reference product compositions (e.g., those from technologies without using target binding moieties, or utilizing extra step(s) for target binding moiety removal (e.g., not utilizing reaction partners described herein which comprise a reactive group located between a target binding moiety and a moiety of interest).

In some embodiments, a product agent is an agent comprising:
a target agent moiety;
a moiety of interest, such as MMAD; and
optionally one or more linker moieties.

In some embodiments, a target agent moiety is a protein agent moiety. In some embodiments, a target agent moiety is an antibody agent moiety. In some embodiments, an antibody agent moiety comprises IgG Fc region. In some embodiments, a target agent moiety is connected to a moiety of interest through an amino group optionally through a linker. In some embodiments, it is through a lysine residue wherein the amino group of the side chain is connected to a moiety of interest optionally through a linker (e.g., forming —NH—C(O)— as part of an amide group, a carbamate group, etc.).

In some embodiments, selected locations of target agents are utilized for conjugation. For example, in some embodiments, K246 or K248 of an antibody agent (EU numbering, or corresponding residues) are conjugation locations. In some embodiments, a conjugation location is K246 of heavy chain (unless otherwise specified, locations herein include corresponding residues in, e.g., modified sequence (e.g., longer, shorter, rearranged, etc., sequences)). In some embodiments, a location is K248 of heavy chain. In some embodiments, a location is K288 or K290 of heavy chain. In some embodiments, a location is K288 of heavy chain. In some embodiments, a location is K290 of heavy chain. In some embodiments, a location is K317.

In some embodiments, when target agents are antibody agents, heavy chains are selectively labeled over light chains.

Among other things, the present disclosure can provide controlled moiety of interest/target agent ratios (e.g., for antibody-drug conjugates, drug/antibody ratio (DAR)). In some embodiments, a ratio is about 0.5-6, e.g., 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 4.5, 5.0, 5.5, etc.). In some embodiments, a ratio is about 0.5-2.5. In some embodiments, a ratio is about 0.5-2. In some embodiments, a ratio is about 1-2. In some embodiments, a ratio is about 1.5-2. In some embodiments, a ratio is of moieties of interest conjugated to target agent moieties and target agent moieties conjugated to moieties of interest. In some embodiments, a ratio is of moieties of interest conjugated to target agent moieties and all target agent moieties in a composition.

In some embodiments, in provided agents (e.g., agents of formula P—I or P-II, or a salt thereof) substantially all conjugation sites of target agent moieties have the same modifications (e.g., all share the same moieties of interest optionally connected through the same linker moieties). In some embodiments, no conjugation sites bear different modifications (e.g., different moieties of interest and/or no moieties of interest and/or different linker moieties).

In some embodiments, in provided compositions comprising a plurality of provided agents (e.g., agents of formula P—I or P-II, or a salt thereof) substantially all conjugation sites of target agent moieties have the same modifications (e.g., all share the same moieties of interest optionally connected through the same linker moieties). In some embodiments, no conjugation sites bear different modifications (e.g., different moieties of interest and/or no moieties of interest and/or different linker moieties). In some embodiments, such compositions do not contain agents that share the same (or substantially the same) target agent moieties but different modifications (e.g., different moieties of interest and/or no moieties of interest and/or different linker moieties). In some embodiments, agents that share the same (or substantially the same) target agent moieties but different modifications (e.g., different moieties of interest and/or no moieties of interest and/or different linker moieties) are intermediates of multiple-step preparations (e.g., comprising steps for removal of target binding moieties in addition to steps for moiety of interest conjugation) of final product agents.

In some embodiments, the present disclosure provides a composition comprising a plurality of agents each of which independently comprising:
a target agent moiety,
a moiety of interest, and
optionally a linker moiety linking a target agent moiety and a moiety of interest;
   wherein agents of the plurality share the same or substantially the same target agent moiety, and a common modification independently at at least one common location; and
wherein about 1%-100% of all agents that comprise a target agent moiety and a moiety of interest are agents of the plurality.

In some embodiments, a target agent moiety is or comprises a protein moiety. In some embodiments, agents of the plurality share common modifications (e.g., conjugations of moieties of interest optionally through linker moieties) independently at at least one amino acid residues. In some embodiments, agents of the plurality are each independently of formula P—I or P-II, or a salt thereof.

In some embodiments, the present disclosure provides a composition comprising a plurality of agents each of which independently comprising:
a protein agent moiety,
a moiety of interest, and
optionally a linker moiety linking the protein agent moiety and a moiety of interest;
   wherein protein agent moieties of agents of the plurality comprise a common amino acid sequence, and agents of the plurality share a common modification independently at at least one common amino acid residue of protein agent moieties; and
wherein about 1%-100% of all agents that comprise a protein agent moiety that comprise the common amino acid sequence and a moiety of interest are agents of the plurality.

In some embodiments, agents of the plurality are each independently of formula P-1 or P-II, or a salt thereof. In some embodiments, each protein agent moiety is independently an antibody agent moiety.

In some embodiments, the present disclosure provides a composition comprising a plurality of agents each of which independently comprising:
an antibody agent moiety,
a moiety of interest, and
optionally a linker moiety linking an antibody agent moiety and a moiety of interest; wherein antibody agent moieties of agents of the plurality comprise a common amino acid sequence or can bind to a common antigen, and agents of the plurality share a common modification independently at at least one common amino acid residue of protein agent moieties; and
wherein about 1%-100% of all agents that comprise an antibody agent moiety that comprise the common amino acid sequence or can bind to the common antigen and a moiety of interest are agents of the plurality.

In some embodiments, agents of the plurality are each independently of formula P-1 or P-II, or a salt thereof. In some embodiments, antibody agent moieties of agents of the plurality comprise a common amino acid sequence. In some embodiments, antibody agent moieties of agents of the plurality comprise a common amino acid sequence in a Fc region. In some embodiments, antibody agent moieties of agents of the plurality comprise a common Fc region. In some embodiments, antibody agent moieties of agents of the plurality can bind a common antigen specifically. In some embodiments, antibody agent moieties are monoclonal antibody moieties. In some embodiments, antibody agent moieties are polyclonal antibody moieties. In some embodiments, antibody agent moieties bind to two or more different antigens. In some embodiments, antibody agent moieties bind to two or more different proteins. In some embodiments, antibody agent moieties are IVIG moieties.

As used in the present disclosure, in some embodiments, “at least one” or “one or more” is 1-1000, 1-500, 1-200, 1-100, 1-90, 1-80, 1-70, 1-60, 1-50, 1-40, 1-30, 1-20, 1-10, 1-5, or about 1, 2, 3, 4, 5, 6, 7, 8, 9, 10, 11, 12, 13, 14, 15, 16, 17, 18, 19, 20, or more. In some embodiments, it is one. In some embodiments, it is two or more. In some embodiments, it is about 3. In some embodiments, it is about 4. In some embodiments, it is about 5. In some embodiments, it is about 6. In some embodiments, it is about 7. In some embodiments, it is about 8. In some embodiments, it is about 9. In some embodiments, it is about 10. In some embodiments, it is about 10 or more.

In some embodiments, a common amino acid sequence comprises 1-1000, 1-500, 1-400, 1-300, 1-200, 1-100, 1-50, 10-1000, 10-500, 10-400, 10-300, 10-200, 10-100, 10-50, 20-1000, 20-500, 20-400, 20-300, 20-200, 20-100, 20-50, 50-1000, 50-500, 50-400, 50-300, 50-200, 50-100, 1, 2, 3, 4, 5, 6, 7, 8, 9, 10, 11, 12, 13, 14, 15, 16, 17, 18, 19, 20, 25, 30, 40, 50, 60, 70, 80, 90, 100, 110, 120, 130, 140, 150, 200, 250, 300, 400, 500, 600 or more amino acid residues. In some embodiments, a length is at least 5 amino acid residues. In some embodiments, a length is at least 10 amino acid residues. In some embodiments, a length is at least 50 amino acid residues. In some embodiments, a length is at least 100 amino acid residues. In some embodiments, a length is at least 150 amino acid residues. In some embodiments, a length is at least 200 amino acid residues. In some embodiments, a length is at least 300 amino acid residues. In some embodiments, a length is at least 400 amino acid residues. In some embodiments, a length is at least 500 amino acid residues. In some embodiments, a length is at least 600 amino acid residues.

In some embodiments, a common amino acid sequence is at least 10%-100%, 50%-100%, 50%, 55%, 60%, 65%, 70%, 75%, 80%, 85%, 90%, 91%, 92%, 93%, 94%, 95%, 96%, 97%, 98% or 99% of an amino acid sequence of a target agent moiety, a protein agent moiety, an antibody agent moiety, etc. In some embodiments, it is 100%.

In some embodiments, protein agent moieties share a high percentage of amino acid sequence homology. In some embodiments, it is 50%-100%. In some embodiments, it is 50%. In some embodiments, it is 60%. In some embodiments, it is 70%. In some embodiments, it is 80%. In some embodiments, it is 90%. In some embodiments, it is 91%. In some embodiments, it is 50%. In some embodiments, it is 92%. In some embodiments, it is 93%. In some embodiments, it is 94%. In some embodiments, it is 95%. In some embodiments, it is 96%. In some embodiments, it is 97%. In some embodiments, it is 98%. In some embodiments, it is 99%. In some embodiments, it is 100%. In some embodiments, it is at least 50%. In some embodiments, it is at least 60%. In some embodiments, it is at least 70%. In some embodiments, it is at least 80%. In some embodiments, it is at least 90%. In some embodiments, it is at least 91%. In some embodiments, it is at least 50%. In some embodiments, it is at least 92%. In some embodiments, it is at least 93%. In some embodiments, it is at least 94%. In some embodiments, it is at least 95%. In some embodiments, it is at least 96%. In some embodiments, it is at least 97%. In some embodiments, it is at least 98%. In some embodiments, it is at least 99%.

In some embodiments, a protein agent moiety or an antibody agent moiety is or comprises a protein complex. In some embodiments, at least one or each individual chain shares a common amino acid sequence and/or has a homology as described herein.

In some embodiments, agents of a plurality share a common moiety of interest. In some embodiments, each agent of a plurality is independently an agent of formula P-1 or P-II, or a salt thereof. In some embodiments, each agent of a plurality is independently an agent of formula P-1 or P-II, or a salt thereof, wherein RBM is the same for each agent of the plurality. In some embodiments, agents of a plurality are products of methods described herein. In some embodiments, compositions comprising agents of a plurality are products of methods described herein.

In some embodiments, a modification is or comprises a moiety of interest and optionally a linker. In some embodiments, a modification is or comprises -L^{PM}-RBM.

In some embodiments, agents of the plurality share a common modification independently at at least one location. In some embodiments, a modification is or comprises a moiety of interest and optionally a linker connecting the moiety of interest. As described herein, each location independently has its common modification. In some embodiments, common modifications at two or more or all locations comprise a common moiety of interest. In some embodiments, common modifications are the same. In some embodiments, agents of the plurality share a common modification at each location which has a modification that is or comprises a moiety of interest and optionally a linker. In some embodiments, agents of the plurality share a common modification at each location which has a modification that is or comprises -L^{P}M-RBM.

In some embodiments, protein agents (e.g., antibody agents) share a common modification at least one amino acid residue. In some embodiments, agents of the plurality share a common modification at each location which has a modification that is or comprises a moiety of interest and optionally a linker. In some embodiments, agents of the plurality share a common modification at each location which has a modification that is or comprises -L^{P}M-RBM.

In some embodiments, a location is selected from K246, K248, K288, K290, K317 of antibody agents and locations corresponding thereto. In some embodiments, a location is selected from K246 and K248, and locations corresponding thereto. In some embodiments, a location is selected from K288 and K290, and locations corresponding thereto. In some embodiments, a location is K246 or a location corresponding thereto. In some embodiments, a location is K248 or a location corresponding thereto. In some embodiments, a location is K288 or a location corresponding thereto. In some embodiments, a location is K290 or a location corresponding thereto. In some embodiments, a location is K317 or a location corresponding thereto. In some embodiments, a location is K185 of light chain or a location corresponding thereto. In some embodiments, a location is K187 of light chain or a location corresponding thereto. In some embodiments, a location is K133 of heavy chain or a location corresponding thereto. In some embodiments, a location is K246 or K248 of heavy chain or a location corresponding thereto. In some embodiments, a location is K414 of heavy chain or a location corresponding thereto.

In some embodiments, about 1%-100% of all agents that comprise a target agent moiety and a moiety of interest are agents of the plurality. In some embodiments, about 1%-100% of all agents that comprise a protein agent moiety that comprise the common amino acid sequence and a moiety of interest are agents of the plurality. In some embodiments, about 1%-100% of all agents that comprise an antibody agent moiety that comprise the common amino acid sequence or can bind to the common antigen and a moiety of interest are agents of the plurality. In some embodiments, about 1%-100% of all agents that comprise a target agent moiety are agents of the plurality. In some embodiments, about 1%-100% of all agents that comprise a protein agent moiety that comprise the common amino acid sequence are agents of the plurality. In some embodiments, about 1%-100% of all agents that comprise an antibody agent moiety that comprise the common amino acid sequence or can bind to the common antigen are agents of the plurality. In some embodiments, it is 50%-100%. In some embodiments, it is 50%. In some embodiments, it is 60%. In some embodiments, it is 70%. In some embodiments, it is 80%. In some embodiments, it is 90%. In some embodiments, it is 91%. In some embodiments, it is 50%. In some embodiments, it is 92%. In some embodiments, it is 93%. In some embodiments, it is 94%. In some embodiments, it is 95%. In some embodiments, it is 96%. In some embodiments, it is 97%. In some embodiments, it is 98%. In some embodiments, it is 99%. In some embodiments, it is 100%. In some embodiments, it is at least 50%. In some embodiments, it is at least 60%. In some embodiments, it is at least 70%. In some embodiments, it is at least 80%. In some embodiments, it is at least 90%. In some embodiments, it is at least 91%. In some embodiments, it is at least 50%. In some embodiments, it is at least 92%. In some embodiments, it is at least 93%. In some embodiments, it is at least 94%. In some embodiments, it is at least 95%. In some embodiments, it is at least 96%. In some embodiments, it is at least 97%. In some embodiments, it is at least 98%. In some embodiments, it is at least 99%.

In some embodiments, provided agents, compounds, etc., e.g., those of formula R—I, P—I, P-II, etc. and salts thereof have high purity. In some embodiments, it is 50%-100%. In some embodiments, it is 50%. In some embodiments, it is 60%. In some embodiments, it is 70%. In some embodiments, it is 80%. In some embodiments, it is 90%. In some embodiments, it is 91%. In some embodiments, it is 50%. In some embodiments, it is 92%. In some embodiments, it is 93%. In some embodiments, it is 94%. In some embodiments, it is 95%. In some embodiments, it is 96%. In some embodiments, it is 97%. In some embodiments, it is 98%. In some embodiments, it is 99%. In some embodiments, it is 100%. In some embodiments, it is at least 50%. In some embodiments, it is at least 60%. In some embodiments, it is at least 70%. In some embodiments, it is at least 80%. In some embodiments, it is at least 90%. In some embodiments, it is at least 91%. In some embodiments, it is at least 50%. In some embodiments, it is at least 92%. In some embodiments, it is at least 93%. In some embodiments, it is at least 94%. In some embodiments, it is at least 95%. In some embodiments, it is at least 96%. In some embodiments, it is at least 97%. In some embodiments, it is at least 98%. In some embodiments, it is at least 99%.

In some embodiments, the present disclosure provides product agent compositions comprising product agents (e.g., agents of formula P-1 or P-II, or a salt thereof). In some embodiments, a product agent composition (e.g., a formed agent composition from certain methods) comprises a product agent comprising a target agent moiety and a moiety of interest and optionally a linker (e.g., an agent of formula P-1 or P-II, or a salt thereof), a released target binding moiety (e.g., a compound comprising R^{LG}-(L^{LG1})₀₋₁-(L^{LG2})₀₋₁-(L^{LG3})₀₋₁-(L^{LG4})₀₋₁-) or a compound comprising a released target binding moiety (e.g., a compound having the structure of R^{LG}-(L^{LG1})₀₋₁-(L^{LG2})₀₋₁-(L^{LG3})₀₋₁-(L^{LG4})₀₋₁-H or a salt thereof), and a reaction partner (e.g., a compound of formula R—I or a salt thereof). In some embodiments, released target binding moieties may bind to target agent moieties in target agents and/or formed product agents. Various technologies are available to separate released target binding moieties from target agent moieties in accordance with the present disclosure, for example, in some embodiments, contacting a composition with a composition comprising glycine at certain pH.

### GENERAL METHODS, REAGENTS AND CONDITIONS

Various technologies may be utilized to provide compounds and agents herein in accordance with the present disclosure.

In some embodiments, where a particular protecting group (“PG”), leaving group (“LG”), or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

In some embodiments, leaving groups include but are not limited to, halogens (e.g. fluoride, chloride, bromide, iodide), sulfonates (e.g. mesylate, tosylate, benzenesulfonate, brosylate, nosylate, triflate), diazonium, and the like.

In some embodiments, an oxygen protecting group includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

One of skill in the art will appreciate that compounds/agents may contain one or more stereocenters, and may be present as a racemic or diastereomeric mixture. One of skill in the art will also appreciate that there are many methods known in the art for the separation of isomers to obtain stereoenriched or stereopure isomers of those compounds, including but not limited to HPLC, chiral HPLC, fractional crystallization of diastereomeric salts, kinetic enzymatic resolution (e.g. by fungal-, bacterial-, or animal-derived lipases or esterases), and formation of covalent diastereomeric derivatives using an enantioenriched reagent.

One of skill in the art will appreciate that various functional groups present in compounds of the present disclosure such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. “March's Advanced Organic Chemistry”, 5^{th} Ed., Ed.: Smith, M. B. and March, J., John Wiley & Sons, New York: 2001, the entirety of which is incorporated herein by reference. Such interconversions may require one or more of the aforementioned techniques, and certain methods for synthesizing compounds of the present disclosure are described below in the Exemplification.

### Uses, Formulations and Administration

Compounds, agents, compositions, etc. of the present disclosure may be provided as in various forms according to desired uses. In some embodiments, they are provided as pharmaceutical compositions. As appreciated by those skilled in the art, in many instances, pharmaceutical compositions comprise controlled amounts and are manufactured for administration to subjects such as human patients. In some embodiments, the present disclosure provides a composition comprising a compound, an agent, and/or a composition described herein or a pharmaceutically acceptable derivative thereof and a pharmaceutically acceptable carrier. In some embodiments, the present disclosure provides a pharmaceutical composition comprising a compound, agent or composition of the present disclosure and a pharmaceutically acceptable carrier. In some embodiments, the present disclosure provides a pharmaceutical composition comprising a therapeutically effective amount of a compound, an agent or a composition of the present disclosure and a pharmaceutically acceptable carrier. In some embodiments, a pharmaceutical composition is packaged for storage, transportation, administration, etc. In some embodiments, a pharmaceutical composition does not contain a significant amount of organic solvents (e.g., total amount of organic solvents no more than 50%, 40%, 30%, 25%, 20%, 15%, 10%, 5%, 4%, 3%, 2%, or 1% of weight and/or volume of a pharmaceutical composition).

In some embodiments, a pharmaceutically acceptable carrier is or comprises a non-toxic carrier, adjuvant, or vehicle that does not destroy the pharmacological activity of the compound with which it is formulated. Pharmaceutically acceptable carriers, adjuvants or vehicles that may be include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In some embodiments, a pharmaceutically acceptable derivative is a non-toxic salt, ester, salt of an ester or other derivative of a compound that, upon administration to a recipient, is capable of providing, either directly or indirectly, a compound or an active metabolite or residue thereof.

Compositions may be administered orally, parenterally, by inhalation spray, topically, rectally, nasally, buccally, vaginally or via an implanted reservoir. In some embodiments, parenteral administration includes subcutaneous, intravenous, intramuscular, intra-articular, intra-synovial, intrasternal, intrathecal, intrahepatic, intralesional and intracranial injection or infusion techniques. In some embodiments, compositions are administered orally, intraperitoneally or intravenously. Sterile injectable forms of compositions may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

In some embodiments, a bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

In some embodiments, pharmaceutically acceptable compositions may be administered in the form of suppositories for rectal administration. In some embodiments, these can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

In some embodiments, pharmaceutically acceptable compositions may be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, pharmaceutically acceptable compositions may be formulated in a suitable ointment containing the active component suspended or dissolved in one or more carriers.

Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride.

Alternatively, for ophthalmic uses, the pharmaceutically acceptable compositions may be formulated in an ointment such as petrolatum.

Pharmaceutically acceptable compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, pharmaceutically acceptable compositions are formulated for oral administration. Such formulations may be administered with or without food. In some embodiments, pharmaceutically acceptable compositions are administered without food. In other embodiments, pharmaceutically acceptable compositions are administered with food.

Amounts of compounds that may be combined with the carrier materials to produce a composition in a single dosage form will vary depending upon the host treated, the particular mode of administration. In some embodiments, provided compositions are formulated so that a dosage of between 0.01-100 mg/kg body weight/day of the modulator can be administered to a patient receiving these compositions.

In some embodiments, the present disclosure is directed to compositions that include therapy enhancer agents containing moieties of interest conjugated to target agent moieties at specific locations.

In an embodiment, provided is a composition including:
a first compound having the structure of formula PN-L:

P^{IG}—N-L^{PM}-RBM PN—I
wherein:
P^{IG}—N is a human plasma immunoglobulin moiety including a lysine residue;
L^{PM} is a linker; and
RBM is an immune cell surface receptor binding moiety capable of modulating the immune cell surface receptor; and
a second compound having the structure of formula LG-L:

LG^{IG}-OH LG-I
wherein LG^{IG} is a group including a target binding moiety that binds to a human plasma immunoglobulin with site-directed specificity.

In another embodiment, the composition further includes:
a third compound having the formula R-L:

LG^{IG}-RG-L^{RM}-RBM R—I
LG^{IG} is a group including a target binding moiety that binds to a target agent with site-directed specificity, which is identical to LG in formula (LG-1);
RG is a reactive group;
L^{RM} is a linker, which is identical to in formula (P-II); and
RBM is an immune cell surface receptor binding moiety capable of modulating the immune cell surface receptor.
a fourth compound having the formula R—III:

HO-RG-L^{RM}-RBM R—III
or a combination thereof.

In an embodiment, provided is a composition including:
a first compound having the structure of formula PN-II:

P^{HG}—N-L^{PM}-RBM PN-II
wherein:
P^{HG}—N is an hyperimmune globulin moiety including a lysine residue;
L^{PM} is a linker; and
RBM is an immune cell surface receptor binding moiety capable of modulating the cell surface receptor; and
a second compound having the structure of formula LG-II:

LG^{HG}-OH LG-II
wherein LGH^{G} is a group including a target binding moiety that binds to a hyperimmune globulin with site-directed specificity.

In another embodiment, the composition further includes:
a third compound having the formula R-II:

LG^{HG}-RG-L^{RM}-RBM R-II
LG^{HG} is a group including a target binding moiety that binds to a hyperimmune globulin with site-directed specificity;
RG is a reactive group;
L^{RM} is a linker; and
RBM is an immune cell surface receptor binding moiety capable of modulating the immune cell surface receptor.
a fourth compound having the formula R—III:

HO-RG-L^{RM}-RBM R—III
or a combination thereof.

In some embodiment, the compositions may include the first and second compounds in equimolar amount. In some embodiments, the amount of the second compound may be 50 mole percent (mole %) or less based on the total number of moles of the first and second compounds in the composition. In some embodiments, the amount of the second compound may be 50 mole % or less, 45 mole % or less, 40 mole % or less, 35 mole % or less, 30 mole % or less, 25 mole % or less, 20 mole % or less, 15 mole % or less, 10 mole % or less, or 5 mole % or less based on the total number of moles of the first and second compounds in the composition. In some embodiments, the amount of the second compound may be 5% or less, 4% or less, 3% or less, 2% or less, or 1% or less based on the total number of moles of the first and second compounds in the composition. In some embodiments, the amount of the second compound may be 1.0% or less, 0.9% or less, 0.8% or less, 0.7% or less, 0.6% or less, 0.5% or less, 0.4% or less, 0.3% or less, 0.2% or less, 0.1% or less based on the total number of moles of the first and second compounds in the composition. In some embodiments, the amount of the second compound may be 0.10% or less, 0.09% or less, 0.08% or less, 0.07% or less, 0.06% or less, 0.05% or less, 0.04% or less, 0.03% or less, 0.02% or less, 0.01% or less based on the total number of moles of the first and second compounds in the composition. In some embodiments, the amount of the second compound may be 0.010% or less, 0.009% or less, 0.008% or less, 0.007% or less, 0.006% or less, 0.005% or less, 0.004% or less, 0.003% or less, 0.002% or less, 0.001% or less based on the total number of moles of the first and second compounds in the composition. In some embodiments, the amount of the second compound may be 0.0010% or less, 0.0009% or less, 0.0008% or less, 0.0007% or less, 0.0006% or less, 0.0005% or less, 0.0004% or less, 0.0003% or less, 0.0002% or less, 0.0001% or less based on the total number of moles of the first and second compounds in the composition. In some embodiments, the amount of the second compound may be 0.00010% or less, 0.00009% or less, 0.00008% or less, 0.00007% or less, 0.00006% or less, 0.00005% or less, 0.00004% or less, 0.00003% or less, 0.00002% or less, 0.00001% or less based on the total number of moles of the first and second compounds in the composition. In some embodiments, the amount of the second compound may be 0.000010% or less, 0.000009% or less, 0.000008% or less, 0.000007% or less, 0.000006% or less, 0.000005% or less, 0.000004% or less, 0.000003% or less, 0.000002% or less, 0.000001% or less based on the total number of moles of the first and second compounds in the composition.

In some embodiment, the compositions may further include a third compound, a fourth compound, or a combination thereof. In some embodiments, the amount of the third compound, the fourth compound, or the combination thereof may be 5% or less, 4% or less, 3% or less, 2% or less, or 1% or less based on the number of moles of the first compound in the composition. In some embodiments, the amount of the third compound, the fourth compound, or the combination thereof may be 1.0% or less, 0.9% or less, 0.8% or less, 0.7% or less, 0.6% or less, 0.5% or less, 0.4% or less, 0.3% or less, 0.2% or less, 0.1% or less based on the number of moles of the first compound in the composition. In some embodiments, the amount of the third compound, the fourth compound, or the combination thereof may be 0.10% or less, 0.09% or less, 0.08% or less, 0.07% or less, 0.06% or less, 0.05% or less, 0.04% or less, 0.03% or less, 0.02% or less, 0.01% or less based on the number of moles of the first compound in the composition. In some embodiments, the amount of the third compound, the fourth compound, or the combination thereof may be 0.010% or less, 0.009% or less, 0.008% or less, 0.007% or less, 0.006% or less, 0.005% or less, 0.004% or less, 0.003% or less, 0.002% or less, 0.001% or less based on the number of moles of the first compound in the composition. In some embodiments, the amount of the third compound, the fourth compound, or the combination thereof may be 0.0010% or less, 0.0009% or less, 0.0008% or less, 0.0007% or less, 0.0006% or less, 0.0005% or less, 0.0004% or less, 0.0003% or less, 0.0002% or less, 0.0001% or less based on the number of moles of the first compound in the composition. In some embodiments, the amount of the third compound, the fourth compound, or the combination thereof may be 0.00010% or less, 0.00009% or less, 0.00008% or less, 0.00007% or less, 0.00006% or less, 0.00005% or less, 0.00004% or less, 0.00003% or less, 0.00002% or less, 0.00001% or less based on the number of moles of the first compound in the composition. In some embodiments, the amount of the third compound, the fourth compound, or the combination thereof may be 0.000010% or less, 0.000009% or less, 0.000008% or less, 0.000007% or less, 0.000006% or less, 0.000005% or less, 0.000004% or less, 0.000003% or less, 0.000002% or less, 0.000001% or less based on the number of moles of the first compound in the composition.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

Technologies (e.g., compounds, agents, compositions) of the present disclosure can be utilized for various purposes, e.g., detection, diagnosis, therapy, etc. In some embodiments, provided technologies are useful for treating conditions, disorders or diseases, e.g., various cancers. In some embodiments, provided technologies comprise target binding moieties, e.g., antibody agent moieties, that can bind antigens of cancer cells. In some embodiments, a target binding moiety is an antibody agent moiety. In some embodiments, an antibody agent is a therapeutic agent. Among other things, various antibody agents, including many developed and/or approved (e.g., by FDA, EMA, etc.) as therapeutics can be utilized in accordance with the present disclosure to provide therapeutics for various diseases.

Throughout this application, various publications are referenced by author name and date, or by patent number or patent publication number. The disclosures of these publications are hereby incorporated in their entireties by reference into this application in order to more fully describe the state of the art as known to those skilled therein as of the date of the disclosure described and claimed herein. However, the citation of a reference herein should not be construed as an acknowledgement that such reference is prior art to the present invention.

Those skilled in the art will recognize, or be able to ascertain using no more than routine experimentation, numerous equivalents to the specific procedures described herein. Such equivalents are considered to be within the scope of this disclosure and are covered by the following claims. For example, pharmaceutically acceptable salts other than those specifically disclosed in the description and Examples herein can be employed. Furthermore, it is intended that specific items within lists of items, or subset groups of items within larger groups of items, can be combined with other specific items, subset groups of items or larger groups of items whether or not there is a specific disclosure herein identifying such a combination.

1. An agent comprising:
a human plasma immunoglobulin moiety,
an immune cell surface receptor binding moiety capable of modulating the immune cell surface receptor, and
optionally a linker moiety linking the human plasma immunoglobulin moiety and the immune cell surface receptor binding moiety.

2. The agent of claim 1, wherein the immune cell surface receptor is an Fc receptor.

3. The agent of claim 1, wherein the immune cell surface receptor is CD16A (FcγRIIIa) or CD32B (FcγRIIb).

4. The agent of claim 1, wherein the immune cell surface receptor is NKG2D.

5. The agent of any one of claims 1 to 4, wherein the agent has the structure of formula M-1: 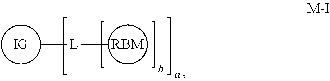
or a pharmaceutically acceptable salt thereof, wherein:
a is 1 or 2;
b is 1, 2, or 3;
IG is a human plasma immunoglobulin moiety;
each L is a linker moiety; and
RBM is an immune cell surface receptor binding moiety capable of modulating the immune cell surface receptor,
wherein the human plasma immunoglobulin moiety comprises an immunoglobulin or a fragment thereof from an IVIG preparation.

6. The agent of any one of claims 1 to 5, wherein the immunoglobulin moiety comprises IgG1 or a fragment thereof, IgG2 or a fragment thereof, or IgG4 or a fragment thereof.

7. The agent of any one of claims 1 to 6, wherein the immunoglobulin moiety comprises IgG1 or a fragment thereof is linked to the linker L, at an amino acid residue selected from K246 and K248 of an IgG1 heavy chain and amino acid residues corresponding thereto; or
the immunoglobulin moiety comprises IgG2 or a fragment thereof IgG2 or a fragment thereof is linked to the linker, at an amino acid residue selected from K251 and K253 of an IgG2 heavy chain and amino acid residues corresponding thereto; or
the immunoglobulin moiety comprises IgG4 or a fragment thereof is linked to the linker, at an amino acid residue selected from K239 and K241 of an IgG4 heavy chain and amino acid residues corresponding thereto.

8. The agent of any one of claims 5 to 7, wherein
L is a covalent bond, or a bivalent or polyvalent optionally substituted, linear or branched C₁₋₁₀₀ group comprising one or more aliphatic, aryl, heteroaromatic having 1-20 heteroatoms, or any combinations thereof, wherein one or more methylene units of the group are optionally and independently replaced with C₁₋₆ alkylene, C₁₋₆ alkenylene, —C≡C—, -Cy-, —C(R′)₂—, —O—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or -[(—O—C(R′)₂—C(R′)₂-)ₙ]-, wherein n is 1-20;
-Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
each R′ is independently —R, —C(O)R, —CO₂R, or —SO₂R; and
each R is independently —H, or an optionally substituted.

9. The agent of any one of claims 5 to 8, wherein the linker comprises one or more —[(CH₂)ₙ-0]m-, wherein each n is independently 1-20, and m is 1-100.

10. A method of treating an acute or chronic inflammatory disorder in a patient in need thereof, comprising administering to the patient a pharmaceutically effective amount of the agent of one of claims 1 to 9.

11. A method of treating an acute or chronic autoimmune disorder in a patient in need thereof, comprising administering to the patient a pharmaceutically effective amount of the agent of one of claims 1 to 9.

12. A method of treating cancer in a patient in need thereof, comprising administering to the patient a pharmaceutically effective amount of the agent of one of claims 1 to 9.

13. An agent comprising:
an hyperimmune globulin moiety,
an immune cell surface receptor binding moiety capable of modulating the immune cell surface receptor, and
optionally a linker moiety linking the hyperimmune globulin moiety and the immune cell surface receptor binding moiety.

14. The agent of claim 13, wherein the immune cell surface receptor is CD16A, CD32B, NKG2D, or DC-SIGN.

15. The agent of claim 13, wherein the agent has the structure of formula M-II: 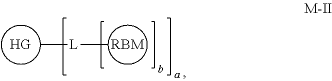
or a pharmaceutically acceptable salt thereof, wherein:
a is 1 or 2;
b is 1, 2, or 3;
HG is a hyperimmune globulin moiety;
each L is a linker moiety; and
RBM is an immune cell surface receptor binding moiety capable of modulating the immune cell surface receptor,
wherein the immune cell surface receptor is DC-SIGN.

16. The agent of claim 14 or 15, wherein the hyperimmune globulin moiety comprises HIgG1 or a fragment thereof, HIgG2 or a fragment thereof, or HIgG4 or a fragment thereof.

17. The agent of any one of claims 14 to 16, wherein the hyperimmune globulin moiety comprises HIgG1 or a fragment thereof that is linked to the linker L, at an amino acid residue selected from K246 and K248 of an HIgG1 heavy chain and amino acid residues corresponding thereto; or
the hyperimmune globulin moiety comprises HIgG2 or a fragment thereof that is linked to the linker, at an amino acid residue selected from K251 and K253 of an HIgG2 heavy chain and amino acid residues corresponding thereto; or
the hyperimmune globulin moiety comprises IgG4 or a fragment thereof that is linked to the linker, at an amino acid residue selected from K239 and K241 of an IgG4 heavy chain and amino acid residues corresponding thereto.

18. The agent of any one of claims 15 to 17, wherein
L is a covalent bond, or a bivalent or polyvalent optionally substituted, linear or branched C₁₋₁₀₀ group comprising one or more aliphatic, aryl, heteroaromatic having 1-20 heteroatoms, or any combinations thereof, wherein one or more methylene units of the group are optionally and independently replaced with C₁₋₆ alkylene, C₁₋₆ alkenylene, —C—C—, -Cy-, —C(R′)₂—, —O—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or -[(—O—C(R′)₂—C(R′)₂-)ₙ]-, wherein n is 1-20;
-Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
each R′ is independently —R, —C(O)R, —CO₂R, or —SO₂R; and
each R is independently —H, or an optionally substituted.

19. The agent of any one of claims 15 to 18, wherein the linker comprises one or more —[(CH₂)ₙ—O]ₘ—, wherein each n is independently 1-20, and m is 1-100.

20. A method of treating an infectious disease in a patient in need thereof, comprising administering to the patient a pharmaceutically effective amount of the agent of one of claims 14 to 19.

21. The method of claim 20, wherein the infectious disease is a viral infection.

22. The method of claim 21, wherein the viral infection is influenza.

23. A method of treating an acute or chronic inflammatory disorder in a patient in need thereof, comprising administering to the patient a pharmaceutically effective amount of the agent of one of claims 14 to 19.

24. A method of treating an acute or chronic autoimmune disorder in a patient in need thereof, comprising administering to the patient a pharmaceutically effective amount of the agent of one of claims 14 to 19.

25. A method of treating cancer in a patient in need thereof, comprising administering to the patient a pharmaceutically effective amount of the agent of one of claims 14 to 19.

26. An agent, wherein the agent has the structure of formula R-L:
LG^{IG}-RG-L^{RM}-RBM, R—I
or a salt thereof, wherein:
LG^{IG} is R^{LG}-L^{LG};
R^{LG} is a moiety capable of binding to a human plasma immunoglobulin with site-directed specificity that is selected from 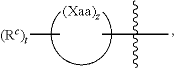
R^{c}-(Xaa)z-, IVIG, a nucleic acid moiety, and a small molecule moiety;
each Xaa is independently a residue of an amino acid or an amino acid analog;
t is 0-50;
z is 1-50;
each R^{c} is independently -L^{a}-R′;
each of a and b is independently 1-200;
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
L^{LG} is -L^{LG1}-, -L^{LG1}-L^{LG2}-, -L^{LG1}-L^{LG2}-L^{LG3}-, or -L^{LG1}-L^{LG2}-L^{LG3}-L^{LG4}-;
RG is -L^{RG1}-L^{RG2}-, -L^{LG4}-L^{RG1}-L^{RG2}-, -L^{LG3}-L^{LG4}-LRG-L^{RG2}-, -L^{LG2}-L^{LG3}-L^{LG4}-L^{RG1}-L^{RG2}-;
each of L^{LG1}, L^{LG2}, L^{LG3}, L^{LG4}, L^{RG1}, L^{RG2}, and L^{RM} is independently L;
each L is independently a covalent bond, or a bivalent optionally substituted, linear or branched C₁₋₁₀₀ group comprising one or more aliphatic moieties, aryl moieties, heteroaliphatic moieties each independently having 1-20 heteroatoms, heteroaromatic moieties each independently having 1-20 heteroatoms, or any combinations of any one or more of such moieties, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or -[(—O—C(R′)₂—C(R′)₂-)ₙ]-, wherein n is 1-20;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms; and
RBM is a cell surface receptor binding moiety capable of modulating the cell surface receptor,
wherein the cell surface receptor is CD16, CD32, or NKG2.

27. The agent of claim 26, wherein LG^{IG} comprises or has the structure of
(i) DCAWHLGELVWCT or a salt form thereof, wherein the two C residues are linked by a —S—S—;
(ii) DCAWHLGELVWCT or a salt form thereof, wherein the N-terminus is capped with R—C(O)—; or
(iii) DCAWHLGELVWCT or a salt form thereof, wherein the N-terminus is capped with R—C(O)—, wherein R is methyl; or
(iv) DCAWHLGELVWCT or a salt form thereof, wherein the antibody binding moiety is connected to the rest of a molecule through its C-terminus.

28. The agent of claim 26, wherein LG^{IG} comprises or has the structure selected from A-1 to A-50, or a salt form thereof.

29. The agent of any one of claims 26-28, wherein each L is independently a covalent bond, or a bivalent optionally substituted, linear or branched aliphatic group or heteroaliphatic group having 1-10 heteroatoms, wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or -[(—O—C(R′)₂—C(R′)₂-)ₙ]-, wherein n is 1-20.

30. The agent of any one of claims 26-29, wherein
LG^{IG} is R^{LG}-L^{LG}-, wherein R^{LG} is or comprises a target binding moiety and L^{LG} is L^{LG1}, L^{LG1}-L^{LG2}-, L^{LG1}-L^{LG2}-L^{LG3}-, or L^{LG1}-L^{LG2}-L^{LG3}-L^{LG4}-, wherein each L^{LG} is independently chosen from L.

31. The agent of any one of claims 26-30, wherein RG is or comprises -L^{LG2}-L^{LG3}-L^{LG4}-L^{RG1}-L^{RG1}-, -L^{LG2}-L^{LG3}-L^{LG4}-L^{RG1}-L^{RG2}-, -L^{LG3}-L^{LG4}-L^{RG1}-L^{RG2}-L^{LG4}-L^{RG1}-L^{RG2}-, or -L^{RG1}-L^{RG2}-, wherein L^{LG1} is a covalent bond, —(CH₂CH₂O)ₙ—, or —(CH₂)ₙ—O—(CH₂CH₂O)ₙ—(CH₂)ₙ—;
L^{LG2} is or comprises a covalent bond, —NR′—, —C(O)—, —NR′C(O)—, —(CH₂)ₙ—OC(O)N(R′)—, —CH₂N(CH₂CH₂CH₂S(O)₂OH)—C(O)—, —C(O)—NHCH₂—, —C(O)O—CH₂—, or —NH—C(O)O—CH₂—;
R′ is H or C₁-C₆alkyl;
L^{LG3} is optionally bonded to —C(O)— and L^{LG3} a covalent bond or a substituted phenyl ring, substituted with one or more substituents, and one or more substituents are independently an electron-withdrawing group;
L^{LG3} is 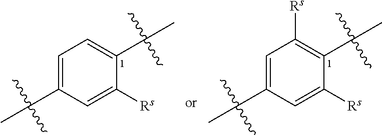
where R^{s} is F or NO₂;
L^{LG4} is a covalent bond, —O—, —NR′—;
L^{RG1} is a covalent bond, —S(O)₂— or —C(O)—;
L^{RG2} is or comprises —C(O)—, —C(O)O—, —C(O)N(R′)—, —S(O)—, —S(O)₂—, —P(O)(OR′)—, —P(O)(SR′)—, or —P(O)(N(R′)₂)—;
wherein each n is independently 1-10, and each —CH₂— is independently optionally substituted.

32. The agent of any one of claims 26-31, wherein L^{LG3} is 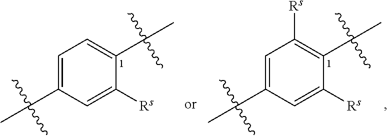
and wherein each R⁵ is F or NO₂.

33. The agent of any one of claims 26-32, wherein L^{RG2} is or comprises -L^{RG3}-C(═CR^{RG1}R^{RG2})—CR^{RG3}R^{RG4}— or -L^{RG3}-C(═CHR^{RG2})—CHR^{RG4}—, wherein each of R^{RG1}, R^{RG2}, R^{RG3} and R^{RG4} is independently -L-R′, and L^{RG3} is —C(O)—, —C(O)O—, —C(O)N(R′)—, —S(O)—, —S(O)₂—, —P(O)(OR′)—, —P(O)(SR′)—, or —P(O)(N(R′)₂)—.

34. The agent of any one of claims 26-33, wherein -L^{LG2}-L^{LG3}-L^{LG4}-L^{RG1}- is a structure selected from:

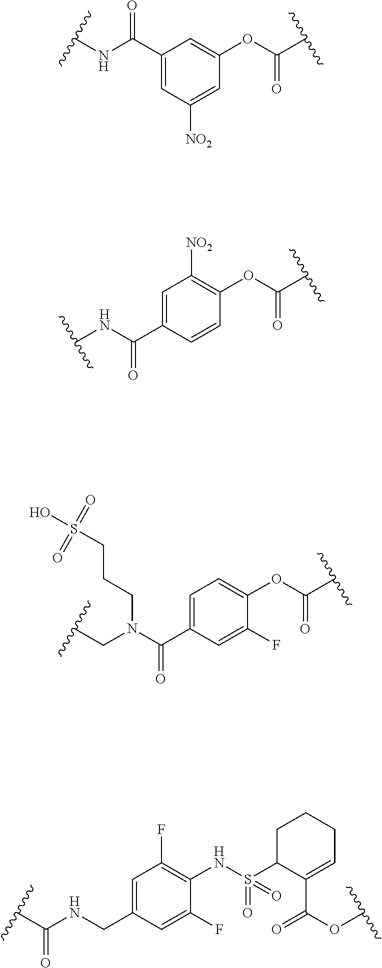 [SMILES: *NC(=O)c1cc(OC(*)=O)cc([N+](=O)[O-])c1 ; *NC(=O)c1ccc(OC(*)=O)c([N+](=O)[O-])c1 ; *CN(CCCS(=O)(=O)O)C(=O)c1ccc(OC(*)=O)c(F)c1 ; *OC(=O)C1=CCCCC1S(=O)(=O)Nc1c(F)cc(CNC(*)=O)cc1F]

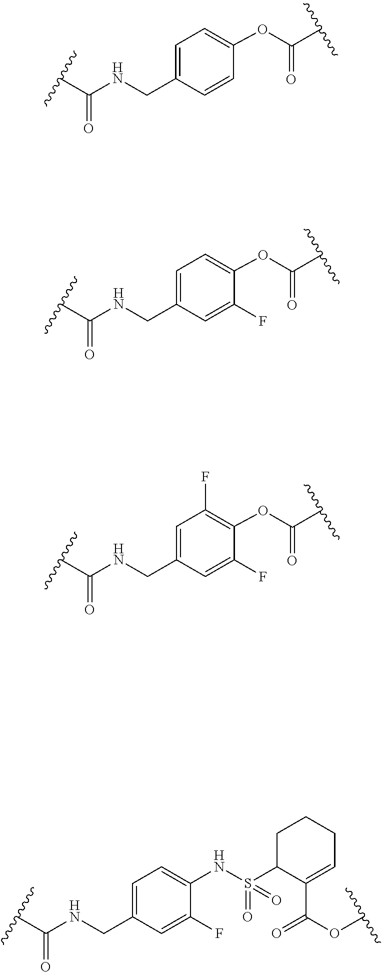 [SMILES: *C(=O)NCc1ccc(OC(*)=O)cc1 ; *C(=O)NCc1ccc(OC(*)=O)c(F)c1 ; *C(=O)NCc1cc(F)c(OC(*)=O)c(F)c1 ; *OC(=O)C1=CCCCC1S(=O)(=O)Nc1ccc(CNC(*)=O)cc1F]

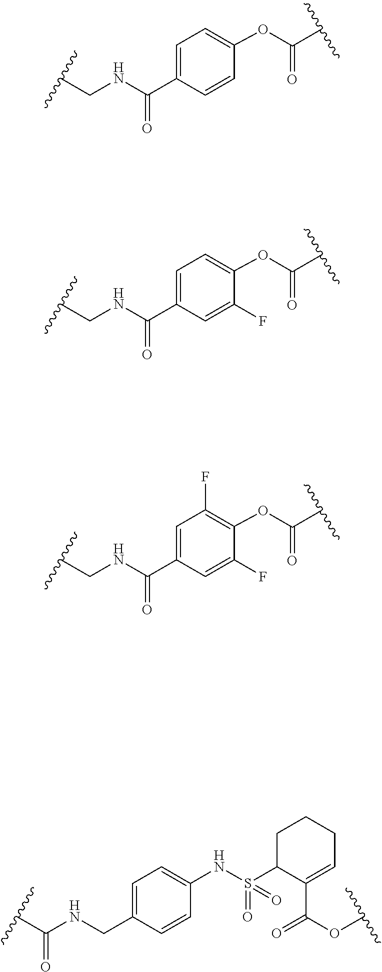 [SMILES: *CNC(=O)c1ccc(OC(*)=O)cc1 ; *CNC(=O)c1ccc(OC(*)=O)c(F)c1 ; *CNC(=O)c1cc(F)c(OC(*)=O)c(F)c1 ; *OC(=O)C1=CCCCC1S(=O)(=O)Nc1ccc(CNC(*)=O)cc1]

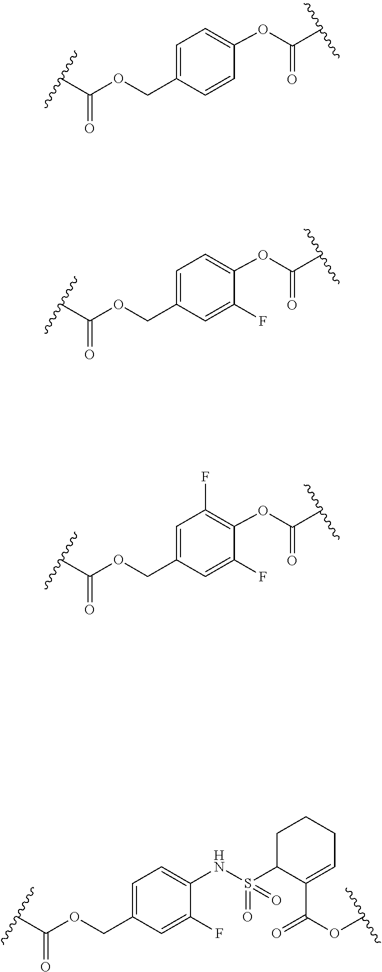 [SMILES: *C(=O)OCc1ccc(OC(*)=O)cc1 ; *C(=O)OCc1ccc(OC(*)=O)c(F)c1 ; *C(=O)OCc1cc(F)c(OC(*)=O)c(F)c1 ; *OC(=O)C1=CCCCC1S(=O)(=O)Nc1ccc(COC(*)=O)cc1F]

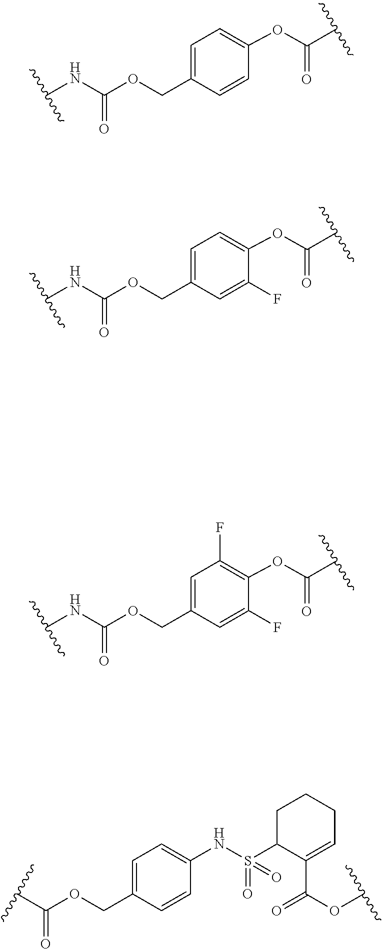 [SMILES: *NC(=O)OCc1ccc(OC(*)=O)cc1 ; *NC(=O)OCc1ccc(OC(*)=O)c(F)c1 ; *NC(=O)OCc1cc(F)c(OC(*)=O)c(F)c1 ; *OC(=O)C1=CCCCC1S(=O)(=O)Nc1ccc(COC(*)=O)cc1]

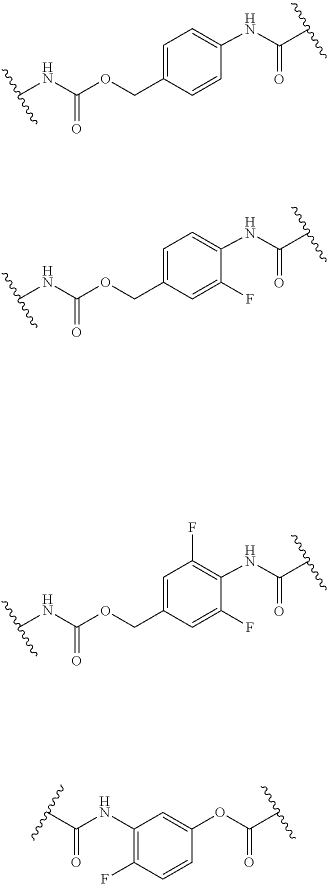 [SMILES: *NC(=O)OCc1ccc(NC(*)=O)cc1 ; *NC(=O)OCc1ccc(NC(*)=O)c(F)c1 ; *NC(=O)OCc1cc(F)c(NC(*)=O)c(F)c1 ; *C(=O)Nc1cc(OC(*)=O)ccc1F]

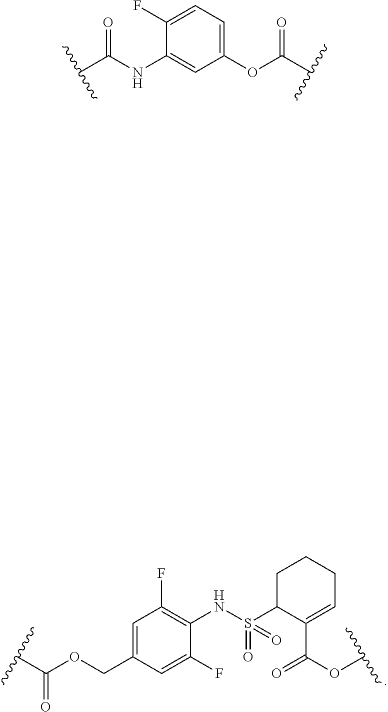 [SMILES: *C(=O)Nc1cc(OC(*)=O)ccc1F ; *OC(=O)C1=CCCCC1S(=O)(=O)Nc1c(F)cc(COC(*)=O)cc1F]

35. An agent, wherein the agent has the structure of formula R-II:
LG^{HG}-RG-L^{RM}-RBM, R-II
or a salt thereof, wherein:
LG^{HG} is R^{LG}-L^{LG};
R^{G} is a moiety capable of binding to a human plasma immunoglobulin with site-directed specificity that is selected from 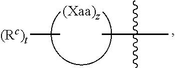
R^{c}-(Xaa)z-, IVIG, a nucleic acid moiety, and a small molecule moiety;
each Xaa is independently a residue of an amino acid or an amino acid analog;
t is 0-50;
z is 1-50;
each R^{c} is independently -L^{a}-R′;
each of a and b is independently 1-200;
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
L^{LG} is -L^{LG1}-, -L^{LG1}-L^{LG2}-, -L^{LG1}-L^{LG2}-L^{LG3}-, or -L^{LG1}-L^{LG2}-L^{LG3}-L^{LG4}-;
RG is -L^{RG1}-L^{RG2}-, -L^{LG4}-L^{RG1}-L^{RG2}-, -L^{LG3}-L^{LG4}-LRG-L^{RG2}-, -L^{LG2}-L^{LG3}-L^{LG4}-L^{RG1}-L^{RG2}-;
each of L^{LG1}, L^{LG2}, L^{LG3}, L^{LG4}, L^{RG1}, L^{RG2}, and L^{RM} is independently L;
each L is independently a covalent bond, or a bivalent optionally substituted, linear or branched C₁₋₁₀₀ group comprising one or more aliphatic moieties, aryl moieties, heteroaliphatic moieties each independently having 1-20 heteroatoms, heteroaromatic moieties each independently having 1-20 heteroatoms, or any combinations of any one or more of such moieties, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or -[(—O—C(R′)₂—C(R′)₂-)ₙ]-, wherein n is 1-20;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms; and
RBM is a cell surface receptor binding moiety capable of modulating the cell surface receptor,
wherein the cell surface receptor is DC-SIGN.

36. The agent of claim 35, wherein LG^{HG} comprises or has the structure of
(i) DCAWHLGELVWCT or a salt form thereof, wherein the two C residues are linked by a —S—S—;
(ii) DCAWHLGELVWCT or a salt form thereof, wherein the N-terminus is capped with R—C(O)—; or
(iii) DCAWHLGELVWCT or a salt form thereof, wherein the N-terminus is capped with R—C(O)—, wherein R is methyl; or
(iv) DCAWHLGELVWCT or a salt form thereof, wherein the antibody binding moiety is connected to the rest of a molecule through its C-terminus.

37. The agent of claim 36, wherein LG^{HG} comprises or has the structure selected from A-1 to A-50, or a salt form thereof.

38. The agent of any one of claims 35-37, wherein each L is independently a covalent bond, or a bivalent optionally substituted, linear or branched aliphatic group or heteroaliphatic group having 1-10 heteroatoms, wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or -[(—O—C(R′)₂—C(R′)₂-)ₙ]-, wherein n is 1-20.

39. The agent of any one of claims 35-38, wherein
LG^{HG} is R^{LG}-L^{LG}-, wherein R^{LG} is or comprises a target binding moiety and L^{LG} is L^{LG1}, L^{LG1}-L^{LG2}-, L^{LG1}-L^{LG2}-L^{LG3}-, or L^{LG1}-L^{LG2}-L^{LG3}-L^{LG4}-, wherein each L^{LG} is independently chosen from L.

40. The agent of any one of claims 35-39, wherein RG is or comprises -L^{LG2}-L^{LG3}-L^{LG4}-L^{RG1}-L^{RG1}-, -L^{LG2}-L^{LG3}-L^{LG4}-L^{RG1}-L^{RG2}-, -L^{LG3}-L^{LG4}-L^{RG1}-L^{RG2}-, L^{LG4}-L^{RG1}-L^{RG2}-, or -L^{RG1}-L^{RG2}-, wherein
L^{LG1} is a covalent bond, —(CH₂CH₂O)ₙ—, or —(CH₂)ₙ—O—(CH₂CH₂O)ₙ—(CH₂)ₙ—;
L^{LG2} is or comprises a covalent bond, —NR′—, —C(O)—, —NR′C(O)—, —(CH₂)ₙ—OC(O)N(R′)—, —CH₂N(CH₂CH₂CH₂S(O)₂OH)—C(O)—, —C(O)—NHCH₂—, —C(O)O—CH₂—, or —NH—C(O)O—CH₂—;
R′ is H or C₁-C₆alkyl;
L^{LG3} is optionally bonded to —C(O)— and L^{LG3} a covalent bond or a substituted phenyl ring, substituted with one or more substituents, and one or more substituents are independently an electron-withdrawing group;
L^{LG3} is 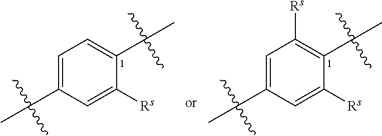
where R^{s} is F or NO₂;
L^{LG4} is a covalent bond, —O—, —NR′—;
L^{RG1} is a covalent bond, —S(O)₂— or —C(O)—;
L^{RG2} is or comprises —C(O)—, —C(O)O—, —C(O)N(R′)—, —S(O)—, —S(O)₂—, —P(O)(OR′)—, —P(O)(SR′)—, or —P(O)(N(R′)₂)—;
wherein each n is independently 1-10, and each —CH₂— is independently optionally substituted.

41. The agent of any one of claims 35-40, wherein L^{LG3} is 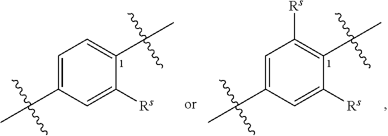
and wherein each R^{s} is F or NO₂.

42. The agent of any one of claims 35-41, wherein L^{RG2} is or comprises -L^{RG3}-C(═CR^{RG1}R^{RG2})—CR^{RG3}R^{RG4}— or -L^{RG3}-C(═CHR^{RG2})—CHR^{RG4}—, wherein each of R^{RG1}, R^{RG2}, R^{RG3} and R^{RG4} is independently -L-R′, and L^{RG3} is —C(O)—, —C(O)O—, —C(O)N(R′)—, —S(O)—, —S(O)₂—, —P(O)(OR′)—, —P(O)(SR′)—, or —P(O)(N(R′)₂)—.

43. The agent of any one of claims 35-42, wherein -L^{LG2}-L^{LG3}-L^{LG4}-L^{RG1}- is a structure selected from: 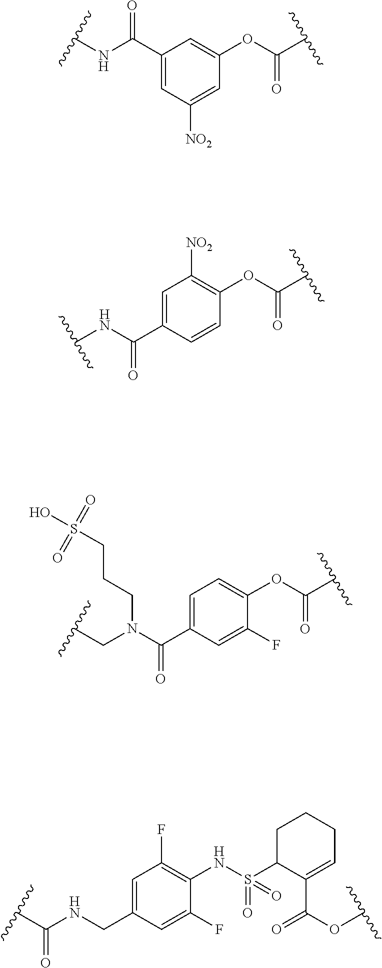 [SMILES: *NC(=O)c1cc(OC(*)=O)cc([N+](=O)[O-])c1 ; *NC(=O)c1ccc(OC(*)=O)c([N+](=O)[O-])c1 ; *CN(CCCS(=O)(=O)O)C(=O)c1ccc(OC(*)=O)c(F)c1 ; *OC(=O)C1=CCCCC1S(=O)(=O)Nc1c(F)cc(CNC(*)=O)cc1F] 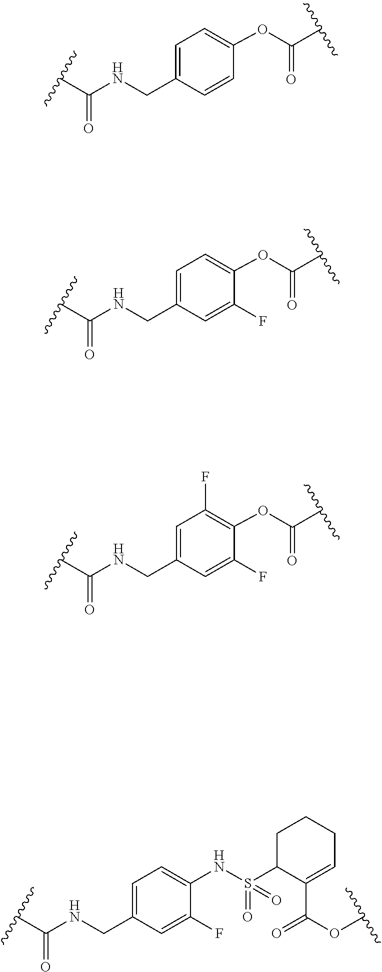 [SMILES: *C(=O)NCc1ccc(OC(*)=O)cc1 ; *C(=O)NCc1ccc(OC(*)=O)c(F)c1 ; *C(=O)NCc1cc(F)c(OC(*)=O)c(F)c1 ; *OC(=O)C1=CCCCC1S(=O)(=O)Nc1ccc(CNC(*)=O)cc1F] 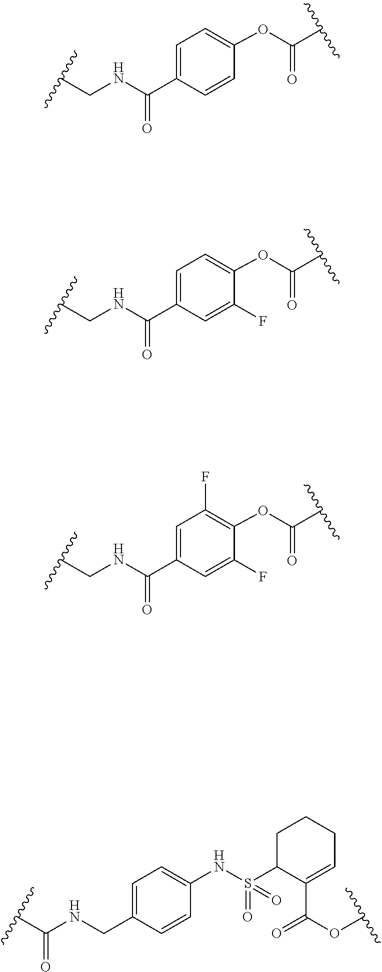 [SMILES: *CNC(=O)c1ccc(OC(*)=O)cc1 ; *CNC(=O)c1ccc(OC(*)=O)c(F)c1 ; *CNC(=O)c1cc(F)c(OC(*)=O)c(F)c1 ; *OC(=O)C1=CCCCC1S(=O)(=O)Nc1ccc(CNC(*)=O)cc1] 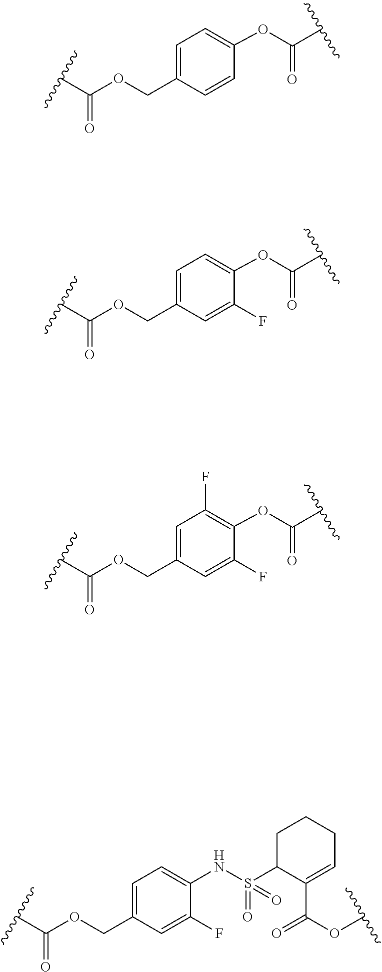 [SMILES: *C(=O)OCc1ccc(OC(*)=O)cc1 ; *C(=O)OCc1ccc(OC(*)=O)c(F)c1 ; *C(=O)OCc1cc(F)c(OC(*)=O)c(F)c1 ; *OC(=O)C1=CCCCC1S(=O)(=O)Nc1ccc(COC(*)=O)cc1F] 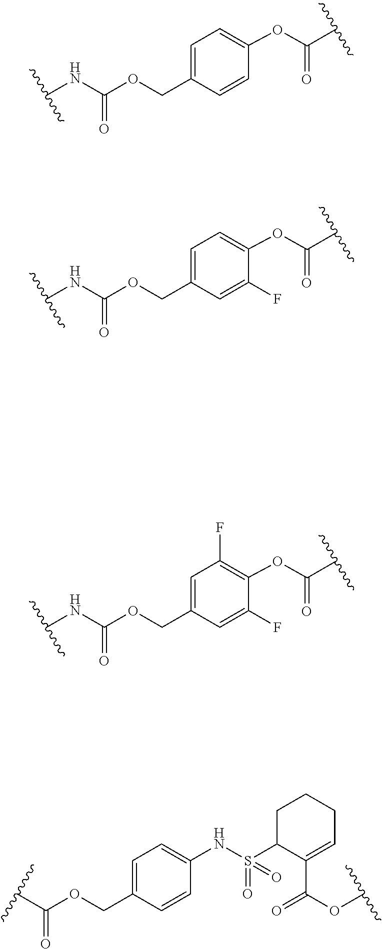 [SMILES: *NC(=O)OCc1ccc(OC(*)=O)cc1 ; *NC(=O)OCc1ccc(OC(*)=O)c(F)c1 ; *NC(=O)OCc1cc(F)c(OC(*)=O)c(F)c1 ; *OC(=O)C1=CCCCC1S(=O)(=O)Nc1ccc(COC(*)=O)cc1] 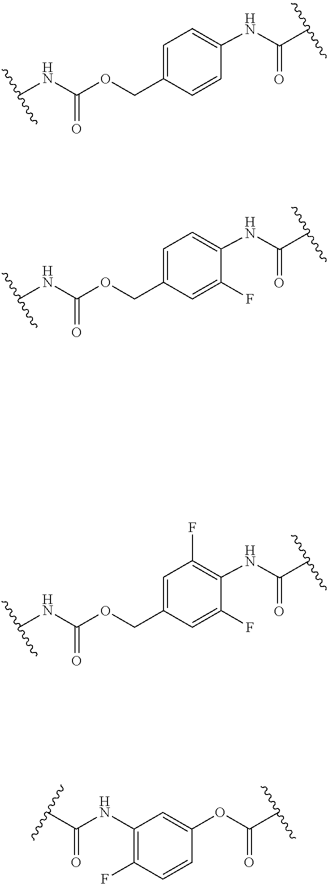 [SMILES: *NC(=O)OCc1ccc(NC(*)=O)cc1 ; *NC(=O)OCc1ccc(NC(*)=O)c(F)c1 ; *NC(=O)OCc1cc(F)c(NC(*)=O)c(F)c1 ; *C(=O)Nc1cc(OC(*)=O)ccc1F] 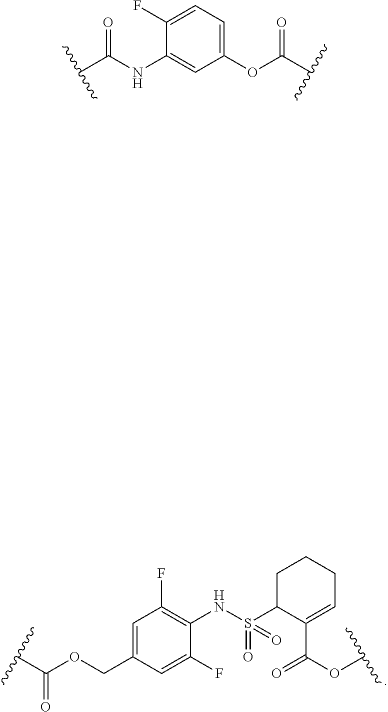 [SMILES: *C(=O)Nc1cc(OC(*)=O)ccc1F ; *OC(=O)C1=CCCCC1S(=O)(=O)Nc1c(F)cc(COC(*)=O)cc1F]
